(19) United States
(12) Patent Application Publication
(10) Pub. No.: US 2012/0126221 A1
(43) Pub. Date: May 24, 2012

(54) CHARGE-TRANSPORTING MATERIAL AND ORGANIC ELECTROLUMINESCENCE DEVICE

(75) Inventors: Tetsu Kitamura, Kanagawa (JP); Toru Watanabe, Kanagawa (JP); Toshihiro Ise, Kanagawa (JP); Hiroo Takizawa, Shizuoka (JP)

(73) Assignee: FUJIFILM CORPORATION, Tokyo (JP)

(21) Appl. No.: 13/388,132

(22) PCT Filed: Jul. 27, 2010
(86) PCT No.: PCT/JP2010/062647

§ 371 Date: Jan. 31, 2012

(30) Foreign Application Priority Data
Jul. 31, 2009 (JP) 2009-180226
Aug. 31, 2009 (JP) 2009-201158
May 7, 2010 (JP) 2010-107586

(51) Int. Cl. H01L 51/54; C07D 403/14; C07D 401/10; C07D 403/10

(52) U.S. Cl. 257/40; 544/242; 544/296; 544/229; 544/180; 546/276.7; 257/E51.026

(57) ABSTRACT

A charge-transporting material contains a compound represented by the following formula (1) in an organic layer, in which the contents of specific halogen-containing compounds are 0.1% or less to the compound represented by formula (1). In formula (1), each of A¹ and A² independently represents N, —CH or —CR; R represents a substituent; L represents a single bond, an arylene group, a cycloalkylene group or an aromatic heterocyclic group; each of R¹ to R⁵ independently represents a substituent; each of n1, n2 and n3 independently represents an integer of 0 to 4; each of n4 and n5 independently represents an integer of 0 to 5; and each of p and q independently represents an integer of 1 to 4.

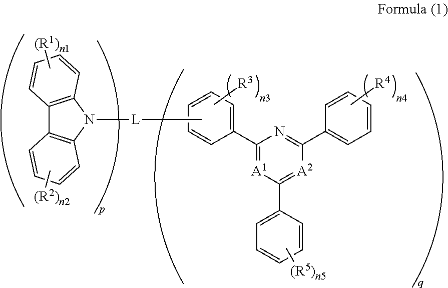

### TECHNICAL FIELD

The present invention relates to a charge-transporting material and an organic electroluminescence device.

### BACKGROUND ART

Since organic electroluminescence devices (hereinafter also referred to as “devices” or “organic EL devices”) are capable of obtaining high luminance emission of light by low voltage driving, they are actively researched and developed. An organic electroluminescence device comprises a pair of electrodes and an organic layer between the pair of electrodes, electrons injected from the cathode and holes injected from the anode are recombined in the organic layer, and generated energy of exciton is used for emission of light.

In recent years, increment in efficiency of devices has been advanced by the use of phosphorescent materials. Doping type devices using a light emitting layer including a host material doped with a light emitting material are also widely adopted.

For example, in patent documents 1 to 3 are proposed organic electroluminescence devices using an iridium complex or a platinum complex as phosphorescent materials and further using a compound having a specific structure containing a nitrogen-containing heterocyclic group and a carbazole structure as a host material to improve light emission efficiency and durability.

Further, an organic electroluminescence device also using a compound having a specific structure containing a nitrogen-containing heterocyclic group and a carbazole structure as an electron-transporting material in an electron-transporting layer to thereby improve light emission efficiency is proposed (refer to patent document 4).

However, organic electroluminescence devices in which light emission efficiency is compatible with durability on a higher level than in the devices described in patent documents 1 to 4 are now required.

Incidentally, patent documents 5 and 6 disclose that durability of an organic electroluminescence device is improved by reducing the concentration of impurities containing halogen-containing compounds in organic compound materials contained in an organic layer. As methods to reduce the concentration of halogen-containing impurities, methods of purifying materials after syntheses of desired organic compound materials (patent documents 5 and 6), and a method of performing reduction treatment of halogen-containing compounds in the materials after syntheses (patent document 6) are proposed.

### RELATED ART DOCUMENT

### Patent Document

Patent Document 1: WO 05/085387
Patent Document 2: WO 03/080760
Patent Document 3: WO 03/078541
Patent Document 4: JP-A-2007-220721
Patent Document 5: Japanese Patent 3290432
Patent Document 6: JP-A-2005-222794

### SUMMARY OF THE INVENTION

### Problems that the Invention is to Solve

In general, one organic compound material contains two or more kinds of halogen-containing impurities but not all of them equally influence the durability of organic electroluminescence device using the organic compound material, and it is not easily known that what a structure of halogen-containing impurity greatly influences the durability of a device.

Further, as described in patent document 6, removal of halogen-containing compound is difficult in many cases and it is necessary to examine proper reducing methods of impurities depending upon organic compound materials.

Regarding the charge-transporting materials having a specific structure containing a nitrogen-containing heterocyclic group and a carbazole structure as described in patent documents 1 to 4, synthesizing methods of the compounds are described in patent documents 1 and 2, which methods comprise coupling a structure containing a nitrogen-containing heterocyclic group substituted with a halogen atom with a carbazole structure containing an aryl group substituted with a boronic acid. However, the purity of these compounds having a specific structure and influences of the contained impurities on a device are not described in patent documents 1 to 4 at all.

On the other hand, as the examples of the substituents capable of substituting for a compound corresponding to the compound represented by formula (1) of the present invention, halogen atoms such as chlorine, bromine and fluorine are enumerated in patent document 2, and it has been known that substitution with a halogen atom in the compound represented by formula (1) does not have so large adverse influence on a device.

An object of the invention is to provide an organic electroluminescence device having excellent light emission efficiency and durability.

Another object of the invention is to provide a charge-transporting material useful for the organic electroluminescence device having excellent light emission efficiency and durability. A further object of the invention is to provide a method for manufacturing a compound useful for the organic electroluminescence device. A still further object of the invention is to provide a light emission apparatus and an illumination apparatus containing the organic electroluminescence device.

### Means for Solving the Problems

In charge-transporting materials including a specific compound containing a nitrogen-containing heterocyclic group and a carbazole structure, it has conventionally been considered that halogen-containing impurities do not exert adverse influences on an organic electroluminescence device. But from the studies, the present inventors have found that impurity compounds having a specific structure among those halogen-containing impurities largely influence the performance of a device and that light emission efficiency and durability of an organic electroluminescence device can be reconciled on a high level by reducing the content of the impurities. The inventors have also found that it is easy to reduce the content of the impurities by obtaining the above specific compound containing a nitrogen-containing heterocyclic group and a carbazole structure according to a specific synthesizing method.

That is, the invention can be attained according to the following means.

[1]

A charge-transporting material comprising a compound represented by the following formula (1), wherein contents of a compound represented by the following formula (I-1) and a compound represented by the following formula (I-2) are respectively 0.1% by mass or less to the compound represented by formula (1):

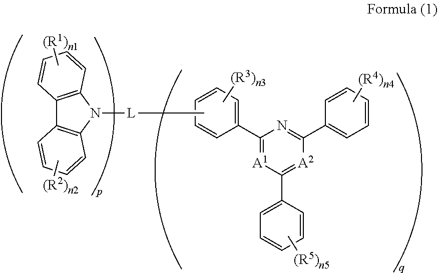

wherein each of A¹ and A² independently represents N, —CH or —CR; R represents a substituent; L represents a single bond, an arylene group, a cycloalkylene group or an aromatic heterocyclic group, a ring may be formed by the carbon atom in the benzene ring to which L is bonded, the atom in L, and further another atom, the other atom is a carbon atom, an oxygen atom or a sulfur atom, and the carbon atom may further have a substituent; each of R¹ to R⁵ independently represents a substituent; each of n1, n2 and n3 independently represents an integer of 0 to 4; each of n4 and n5 independently represents an integer of 0 to 5; and each of p and q independently represents an integer of 1 to 4;

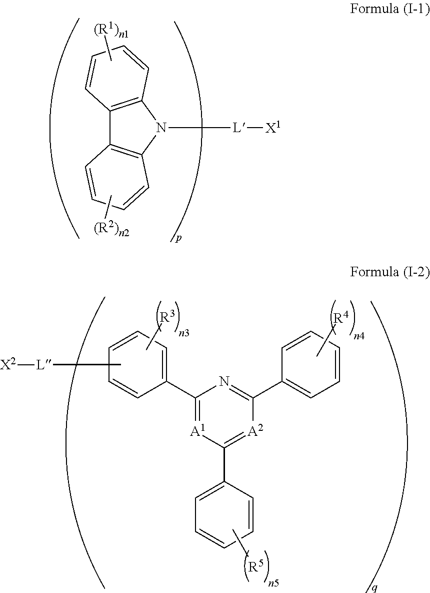

wherein in formulae (I-1) and (1-2) each of A¹, A², R¹ to R⁵, n1 to n5, p and q has the same meaning as in formula (1), and each is the same group or integer with that represented by each of A¹, A², R¹ to R⁵, n1 to n5, p and q in formula (1); each of X¹ and X² independently represents a halogen atom; and each of L′ and L″ has the same meaning with L.

[2]

The charge-transporting material as described in the above [1], wherein the contents of the compound represented by formula (I-1) and the compound represented by formula (I-2) are respectively 0.001% by mass or more and 0.1% by mass or less to the compound represented by formula (1).

[3]

The charge-transporting material as described in the above [1] or [2], wherein in formula (1), either of A¹ and A² represents a nitrogen atom and the other represents —CH or —CR; and R represents a substituent.

[4]

The charge-transporting material described in any of the above [1] to [3], wherein in formula (1), L represents a single bond, a phenylene group, a biphenylene group, or a terphenylene group.

[5]

The charge-transporting material as described in any of the above [1] to [4], wherein in formula (1), each of R¹ to R⁵ independently represents a halogen atom, an alkyl group, an aryl group, an aromatic heterocyclic group, an adamantyl group, a cyano group, a silyl group, or a carbazolyl group.

[6]

The charge-transporting material as described in any of the above [1] to [5], wherein in formula (1), each of R¹ to R⁵ independently represents an alkyl group, an aryl group, a cyano group, or a silyl group.

[7]

The charge-transporting material as described in any of the above [1] to [6], wherein in formula (1), all of n1 to n5 represent 0.

[8]

The charge-transporting material as described in any of the above [1] to [7], wherein the compound represented by formula (1) is a compound represented by the following formula (2):

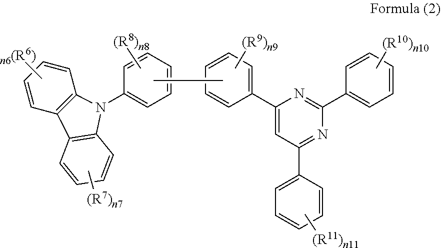

wherein each of R⁶ to R¹¹ independently represents an alkyl group, an aryl group, a cyano group, or a silyl group; each of n6 to n9 independently represents an integer of 0 to 4; and each of n10 and n11 independently represents an integer of 0 to 5.

[9]

The charge-transporting material as described in any of the above [1] to [8], wherein in formula (2), all of n6 to n11 represent 0.

[10]

The charge-transporting material as described in the above [8] or [9], wherein the compound represented by formula (I-1) and the compound represented by formula (I-2) are respectively a compound represented by the following formula (II-1) and a compound represented by the following formula (II-2):

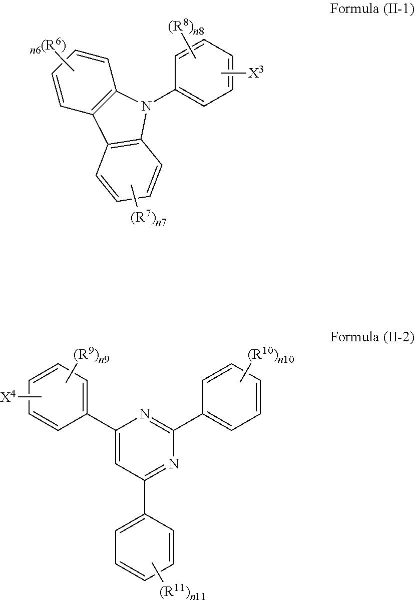

wherein in formulae (II-1) and (II-2), each of X³ and X⁴ independently represents a halogen atom; and each of R⁶ to R¹¹ and n6 to n11 has the same meaning as in formula (2).

[11]

The charge-transporting material as described in any of the above [1] to [10], wherein a molecular weight of the compound represented by formula (1) is 450 or more and 800 or less.

[12]

The charge-transporting material as described in any of the above [1] to [11], wherein a minimum triplet excited state T₁ energy of the compound represented by formula (1) in a film state is 2.69 eV or more and 3.47 eV or less.

[13]

The charge-transporting material as described in any of the above [1] to [12], wherein a glass transition temperature Tg of the compound represented by formula (1) is 80° C. or more and 400° C. or less.

[14]

A method for manufacturing the compound represented by the following formula (2), which comprises a step of performing a coupling reaction of a compound represented by the following formula (M1) with a compound represented by the following formula (M2) by using a palladium catalyst:

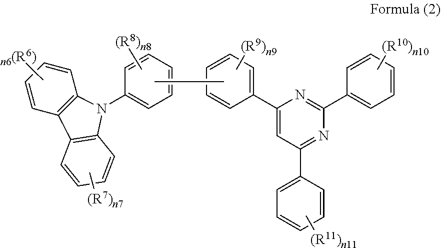

wherein each of R⁶ to R¹¹ independently represents an alkyl group, an aryl group, a cyano group, or a silyl group; each of n6 to n9 independently represents an integer of 0 to 4; and each of n10 and n11 independently represents an integer of 0 to 5;

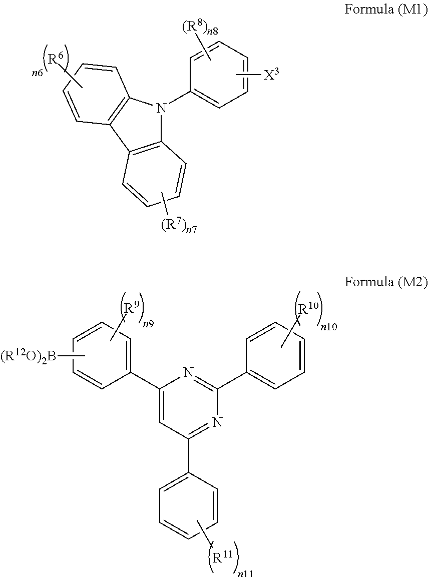

wherein in formulae (M1) and (M2), X³ represents a halogen atom; each of R⁶ to R¹¹ and n6 to n11 has the same meaning as in formula (2); and R¹² represents a hydrogen atom or an alkyl group.

[15]

The method as described in the above [14], which further comprises a step of performing a sublimation purification of a reaction product obtained by the coupling reaction.

[16]

The charge-transporting material as described in the above [8] or [9], wherein the compound represented by formula (2) is obtained by the method as described in the above [14] or [15].

[17]

An organic electroluminescence device comprising:

a pair of electrodes; and

at least one organic layer including a light-emitting layer between the pair of electrodes,

wherein a layer of the at least one organic layers contains the charge-transporting material as described in any of the above [1] to [13] and [16].

[18]

The organic electroluminescence device as described in the above [17], wherein the organic layer contains an electron-transporting layer, and the electron transporting layer contains the charge-transporting material as described in any of the above [1] to [13] and [16].

[19]

The organic electroluminescence device as described in the above [17], wherein the light-emitting layer contains the charge-transporting material as described in any of the above [1] to [13] and [16].

[20]

The organic electroluminescence device as described in any of the above [17] to [19], wherein the light-emitting layer contains a compound represented by the following formula (C-5) as a light-emitting material:

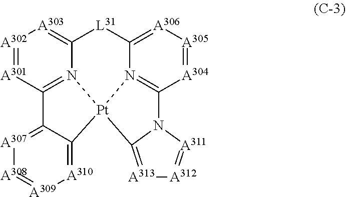

wherein each of A³⁰¹ to A³¹³ independently represents C—R or N; R represents a hydrogen atom or a substituent; and L³¹ represents a single bond or a divalent linking group.

[21]

The organic electroluminescence device as described in the above [20], wherein L³¹ represents a single bond, an alkylene group or an arylene group, the alkylene group or arylene group may further have an alkyl group or an aryl group as the substituent, and when two or more substituents are present, the two or more substituents may be bonded to form a ring.

[22]

The organic electroluminescence device as described in the above [20] or [21], wherein A³⁰² or A³⁰⁵ represents C—R; and R represents a hydrogen atom, an amino group, an alkoxy group, an aryloxy group or a fluorine group.

[23]

The organic electroluminescence device as described in any of the above [20] to [22], wherein A³⁰¹, A³⁰³, A³⁰⁴ or A³⁰⁶ represents C—R; and R represents a hydrogen atom, an amino group, an alkoxy group, an aryloxy group, or a fluorine group.

[24]

The organic electroluminescence device as described in any of the above [20] to [23], wherein when A³⁰⁷, A³⁰⁸, A³⁰⁹ or A³¹⁰ represents C—R, R represents a hydrogen atom, an alkyl group, a perfluoroalkyl group, an aryl group, a dialkylamino group, a cyano group, or a fluorine atom.

[25]

The organic electroluminescence device as described in any of the above [20] to [24], wherein the 6-membered ring formed by A³⁰⁷, A³⁰⁸, A309, A³¹⁰ and two carbon atoms is a benzene ring, a pyridine ring, a pyrazine ring, a pyrimidine ring, or a pyridazine ring.

[26]

The organic electroluminescence device as described in any of the above [20] to [25], wherein when A³¹¹, A³¹² or A³¹³ represents C—R, R represents a hydrogen atom, an alkyl group, a perfluoroalkyl group, an aryl group, a dialkylamino group, a cyano group, or a fluorine atom.

[27]

The organic electroluminescence device as described in any of the above [20] to [26], wherein at least one of A³¹¹, A³¹² and A³¹³ represents N.

[28]

The organic electroluminescence device as described in any of the above [17] to [19], wherein the light-emitting layer contains a compound represented by the following formula (PQ-1) as a light-emitting material:

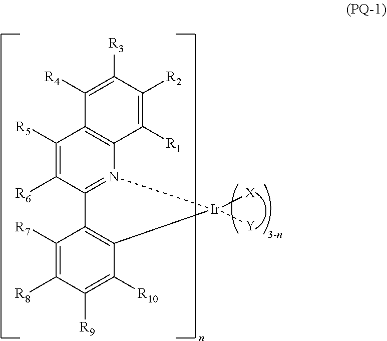

wherein each of R¹ to R¹⁰ independently represents a hydrogen atom or a substituent, and the substituents may be bonded to each other to form a ring; X—Y represents a bidentate monoanionic ligand; and n represents an integer of 1 to 3.

[29]

The organic electroluminescence device as described in the above [28], wherein each of R₁ to R₁₀ independently represents a hydrogen atom, a methyl group, an ethyl group, an isopropyl group, a t-butyl group, a neopentyl group, an isobutyl group, a phenyl group, a naphthyl group, a phenanthryl group, or a tolyl group.

[30]

The organic electroluminescence device as described in the above [28] or [29], wherein X—Y represents acetylacetonate or picolinate.

[31]

The organic electroluminescence device as described in any of the above [28] to [30], wherein the compound represented by formula (PQ-1) is a compound represented by the following formula (PQ-3):

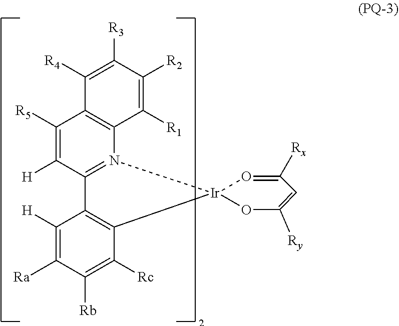

wherein each of R₁ to R₅ has the same meaning as in formula (PQ-1); each of Ra, Rb and Rc independently represents a hydrogen atom or an alkyl group, provided that one of Ra, Rb and Rc represents a hydrogen atom and each of other two represents an alkyl group; and each of Rx and Ry independently represents an alkyl group or a phenyl group.

[32]

A composition comprising the charge-transporting material as described in any of the above [1] to [13] and [16].

[33]

A light emission apparatus comprising the organic electroluminescence device as described in any of the above [17] to [31].

[34]

A display apparatus comprising the organic electroluminescence device as described in any of the above [17] to [31].

[35]

An illumination apparatus comprising the organic electroluminescence device as described in any of the above [17] to [31].

### ADVANTAGE OF THE INVENTION

According to the invention, an organic electroluminescence device having high light emission efficiency and excellent durability can be provided.

### BRIEF DESCRIPTION OF THE DRAWINGS

FIG. 1 is a schematic diagram showing an example of the formation of an organic electroluminescence device according to the invention.

FIG. 2 is a schematic diagram showing an example of an light emission apparatus according to the invention.

FIG. 3 is a schematic diagram showing an example of an illumination apparatus according to the invention.

FIG. 4 is a schematic diagram of a graph showing variation of driving durability to the impurity content (% by mass) of the devices manufactured in the example.

FIG. 5 is a schematic diagram of a graph showing variation of driving durability to the impurity content (% by mass) of the devices manufactured in the example.

FIG. 6 is a schematic diagram of a graph showing variation of driving durability to the impurity content (% by mass) of the devices manufactured in the example.

FIG. 7 is a schematic diagram showing of a graph showing variation of driving durability to the impurity content (% by mass) of the devices manufactured in the example.

FIG. 8 is a schematic diagram showing of a graph showing variation of driving durability to the impurity content (% by mass) of the devices manufactured in the example.

### MODE FOR CARRYING OUT THE INVENTION

An organic electroluminescence device according to the invention is an organic electroluminescence device containing a pair of electrodes and at least one organic layer including a light-emitting layer between the pair of electrodes, and any layer of the organic layers contains the charge-transporting material of the invention. The charge-transporting material is a charge-transporting material containing a compound represented by formula (1), and the contents of a compound represented by formula (I-1) and a compound represented by formula (I-2) are respectively 0.1% by mass or less to the compound represented by formula (1).

### [Charge-Transporting Material]

The charge-transporting material of the invention containing a compound represented by formula (1) will be described below.

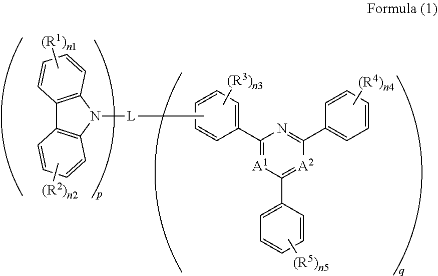

In formula (1), each of A¹ and A² independently represents N, —CH or —CR. R represents a substituent. L represents a single bond, an arylene group, a cycloalkylene group or an aromatic heterocyclic group, and a ring may be formed by the carbon atom in the benzene ring to which L is bonded, the atom in L, and further another atom. The other atom is a carbon atom, an oxygen atom or a sulfur atom, and the carbon atom may further have a substituent. Each of R¹ to R⁵ independently represents a substituent. Each of n1, n2 and n3 independently represents an integer of 0 to 4; and each of n4 and n5 independently represents an integer of 0 to 5. Each of p and q independently represents an integer of 1 to 4.

Formula (1) is described below.

In formula (1), each of A¹ and A² independently represents N, —CH, or R represents a substituent. At least one of A¹ and A² preferably represents a nitrogen atom, more preferably either one of A¹ and A² represents a nitrogen atom and the other represents —CH or —CR, still more preferably A¹ represents —CH or —CR and A² represents a nitrogen atom, and most preferably A¹ represents —CH and A² represents a nitrogen atom.

As specific examples and preferred ranges of the substituents represented by R of —CR, the following substituent group T is exemplified, and most preferred substituents are a t-butyl group, a phenyl group, and a carbazolyl group.

### (Substituent Group T)

Halogen atoms, e.g., fluorine, chlorine, bromine and iodine, a carbazolyl group, a hydroxyl group, an amino group, a nitro group, a cyano group, a silyl group, a carbonyl group, a carboxyl group, an alkyl group, an alkenyl group, an arylalkyl group, an aryl group, an aromatic heterocyclic group, an aralkyl group, an aryloxy group, and an alkyloxy group are exemplified. Each of these substituents may further have any of the above enumerated substituents.

Of these substituents, preferred are a halogen atom, an alkyl group, an aryl group, an aromatic heterocyclic group, an adamantyl group, a cyano group, a silyl group, and a carbazolyl group, e.g., a fluorine atom, a methyl group, a t-butyl group, a phenyl group, a pyridyl group, a pyrazyl group, a pyrimidyl group, an adamantyl group, a cyano group, a trimethylsilyl group, a triphenylsilyl group, a trifluoromethyl group, and a carbazolyl group, more preferred are a fluorine atom, a methyl group, a t-butyl group, a phenyl group, a pyridyl group, a cyano group, a trimethylsilyl group, a triphenylsilyl group, a trifluoromethyl group, and a carbazolyl group, still more preferred are a fluorine atom, a methyl group, a t-butyl group, a phenyl group, a cyano group, a silyl group, a triphenylsilyl group, a trifluoromethyl group, and a carbazolyl group, and still yet preferred are a fluorine atom, a t-butyl group, a phenyl group, a cyano group, a triphenylsilyl group and a carbazolyl group.

L represents a single bond, an arylene group, a cycloalkylene group or an aromatic heterocyclic group, or a combination thereof. Each of these groups may have a substituent, and as the examples of the substituents, the above substituent group T is exemplified. When p+q represents 3 or more in formula (1), L represents a (p+q)-valent group obtained by removing arbitrary (p+q−2) hydrogen atoms from an arylene group, a (p+q)-valent group obtained by removing arbitrary (p+q−2) hydrogen atoms from a cycloalkylene group, or a (p+q)-valent aromatic heterocyclic group.

The arylene group is preferably an arylene group having 6 to 30 carbon atoms and, e.g., a phenylene group, a biphenylene group, a terphenylene group, a naphthylene group, an anthranylene group, a phenanthrylene group, a pyrenylene group, a chrysenylene group, a fluoranthenylene group, and a perfluoroarylene group are exemplified. Of these groups, a phenylene group, a biphenylene group, a terphenylene group, and a perfluoroarylene group are preferred, a phenylene group, a biphenylene group and a terphenylene group are more preferred, and a phenylene group and a biphenylene group are still more preferred.

The cycloalkylene group is preferably a cycloalkylene group having 5 to 30 carbon atoms and, e.g., a cyclopentylene group, a cyclohexylene group and a cycloheptylene group are exemplified, and of these groups, a cyclopentylene group and a cyclohexylene group are preferred, and a cyclohexylene group is more preferred.

The aromatic heterocyclic group is preferably an aromatic heterocyclic group having 2 to 30 carbon atoms, and the examples thereof include a 1-pyrrolyl group, a 2-pyrrolyl group, a 3-pyrrolyl group, a pyrazinyl group, a 2-pyridinyl group, a 3-pyridinyl group, a 4-pyridinyl group, a 1-indolyl group, a 2-indolyl group, a 3-indolyl group, a 4-indolyl group, a 5-indolyl group, a 6-indolyl group, a 7-indolyl group, a 1-isoindolyl group, a 2-isoindolyl group, a 3-isoindolyl group, a 4-isoindolyl group, a 5-isoindolyl group, a 6-isoindolyl group, a 7-isoindolyl group, a 2-furyl group, a 3-furyl group, a 2-benzofuranyl group, a 3-benzofuranyl group, a 4-benzofuranyl group, a 5-benzofuranyl group, a 6-benzofuranyl group, a 7-benzofuranyl group, a 1-isobenzofuranyl group, a 3-isobenzofuranyl group, a 4-isobenzofuranyl group, a 5-isobenzofuranyl group, a 6-isobenzofuranyl group, a 7-isobenzofuranyl group, a 2-quinolyl group, a 3-quinolyl group, a 4-quinolyl group, a 5-quinolyl group, a 6-quinolyl group, a 7-quinolyl group, an 8-quinolyl group, a 1-isoquinolyl group, a 3-isoquinolyl group, a 4-isoquinolyl group, a 5-isoquinolyl group, a 6-isoquinolyl group, a 7-isoquinolyl group, an 8-isoquinolyl group, a 2-quinoxalinyl group, a 5-quinoxalinyl group, a 6-quinoxalinyl group, a 1-carbazolyl group, a 2-carbazolyl group, a 3-carbazolyl group, a 4-carbazolyl group, a 9-carbazolyl group, a 1-phenanthridinyl group, 2-phenanthridinyl group, a 3-phenanthridinyl group, a 4-phenanthridinyl group, a 6-phenanthridinyl group, a 7-phenanthridinyl group, an 8-phenanthridinyl group, a 9-phenanthridinyl group, a 10-phenanthridinyl group, a 1-acridinyl group, a 2-acridinyl group, a 3-acridinyl group, a 4-acridinyl group, a 9-acridinyl group, a 1,7-phenanthrolin-2-yl group, a 1,7-phenanthrolin-3-yl group, a 1,7-phenanthrolin-4-yl group, a 1,7-phenanthrolin-5-yl group, a 1,7-phenanthrolin-6-yl group, a 1,7-phenanthrolin-8-yl group, a 1,7-phenanthrolin-9-yl group, a 1,7-phenanthrolin-10-yl group, a 1,8-phenanthrolin-2-yl group, a 1,8-phenanthrolin-3-yl group, a 1,8-phenanthrolin-4-yl group, a 1,8-phenanthrolin-5-yl group, a 1,8-phenanthrolin-6-yl group, a 1,8-phenanthrolin-7-yl group, a 1,8-phenanthrolin-9-yl group, a 1,8-phenanthrolin-10-yl group, a 1,9-phenanthrolin-2-yl group, a 1,9-phenanthrolin-3-yl group, a 1,9-phenanthrolin-4-yl group, a 1,9-phenanthrolin-5-yl group, a 1,9-phenanthrolin-6-yl group, a 1,9-phenanthrolin-7-yl group, a 1,9-phenanthrolin-8-yl group, a 1,9-phenanthrolin-10-yl group, a 1,10-phenanthrolin-2-yl group, a 1,10-phenanthrolin-3-yl group, a 1,10-phenanthrolin-4-yl group, a 1,10-phenanthrolin-5-yl group, a 2,9-phenanthrolin-1-yl group, a 2,9-phenanthrolin-3-yl group, a 2,9-phenanthrolin-4-yl group, a 2,9-phenanthrolin-5-yl group, a 2,9-phenanthrolin-6-yl group, a 2,9-phenanthrolin-7-yl group, a 2,9-phenanthrolin-8-yl group, a 2,9-phenanthrolin-10-yl group, a 2,8-phenanthrolin-1-yl group, a 2,8-phenanthrolin-3-yl group, a 2,8-phenanthrolin-4-yl group, a 2,8-phenanthrolin-5-yl group, a 2,8-phenanthrolin-6-yl group, a 2,8-phenanthrolin-7-yl group, a 2,8-phenanthrolin-9-yl group, a 2,8-phenanthrolin-10-yl group, a 2,7-phenanthrolin-1-yl group, a 2,7-phenanthrolin-3-yl group, a 2,7-phenanthrolin-4-yl group, a 2,7-phenanthrolin-5-yl group, a 2,7-phenanthrolin-6-yl group, a 2,7-phenanthrolin-8-yl group, a 2,7-phenanthrolin-9-yl group, a 2,7-phenanthrolin-10-yl group, a 1-phenazinyl group, a 2-phenazinyl group, a 1-phenothiazinyl group, a 2-phenothiazinyl group, a 3-phenothiazinyl group, a 4-phenothiazinyl group, a 10-phenothiazinyl group, a 1-phenoxazinyl group, a 2-phenoxazinyl group, a 3-phenoxazinyl group, a 4-phenoxazinyl group, a 10-phenoxazinyl group, a 2-oxazolyl group, a 4-oxazolyl group, a 5-oxazolyl group, a 2-oxadiazolyl group, a 5-oxadiazolyl group, a 3-furazanyl group, a 2-thienyl group, a 3-thienyl group, a 2-methylpyrrol-1-yl group, a 2-methylpyrrol-3-yl group, a 2-methylpyrrol-4-yl group, a 2-methylpyrrol-5-yl group, a 3-methylpyrrol-1-yl group, a 3-methylpyrrol-2-yl group, a 3-methylpyrrol-4-yl group, a 3-methylpyrrol-5-yl group, a 2-t-butylpyrrol-4-yl group, a 3-(2-phenylpropyl)pyrrol-1-yl group, a 2-methyl-1-indolyl group, a 4-methyl-1-indolyl group, a 2-methyl-3-indolyl group, a 4-methyl-3-indolyl group, a 2-t-butyl-1-indolyl group, a 4-t-butyl-1-indolyl group, a 2-t-butyl-3-indolyl group, and a 4-t-butyl-3-indolyl group. Of these groups, a pyridinyl group, a quinolyl group, an indolyl group and a carbazolyl group are preferred, and a pyridinyl group and a carbazolyl group are more preferred.

L preferably represents a single bond or an arylene group, more preferably a single bond, a phenylene group, a biphenylene group or a terphenylene group, still more preferably a single bond, a phenylene group or a biphenylene group, and especially preferably represents a single bond or a phenylene group.

A ring may be formed by the carbon atom in the benzene ring (the benzene ring in which R³ can be substituted) to which L is bonded in formula (1), the atom in L, and further, any other atom. As other atoms to form a ring, a carbon atom, an oxygen atom and a sulfur atom are exemplified. The carbon atom may further be substituted with one or two, preferably two, substituents of substituent group T. As the substituents to be substituted on the carbon atom, an alkyl group, an aryl group, an aromatic heterocyclic group and a cyano group are preferred, an alkyl group and an aryl group are more preferred, a methyl group, an ethyl group, a propyl group, an n-butyl group, a t-butyl group and a phenyl group are still more preferred, a methyl group, a t-butyl group and a phenyl group are yet more preferred, and a methyl group is especially preferred. These substituents may further have the alkyl group or aryl groups described here as the substituent. In the case where the carbon atom is substituted with one substituent, one hydrogen atom is bonded to the carbon atom. When the carbon atom is substituted with two substituents, the two substituents may be the same with or different from each other, but they are preferably the same.

Each of R¹ to R⁵ independently represents a substituent, and the above substituent group T can be exemplified as the examples of the substituents. Each of these groups may further have a substituent, and substituent group T can be exemplified as the examples of the substituents. When each of R¹ to R⁵ is present in the plural, these R¹ to R⁵ may be the same with or different from each other. Further, a ring may be formed in cooperation of R¹ to R⁵.

From the advantage of the invention, each of R¹ to R⁵ preferably represents a halogen atom, an alkyl group, an aryl group, an aromatic heterocyclic group, an adamantyl group, a cyano group, a silyl group, or a carbazolyl group, and more preferably an alkyl group, an aryl group, a cyano group, or a silyl group.

As the specific examples of R¹ to R⁵, a fluorine atom, a methyl group, a t-butyl group, a phenyl group, a pyridyl group, a pyrazyl group, a pyrimidyl group, an adamantyl group, a cyano group, a trimethylsilyl group, a triphenylsilyl group, a trifluoromethyl group, and a carbazolyl group are exemplified. Of these groups, preferred are a fluorine atom, a methyl group, a t-butyl group, a phenyl group, a pyridyl group, a cyano group, a trimethylsilyl group, a triphenylsilyl group, a trifluoromethyl group, and a carbazolyl group, more preferred are a fluorine atom, a methyl group, a t-butyl group, a phenyl group, a cyano group, a silyl group, a triphenylsilyl group, a trifluoromethyl group, and a carbazolyl group, still more preferred are a fluorine atom, a t-butyl group, a phenyl group, a cyano group, a triphenylsilyl group, and a carbazolyl group, yet more preferred are a fluorine atom, a t-butyl group, a phenyl group, a cyano group, and a triphenylsilyl group, and above all preferred are a t-butyl group, a phenyl group, a cyano group, and a triphenylsilyl group.

Each of n1, n2 and n3 independently represents an integer of 0 to 4. Each of n4 and n5 independently represents an integer of 0 to 5. Each of n1 to n5 preferably represents an integer of 0 to 2, more preferably 0 or 1, and still more preferably 0. It is especially preferred that all of n1 to n5 represent 0.

Each of p and q independently represents an integer of 1 to 4. Preferably each of p and q represents 1 to 4, more preferably 1 to 3, and still more preferably 1 or 2.

The compound represented by formula (1) is preferably a compound represented by the following formula (2).

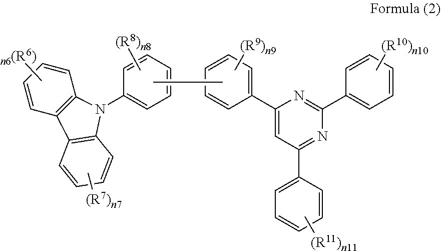

In formula (2), each of R⁶ to R¹¹ independently represents an alkyl group, an aryl group, a cyano group, or a silyl group. Each of n6 to n9 independently represents an integer of 0 to 4, and each of n10 and n11 independently represents an integer of 0 to 5.

Formula (2) is described below.

In formula (2), each of R⁶ to R¹¹ independently represents an alkyl group, an aryl group, a cyano group, or a silyl group. Each of these groups may further have a substituent, and substituent group T can be exemplified as the examples of the substituents.

As the specific examples of R⁶ to R¹¹, a methyl group, a t-butyl group, a phenyl group, a cyano group, a trimethylsilyl group, a triphenylsilyl group, and a trifluoromethyl group are exemplified, and a t-butyl group, a phenyl group, a cyano group, and a triphenylsilyl group are preferred above all.

Each of n6 to n9 independently represents an integer of 0 to 4, and each of n10 and n11 independently represents an integer of 0 to 5. Each of n6 to n11 preferably represents 0 to 2, more preferably 0 or 1, and still more preferably 0. It is especially preferred that all of n6 to n11 represent 0.

From the aspect of driving durability, the compound represented by formula (1) or (2) preferably comprises a carbon atom, a hydrogen atom and a nitrogen atom alone.

The molecular weight of the compound represented by formula (1) is preferably 400 or more and 1,000 or less, more preferably 450 or more and 800 or less, and still more preferably 500 or more and 700 or less. The molecular weight of 400 or more is advantageous to form a high quality amorphous film, and the molecular weight of 1,000 or less is advantageous to improve solubility and sublimation property, and further to improve purity of the compound.

In the case where the compound represented by formula (1) is used as the host material of the light-emitting layer of an organic electroluminescence device or as the charge-transporting material of the layer contiguous to the light-emitting layer, when the energy gap of the compound in a film state (the minimum triplet excited state (T₁) energy in a film state in the case where the light-emitting material is a phosphorescent material) is greater than that of the light-emitting material, quenching of light emission can be prevented, and so advantageous in the improvement of efficiency. On the other hand, from the viewpoint of chemical stability of the compound, energy gap and T₁ energy are preferably not too high. That is, the energy gap of the compound represented by formula (1) in a film state is preferably 2.75 eV (63.5 kcal/mol) or more and 3.90 eV (90 kcal/mol) or less, more preferably 2.82 eV (65 kcal/mol) or more and 3.90 eV (90 kcal/mol) or less, and still more preferably 2.91 eV (67 kcal/mol) or more and 3.90 eV (90 kcal/mol) or less. T₁ energy of the compound represented by formula (1) in a film state is preferably 2.69 eV (62 kcal/mol) or more and 3.47 eV (80 kcal/mol) or less, more preferably 2.75 eV (63.5 kcal/mol) or more and 3.47 eV (80 kcal/mol) or less, and still more preferably 2.82 eV (65 kcal/mol) or more and 3.25 eV (75 kcal/mol) or less. In particular, when a phosphorescent material is used as the light-emitting material, it is preferred that T₁ energy comes into the above range.

T₁ energy can be found from the short wavelength end of the light emission spectrum of phosphorescence of the film of a material. For example, a film is formed in a thickness of about 50 nm by vacuum deposition of a material on a cleaned quartz glass substrate, and the light emission spectrum of phosphorescence of the film is measured with F-7000 Hitachi fluorescence spectrophotometer (manufactured by Hitachi High Technologies Corporation) under a liquid nitrogen temperature. T₁ energy can be found by converting the rising wavelength on the short wavelength side of the obtained light emission spectrum into an energy unit.

From the aspect of driving an organic electroluminescence device stably at high temperature driving time or to the calorification during driving of the device, the glass transition temperature (Tg) of the compound represented by formula (1) is preferably 80° C. or more and 400° C. or less, more preferably 100° C. or more and 400° C. or less, and still more preferably 120° C. or more and 400° C. or less.

The specific examples of the compound represented by formula (1) are shown below, but the invention is not restricted thereto.

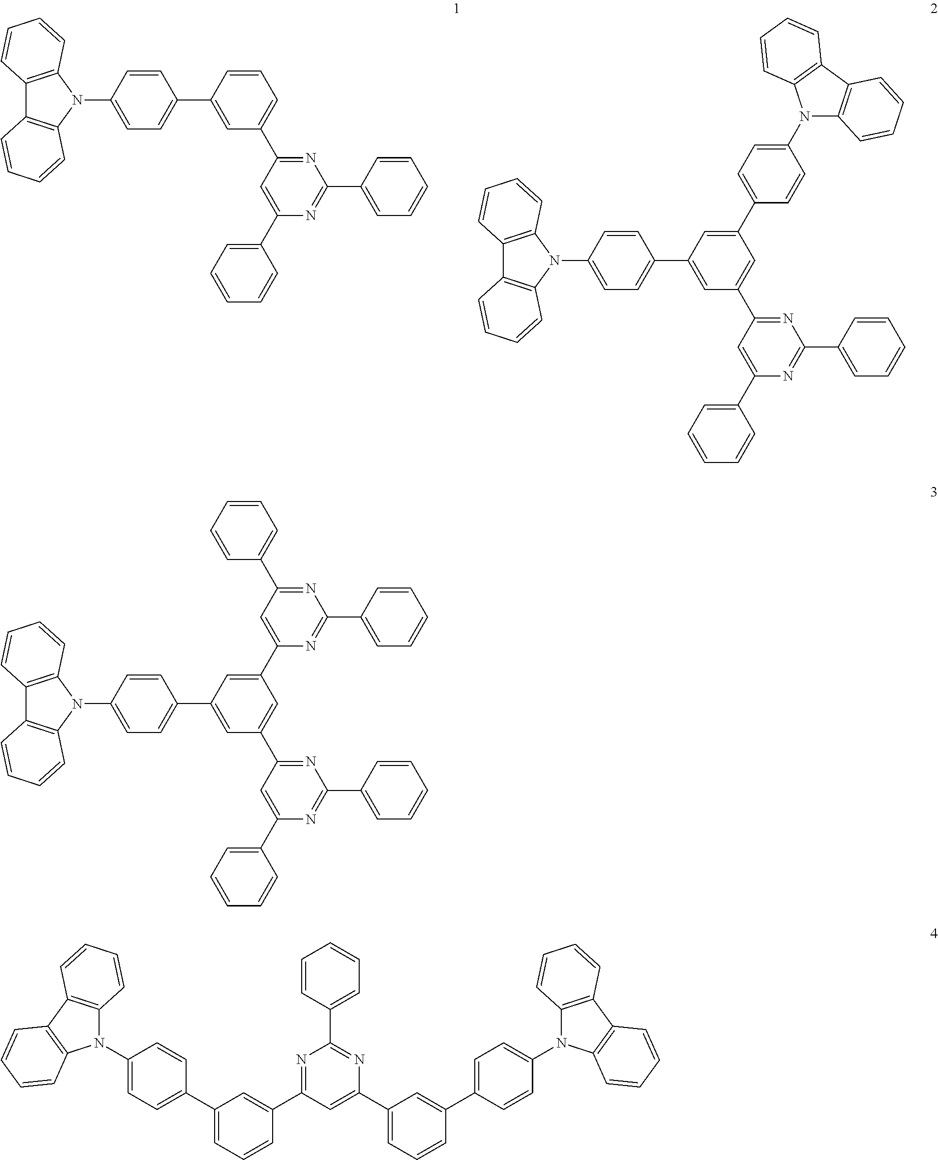

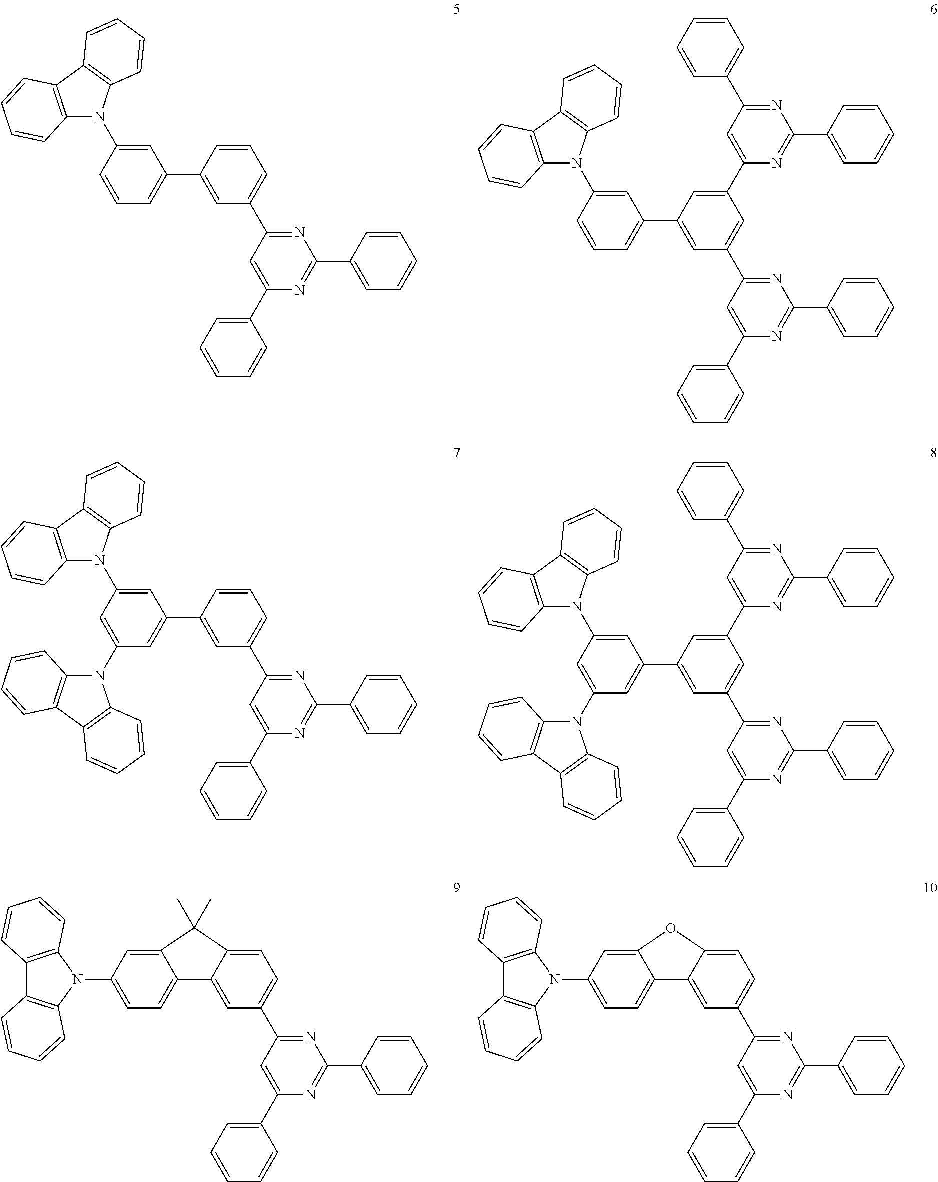

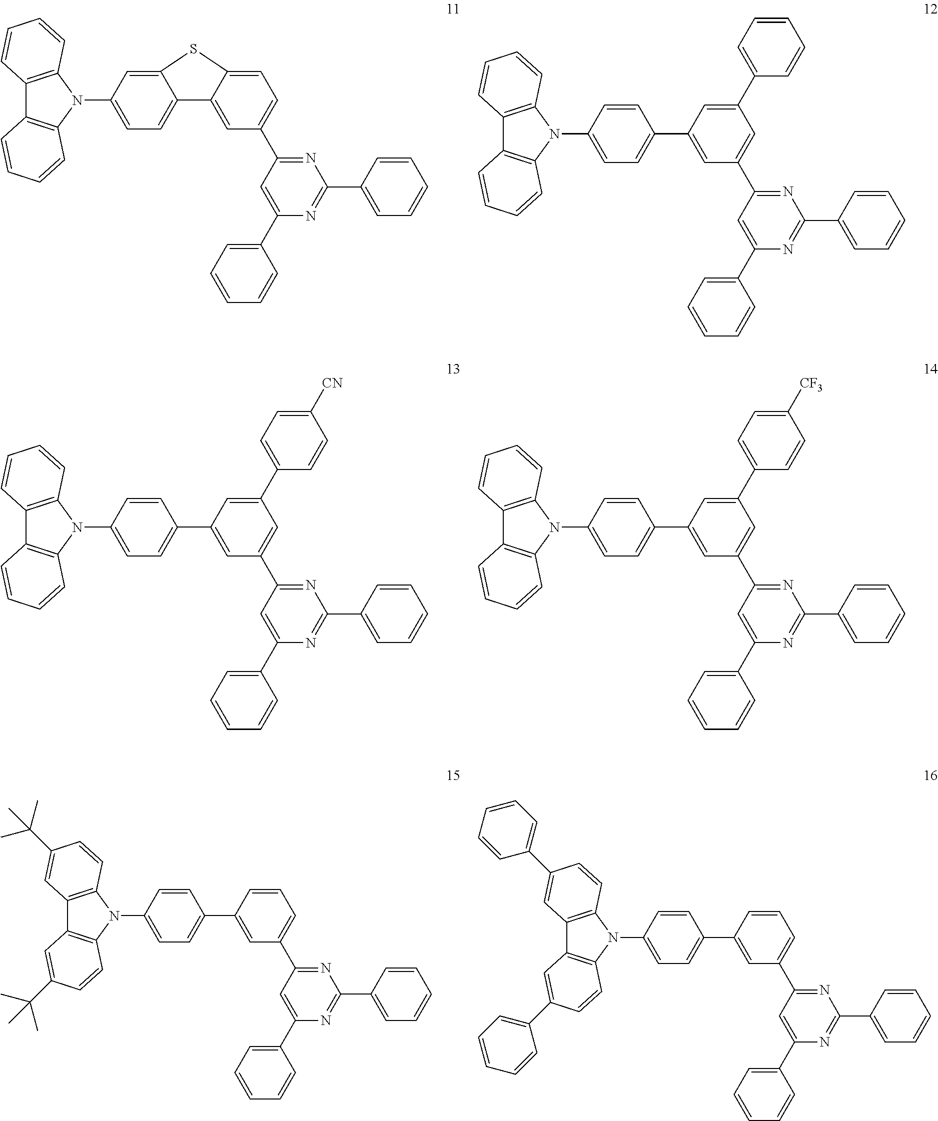

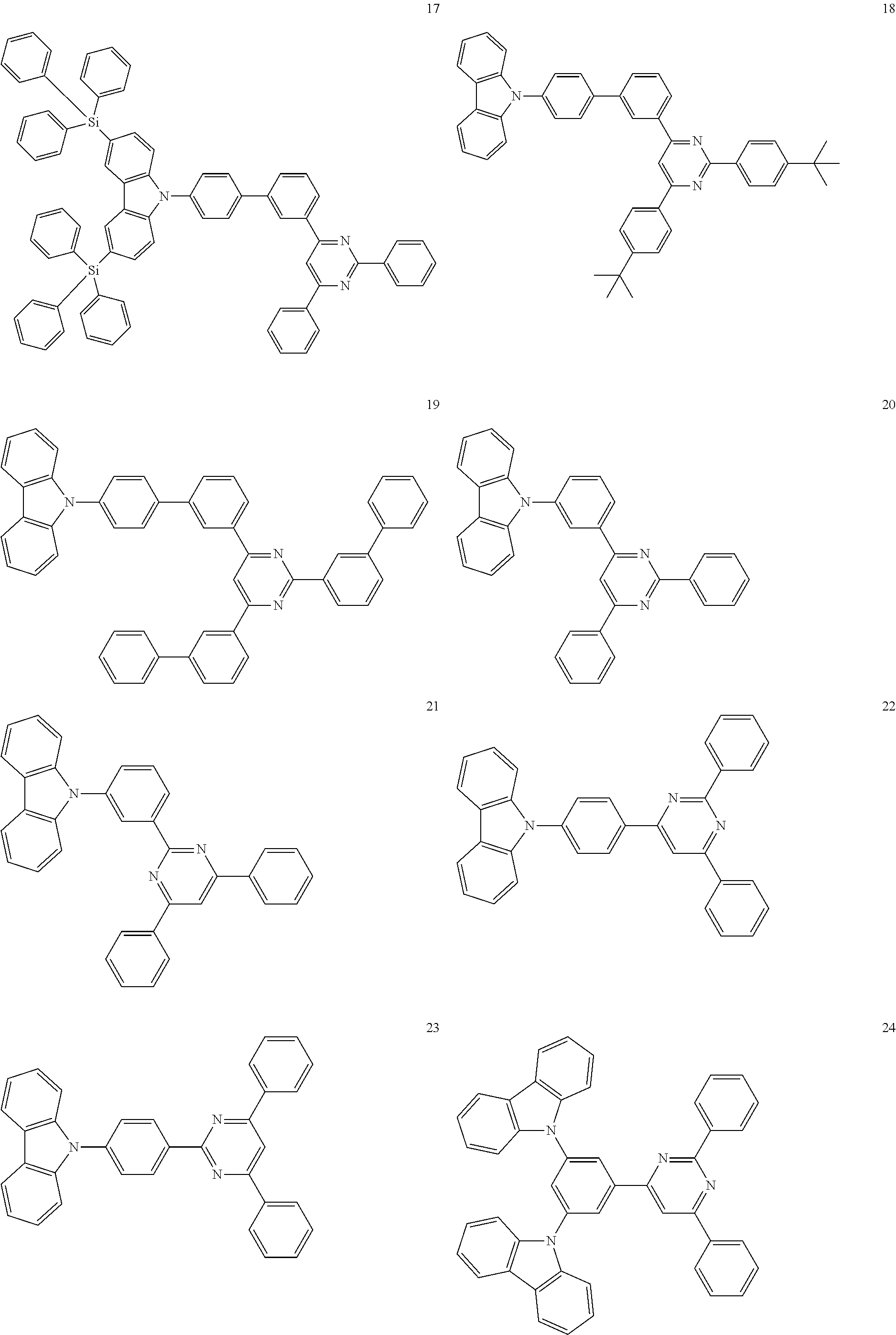

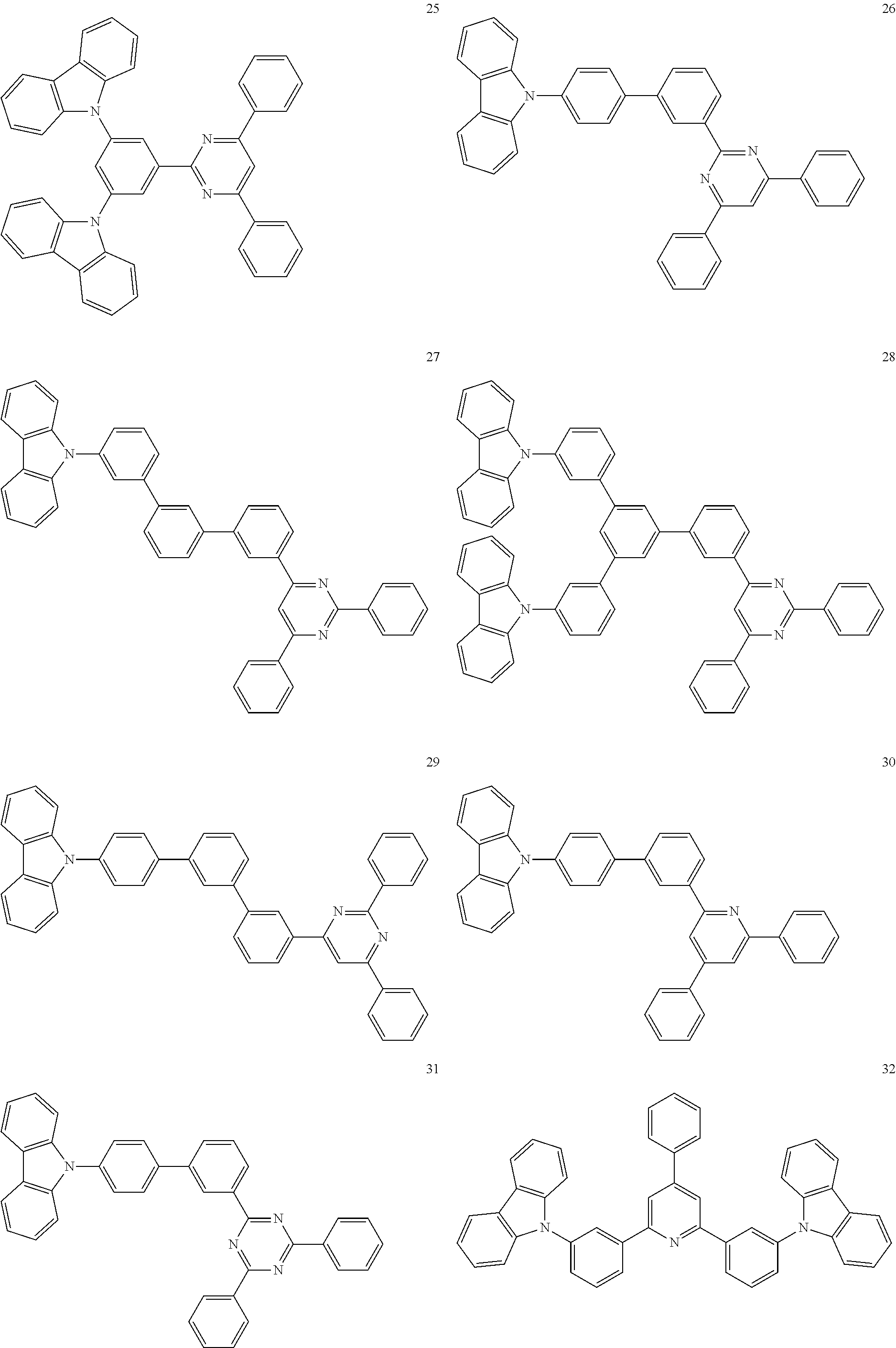

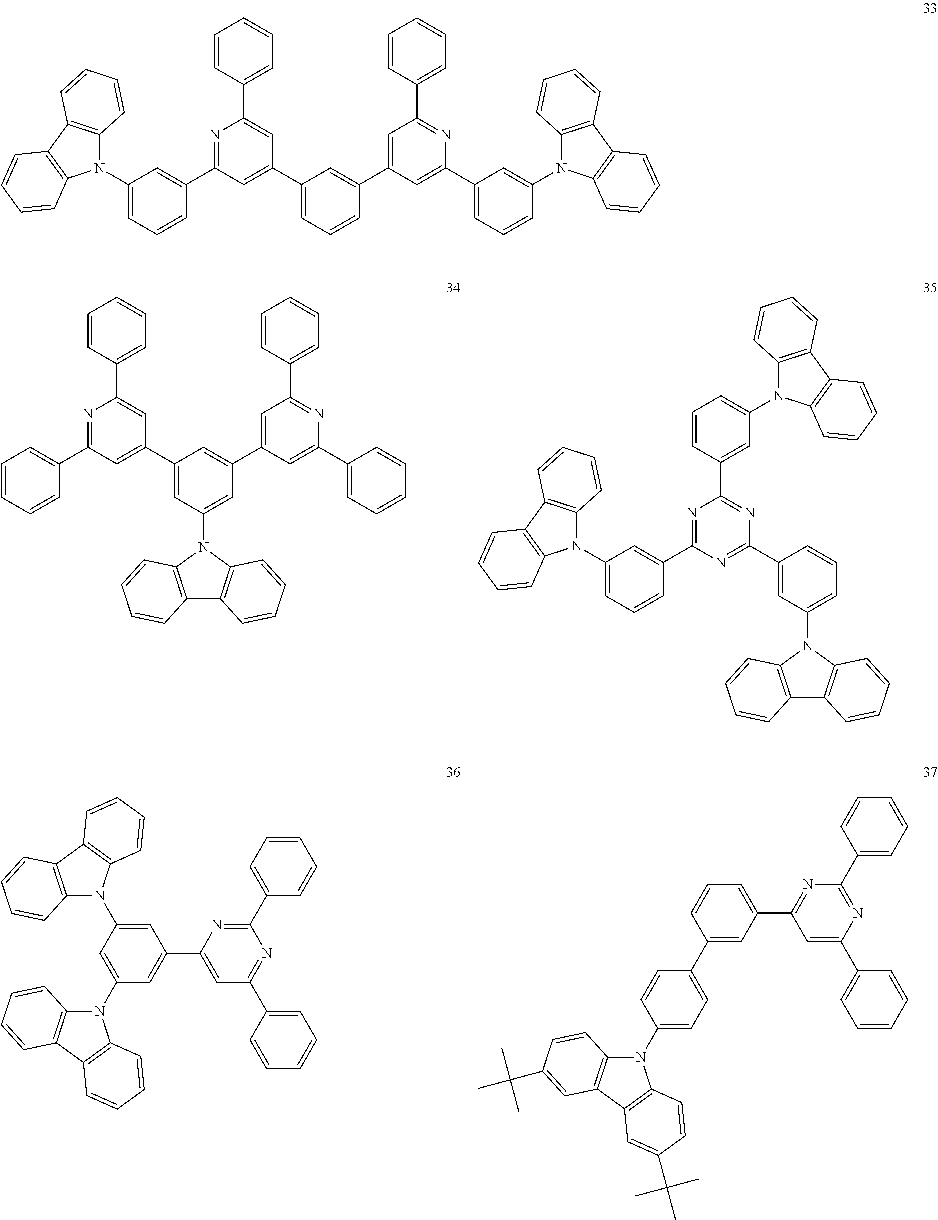

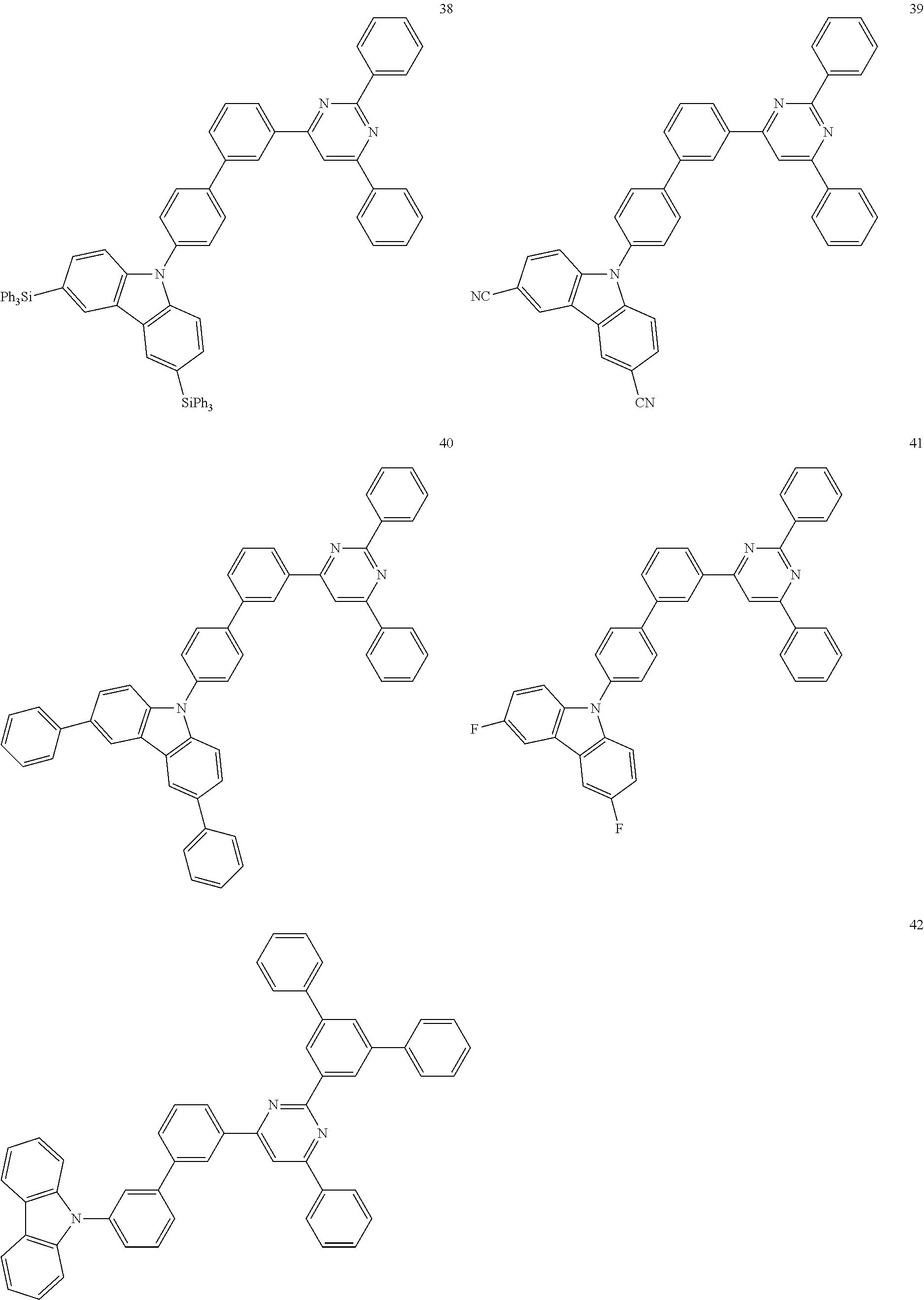

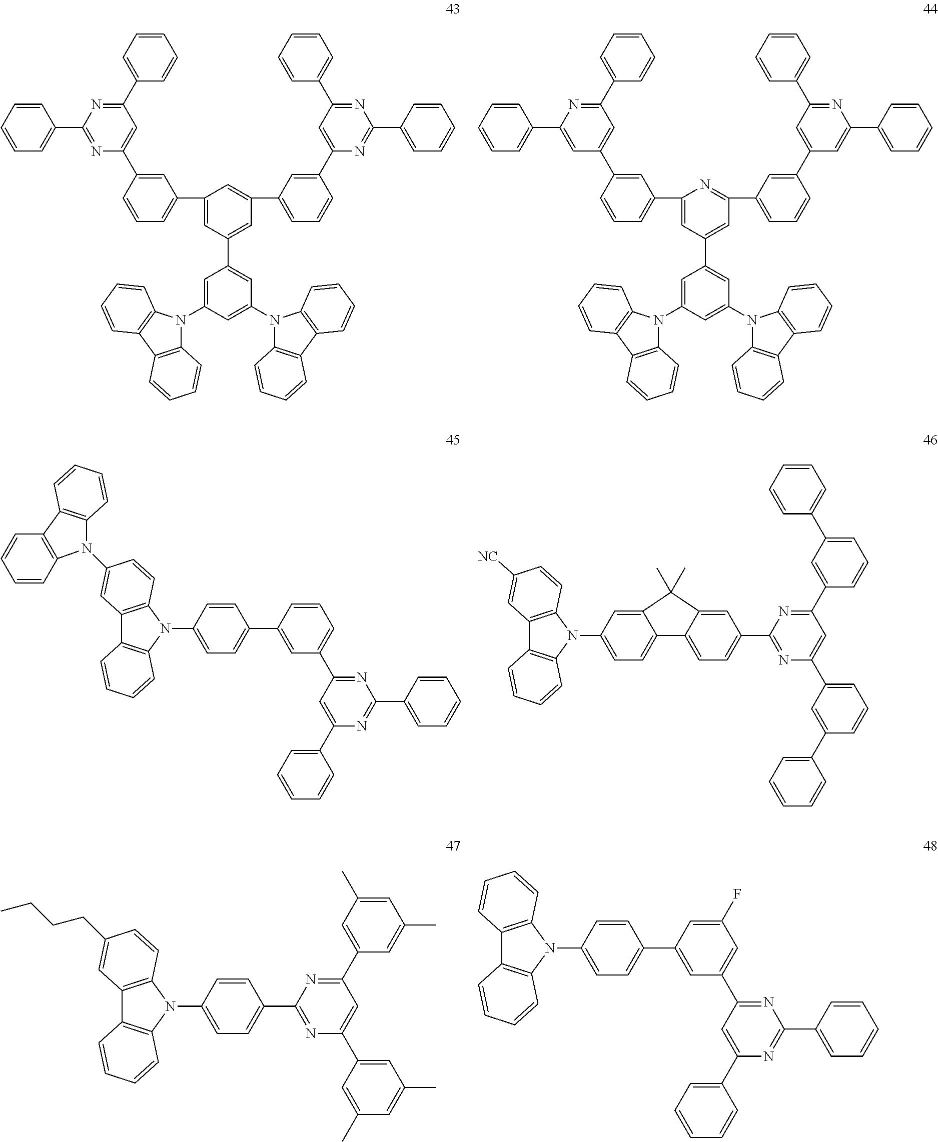

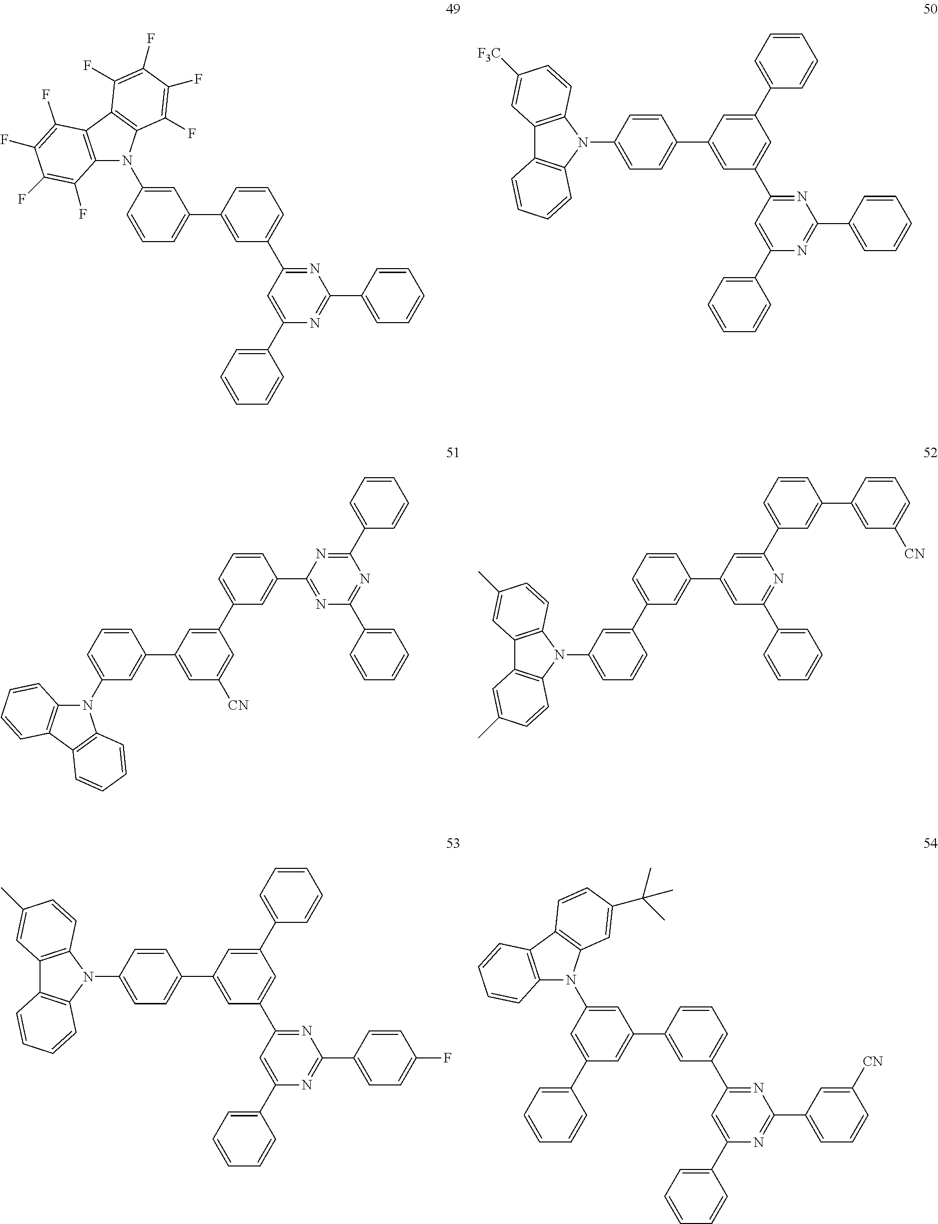

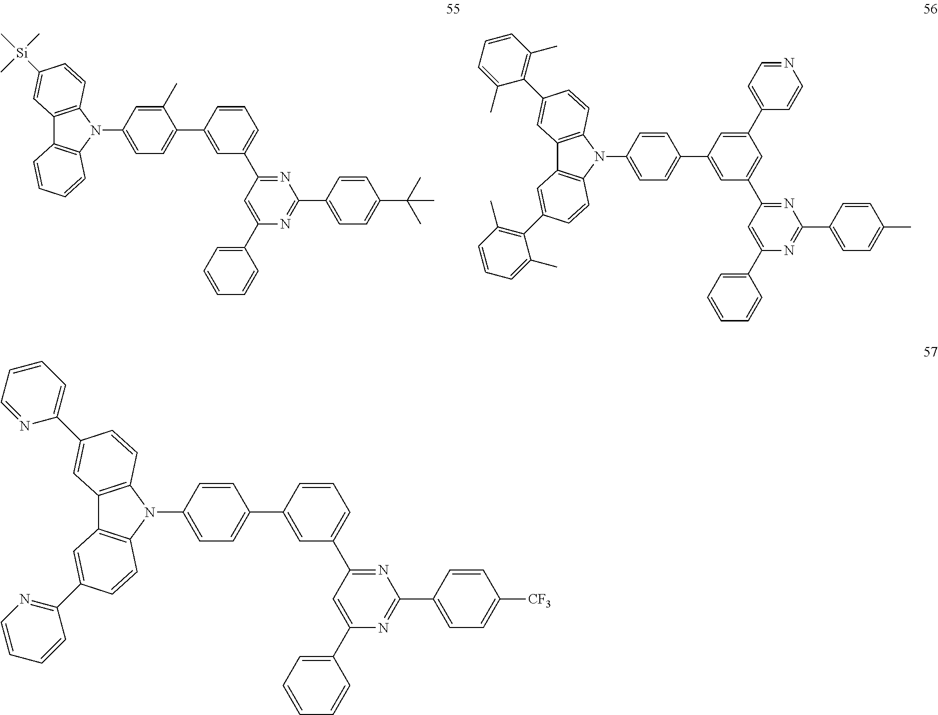

In the next place, the impurities in the charge-transporting material containing the compound represented by formula (1) will be described.

In the invention, the contents of a compound represented by the following formula (I-1) and a compound represented by the following formula (I-2) are respectively 0.1% by mass or less to the compound represented by formula (1) in a charge-transporting material containing the compound represented by formula (1).

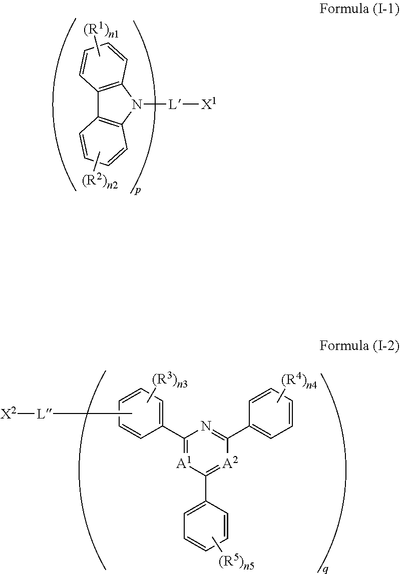

In formulae (I-1) and (1-2), each of A¹, A², R¹ to R⁵, n1 to n5, p and q has the same meaning as in formula (1), and each is the same group or integer with that represented by each of A¹, A², R¹ to R⁵, n1 to n5, p and q in formula (1). Each of X¹ and X² independently represents a halogen atom. Each of L′ and L″ has the same meaning with L.

The compound represented by formula (1) can be synthesized by coupling aryl halide with aryl boronic acid (or boronic acid ester) or carbazole as described in WO 05/085387 and WO 03/080760. At this time, aryl halide which is an intermediate in synthesis (for example, aryl halide having a carbazole part or aryl halide having a pyrimidine part) may be formed as an impurity. According to the examinations by the present inventors, it has been found that when such aryl halide is present in a charge-transporting material containing the compound represented by formula (1) in an amount exceeding 0.1% by mass, the characteristics of the device such as light emission efficiency and durability of the organic electroluminescence device are affected for the reason that the excessive amount of halogen atoms causes charge trapping and reactivity becomes high and, in particular, durability is deteriorated and it becomes difficult to reconcile light emission efficiency with durability on a high level. Further when this aryl halide is a compound represented by formula (I-1) and/or a compound represented by formula (I-2), influence on the characteristics of the device is conspicuously large. Accordingly, it is necessary that the contents of these compounds are respectively 0.1% by mass or less to the compound represented by formula (1). Preferably the contents of these compounds are respectively 0.05% by mass or less, and more preferably 0.03% by mass or less.

Formula (I-1) and (1-2) will be described below.

In the formulae, each of R¹ to R⁵ and n1 to n5 has the same meaning as in formula (1), and each is the same group or integer with that represented by each of A¹, A², R¹ to R⁵, n1 to n5, p and q in formula (1).

Each of X¹ and X² independently represents a halogen atom (a fluorine atom, a chlorine atom, a bromine atom, an iodine atom). When the halogen atoms are a chlorine atom, a bromine atom, and an iodine atom, the characteristics of the device are more largely affected, and a bromine atom and an iodine atom exert still further serious influence upon the characteristics. Accordingly, effect of making the contents of the compound represented by formula (I-1) and the compound represented by formula (I-2) respectively 0.1% by mass or less to the compound represented by formula (1) is greater.

L′ and L″ has the same meaning with L in formula (1). The compound represented by formula (I-1) and/or the compound represented by formula (I-2) is the starting material and intermediate synthetic product at the time of the synthesis of the compound represented by formula (1). In this case, each of L′ and L″ represents a single bond or a divalent linking group having the partial structure of L in formula (1). For example, when L represents biphenylene, each of L′ and L″ represents any of a single bond, phenylene and biphenylene.

When the compound represented by formula (I-1) and the compound represented by formula (I-2) are respectively a compound represented by the following formula (II-1) and a compound represented by formula (II-2), it is preferred from the viewpoint of durability of a device to make the contents of these compounds 0.1% by mass or less to the compound represented by formula (1) or (2). More preferably the contents of these compounds are respectively 0.05% by mass or less to the compound represented by formula (1) or (2), and still more preferably 0.03% by mass or less.

The compound represented by formula (II-1) and the compound represented by formula (II-2) are aryl halides largely affecting the characteristics of device as impurities when the compound represented by formula (1) is the compound represented by formula (2).

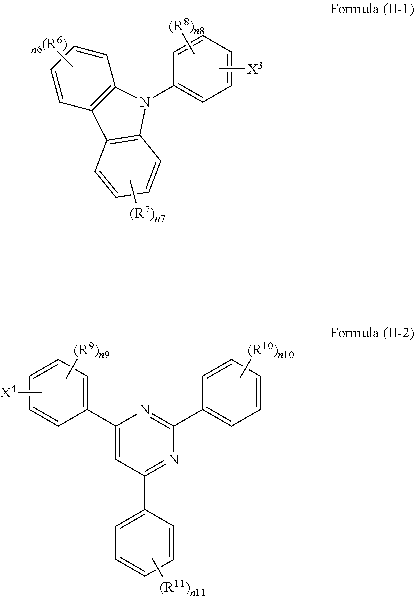

In formulae (II-1) and (II-2), each of X³ and X⁴ independently represents a halogen atom. Each of R⁶ to R¹¹ and n6 to n11 has the same meaning as in formula (2).

Formulae (II-1) and (II-2) are described below.

In the formulae, each of R⁶ to R¹¹ and n6 to n11 has the same meaning as in formula (2). When each of R⁶ to R¹¹ represents an alkyl group, an aryl group, a cyano group, or a silyl group and/or each of n6 to n11 represents an integer of 0 to 3, the effect of making the contents of the compound represented by formula (II-1) and the compound represented by formula (II-2) respectively 0.1% by mass or less to the compound represented by formula (1) or (2) is greater. When each of R⁶ to R¹¹ represents an alkyl group or an aryl group and/or each of n6 to n11 represents 0 or 1, the effect is further greater.

Each of X³ and X⁴ independently represents a halogen atom (a fluorine atom, a chlorine atom, a bromine atom, an iodine atom). When the halogen atoms are a chlorine atom, a bromine atom, and an iodine atom, the characteristics of the device are more largely affected, and a bromine atom and an iodine atom exert still further serious influence upon the characteristics. However, even in such a case in the invention, by making the contents of the compounds represented by formula (II-1) and formula (II-2) respectively 0.1% by mass or less to the compound represented by formula (1) or (2), the influence on the characteristics of the device can be controlled and durability can be improved.

The contents of aryl halides and other impurities of the compounds represented by any of formulae (I-1), (I-2), (II-1) and (II-2) in the charge-transporting material of the invention and the purity of the charge-transporting material of the invention can be found by high performance liquid chromatography (HPLC). In the invention, the area ratio of absorption strength at 254 nm is used as the index of impurity content and purity. The peak position of aryl halide can be confirmed by comparison with the aryl halide which is an intermediate in synthesis of the compound represented by formula (1) or (2) of the charge-transporting material of the invention. The structures of peaks of other impurities can be estimated by liquid chromatography/mass spectrometry (LC/MS).

As aryl halides contained in the charge-transporting material of the invention as impurities, starting materials for synthesizing the compounds of formulae (I-1), (I-2), (II-1) and (II-2) and aryl halides used in intermediates can be enumerated other than the compounds represented by formulae (I-1), (I-2), (II-1) and (II-2). Specifically iodobromobenzene and p-bromobenzaldehyde can be exemplified.

When aryl halides other than those of the compounds represented by formulae (I-1), (I-2), (II-1) and (II-2) are contained as impurities in the charge-transporting material of the invention, the content of all aryl halides is preferably 0.2% by mass or less to the compound represented by formula (1) or (2), more preferably 0.1% by mass or less, and still more preferably 0.05% by mass or less. When the content exceeds 0.2% by mass, there are cases where the characteristics of the device such as efficiency and durability are adversely affected for the reason that the excessive amount causes charge trapping and reactivity becomes high.

Influence on the characteristics of the device due to impurities besides these aryl halides is small even if they are contained. As other impurities, compounds obtained by substituting the halogen atoms in the compounds represented by formulae (I-1), (I-2), (II-1) and (II-2) with hydrogen atoms are exemplified. The content of impurities other than the aryl halides in the charge-transporting material of the invention is preferably 0.5% by mass or less, more preferably 0.3% by mass or less, and still more preferably 0.2% by mass or less.

Preferably, the gross content of impurities at all (aryl halides and impurities other than aryl halides) contained in the charge-transporting material of the invention is preferably 1.0% by mass or less to the compound represented by formula (1) or (2), more preferably 0.5% by mass or less, and still more preferably 0.1% by mass or less.

The content of the impurities in the charge-transporting material of the invention is ideally 0% by mass. On the other hand, it is also impossible to really measure 0% by mass of impurity. Further, from the aspect of environmental load affected by the increase in the number of manufacturing process and purification process and increase in energy to be used, a trace amount of impurities is preferably rather present in the charge-transporting material of the invention depending upon the kinds of impurities. Compounds not containing halogen atoms are exemplified as such impurities. The content is preferably 0.01% by mass or more and 0.2% by mass or less respectively to the compound represented by formula (1) or (2), more preferably 0.01% by mass or more and 0.1% by mass or less, and still more preferably 0.01% by mass or more and 0.05% by mass or less.

From the aspect of environmental load affected by the increase in the number of manufacturing process and purification process and increase in energy to be used, the compound represented by any of formulae (I-1), (1-2), (II-1) and (II-2) of the invention is also preferably contained in the charge-transporting material of the invention in an extremely small amount. Accordingly, from both points of the improvement of durability and environmental load control, the content of each compound represented by any of formulae (I-1), (I-2), (II-1) and (II-2) of the invention is preferably 0.001% by mass or more and 0.1% by mass or less respectively to the compound represented by formula (1) or (2), more preferably 0.001% by mass or more and 0.05% by mass or less, and still more preferably 0.001% by mass or more and 0.03% by mass or less.

The purity of the charge-transporting material of the invention is preferably 99.0% by mass or more, more preferably 99.5% by mass or more, and still more preferably 99.9% by mass or more.

The compound represented by formula (1) of the invention can be synthesized according to various methods, e.g., the methods described in WO 05/085387 and WO 03/080760.

The synthesized compound is preferably purified by sublimation purification after having been subjected to purification treatment by column chromatography, recrystallization and the like. Not only organic impurities can be separated but also inorganic salts and residual solvents can be effectually removed by sublimation purification.

### [Manufacturing Method of Compound Represented by Formula (2)]

The compound represented by formula (2) of the invention can be synthesized by coupling aryl halide having a pyrimidine part with aryl boronic acid having a carbazole part as described in WO 05/085387 and WO 03/080760.

For example, exemplified compound 1 for use in the examples later can be synthesized with m-bromobenzaldehyde as the starting material according to the method described in WO 05/085387, paragraphs [0074] to [0075] (page 45, line 11 to page 46, line 18). Further, exemplified compound 2 can be synthesized with m-bromobenzaldehyde as the starting material according to the method described in WO 05/085387, paragraphs [0078] to [0079] (page 47, line 11 to page 46, line 23).

In the manufacturing method of the invention, aryl halide having a carbazole part and aryl boronic acid (or boronic acid ester) having a pyrimidine part are subjected to coupling reaction. That is to say, a compound represented by the following formula (M1) and a compound represented by the following formula (M2) are subjected to coupling reaction with a palladium catalyst.

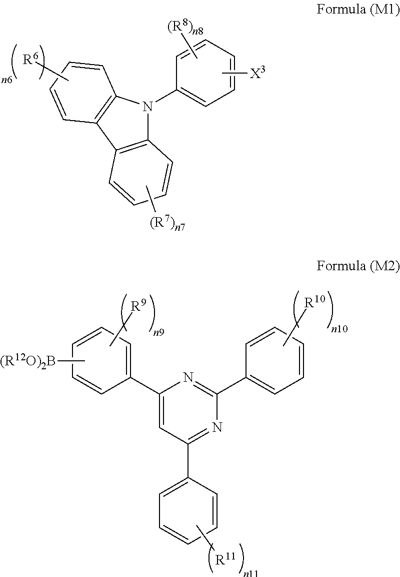

In formulae (M1) and (M2), X³ represents a halogen atom. Each of R⁶ to R¹¹ and n6 to n11 has the same meaning as in formula (2). R¹² represents a hydrogen atom or an alkyl group.

Formulae (M1) and (M2) will be described below.

Each of R⁶ to R¹¹ and n6 to n11 has the same meaning as in formula (2).

R¹² represents a hydrogen atom or an alkyl group. A ring may be formed in cooperation of two R¹². As the alkyl group represented by R¹², a methyl group, an ethyl group, a propyl group, a butyl group, a cyclohexyl group, and a group forming a pinacol ring by two R¹² by linking to each other can be exemplified. R¹² preferably represents a hydrogen atom, a methyl group, an ethyl group, or a group forming a pinacol ring by two R¹² by linking to each other, and more preferably represents a hydrogen atom, a methyl group, or a group forming a pinacol ring by two R¹² by linking to each other.

X³ represents a halogen atom (a fluorine atom, a chlorine atom, a bromine atom, or an iodine atom), preferably a chlorine atom, a bromine atom or an iodine atom, and more preferably a bromine atom.

The reaction conditions of the above coupling reaction described in Chem. Rev., 1995, 95, 2457-2483 can be used. Preferred reaction conditions are described below.

As the palladium catalyst, divalent palladium salts or 0-valent palladium salts are used. As divalent palladium, palladium acetate and dichlorobistriphenyl-phosphine palladium, and as 0-valent palladium, tetrakistriphenylphosphine palladium and bis(dibenzylidene acetone) palladium are exemplified. Of these palladium catalysts, palladium acetate and tetrakis(triphenylphosphine) palladium are preferred.

Reaction solvents are not especially restricted, but water; aromatic hydrocarbons, e.g., benzene, toluene, and xylene; halogenated hydrocarbons, e.g., dichloroethane and chloroform; ethers, e.g., tetrahydrofuran, 1,2-dimethoxyethane, 1,4-dioxane, and diethyl ether; alcohols, e.g., methanol, ethanol, and isopropyl alcohol; and esters, e.g., ethyl acetate and butyl acetate are exemplified. Of these solvents, water, aromatic hydrocarbons and ethers are preferred. These solvents may be used as mixtures of two or more kinds.

Reaction temperature is not especially restricted and generally reaction is carried out between 0° C. and the boiling temperature of the solvent. In the case where decomposition of the produced product does not occur, the reaction is preferably performed at the temperature near the boiling temperature of the solvent for increasing reaction speed.

In the above reaction, if necessary, ligands may further be added. As the ligands, a phosphine ligand and a carbene ligand are exemplified, and a phosphine ligand is preferred of them.

The use amount of these ligands is generally 0.5 molar times to 20 molar times to the palladium catalyst to be used, preferably 1 molar time to 10 molar times, and more preferably 1 molar time to 5 molar times.

Bases for use in the above reaction are not especially restricted. Specifically, alkali metal hydroxides, e.g., lithium hydroxide, sodium hydroxide and potassium hydroxide, alkaline earth metal hydroxides, e.g., calcium hydroxide and barium hydroxide, alkali metal hydrogencarbonates, e.g., sodium hydrogencarbonate and potassium hydrogencarbonate, alkaline earth metal hydrogencarbonates, e.g., calcium hydrogencarbonate and barium hydrogencarbonate, alkali metal carbonates, e.g., sodium carbonate and calcium carbonate, alkaline earth metal carbonates, e.g., calcium carbonate and barium carbonate, and phosphates, e.g., sodium phosphate and potassium phosphate are exemplified. Alkali metal hydrogencarbonates, alkali metal carbonates and phosphates are preferred of these bases.

The use amount of bases is generally 0.1 molar times to 50 molar times to compound (M1), preferably 1 molar time to 20 molar times, and more preferably 2 molar times to 10 molar times.

The compound represented by formula (2) is synthesized by mixing the compound represented by formula (M1), the compound represented by formula (M2), the above palladium catalyst and the solvent, and reacting at the above temperature.

After the coupling reaction in the invention, the product formed by the reaction is preferably subjected to sublimation purification. A charge-transporting material containing the compounds represented by formulae (I-1) and (1-2), which adversely affect the characteristics of the device, of the content of respectively 0.1% by mass or less to the compound of formula (1) can be preferably obtained by performing sublimation purification after column chromatography and recrystallization.

In the manufacturing method of the invention, the compound of (M1) contains halogen atoms but, from the examinations by the inventors, impurities ascribing to the aryl halide of the carbazole part can be easily removed, and so the sublimation purification is advantageous for the adjustment of the contents of impurities.

In the sublimation purification, by forming temperature gradient in the system taking the position where the sample of purification object is settled as basis, a high purity product can be obtained in the region (fraction) far from the settled position. At that time, it is preferred to introduce gas such as Ar or nitrogen into the system. The pressure in the system is preferably 1 Pa to 10⁻⁵ Pa, and more preferably 1 Pa to 10⁻³ Pa.

### [Uses for the Charge-Transporting Material of the Invention]

The charge-transporting materials according to the invention can be preferably used in electrophotography, organic transistors, organic photoelectric conversion devices (energy conversion uses and sensor uses), and organic electronic devices such as organic electroluminescence devices, and it is especially preferred to use them in organic electroluminescence devices.

In an organic electroluminescence device, the charge-transporting material of the invention may be contained in any layer of organic layers. Preferred is a case where the material is used in any of a hole injecting layer, a hole transporting layer, a light-emitting layer, an electron transporting layer and an electron injecting layer, more preferred is a case of using the material in any of a light-emitting layer, an electron transporting layer and an electron injecting layer, and still more preferred is to use the material in a light-emitting layer or an electron transporting layer.

When the compound represented by formula (1) is contained in a light-emitting layer, the compound represented by formula (1) of the invention is preferably contained in an amount of 10% by mass to 99% by mass to total mass of the light-emitting layer, more preferably 40% by mass to 95% by mass, and still more preferably 70% by mass to 90% by mass.

Further, when the compound represented by formula (1) is contained in a layer other than a light-emitting layer, the content is preferably 60% by mass to 100% by mass to total mass of the layer, more preferably 70% by mass to 100% by mass, and still more preferably 85% by mass to 100% by mass.

### [Composition Containing the Charge-Transporting Material of the Invention]

The invention also relates to a composition containing the charge-transporting material. The content of the compound represented by formula (1) in the composition of the invention is preferably 30% by mass to 99% by mass, more preferably 50% by mass to 95% by mass, and still more preferably 70% by mass to 90% by mass. Other components which may be contained in the composition of the invention may be organic or inorganic. As organic materials, host materials described later, fluorescent materials, phosphorescent materials, and materials exemplified as hydrocarbon materials can be applied, and host materials and hydrocarbon materials are preferred.

The composition of the invention can be formed to the organic layer of an organic electroluminescence device according to dry film-forming methods such as a vacuum deposition method, a sputtering method, etc., and a transfer method, a printing method, etc.

### [Organic Electroluminescence Device]

The organic electroluminescence device according to the invention will be described in detail below.

The organic electroluminescence device according to the invention contains a pair of electrodes and organic layers including a light-emitting layer between the pair of electrodes. From the property of a luminescence device, at least one electrode of the pair of electrodes of the anode and cathode is preferably transparent or translucent.

As organic layers, a hole-injecting layer, a hole-transporting layer, a blocking layer (a hole-blocking layer, an exciton-blocking layer), and an electron-transporting layer are exemplified besides a light-emitting layer. These organic layers may be provided as two or more layers, and when two or more layers are formed, they may be formed of the same material or each layer may be formed with a different material.

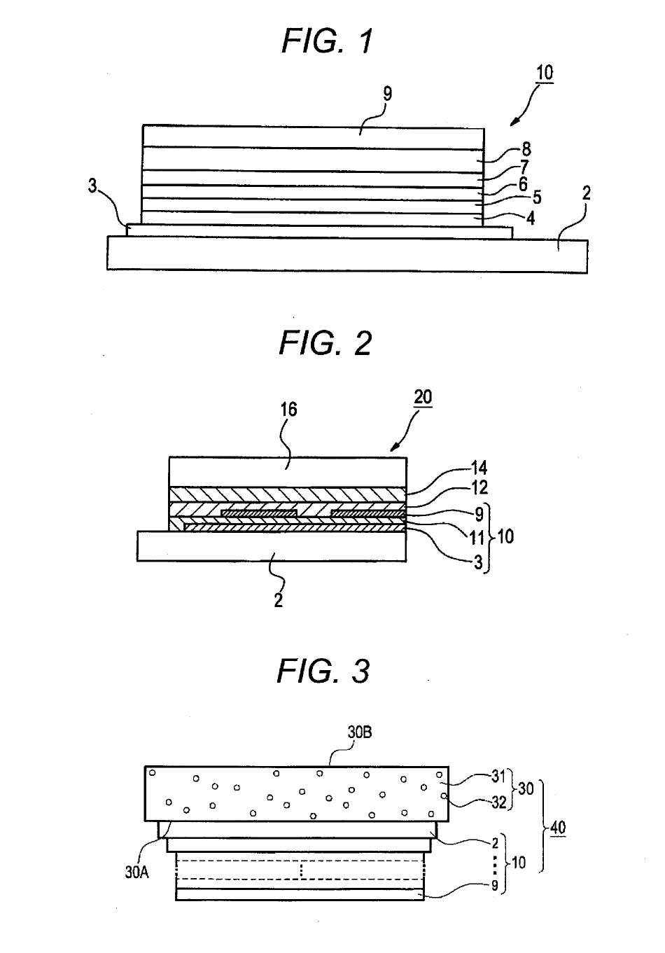

An example of the formation of the organic electroluminescence device according tow the invention is shown in FIG. 1. Organic electroluminescence device 10 in FIG. 1 has organic layers including light-emitting layer 6 between a pair of electrodes (anode 3 and cathode 9) on substrate 2. As organic layers, hole-injecting layer 4, hole-transporting layer 5, light-emitting layer 6, hole-blocking layer 7 and electron-transporting layer 8 are laminated in this order from the side of anode 3.

The formation of the device, substrate, anode and cathode of an organic electroluminescence device are described, for example, in JP-A-2008-270736, and the items described therein can be applied to the invention.

### [Light-Emitting Layer]

The light-emitting layer is a layer having functions to receive, at the time of electric field application, holes from the anode, hole-injecting layer or hole transporting layer, and receive electrons from the cathode, electron-injecting layer or electron-transporting layer, and offer the field of recombination of holes and electrons to emit light.

### <Light-Emitting Material>

Fluorescent materials and phosphorescent materials can be used in the invention as light-emitting materials and both materials may be used in combination.

These fluorescent materials and phosphorescent materials are described, for example, in JP-A-2008-270736, paragraphs [0100] to [0164] and JP-A-2007-266458, paragraphs [0088] to [0090], and the items described therein can be applied to the invention.

From the aspect of light emission efficiency, phosphorescent materials are preferred. As a preferred material of the phosphorescent material, a platinum complex represented by the following formula (C-1) can be exemplified.

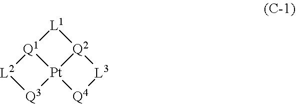

In formula (C-1), each of Q¹, Q², Q³ and Q⁴ independently represents a ligand to coordinate to Pt. Each of L¹, L² and L³ independently represents a single bond or a divalent linking group.

Formula (C-1) is described below.

In the first place, substituent group A and substituent group B are defined as follows.

### (Substituent Group A)

The examples of substituent group A include an alkyl group (preferably having 1 to 30 carbon atoms, more preferably 1 to 20 carbon atoms, and especially preferably 1 to 10 carbon atoms, e.g., methyl, ethyl, isopropyl, tert-butyl, n-octyl, n-decyl, n-hexadecyl, cyclopropyl, cyclopentyl, cyclohexyl and the like are exemplified), an alkenyl group (preferably having 2 to 30 carbon atoms, more preferably 2 to 20 carbon atoms, and especially preferably 2 to 10 carbon atoms, e.g., vinyl, allyl, 2-butenyl, 3-pentenyl and the like are exemplified), an alkynyl group (preferably having 2 to 30 carbon atoms, more preferably 2 to 20 carbon atoms, and especially preferably 2 to 10 carbon atoms, e.g., propargyl, 3-pentynyl and the like are exemplified), an aryl group (preferably having 6 to 30 carbon atoms, more preferably 6 to 20 carbon atoms, and especially preferably 6 to 12 carbon atoms, e.g., phenyl, p-methylphenyl, naphthyl, anthranyl and the like are exemplified), an amino group (preferably having 0 to 30 carbon atoms, more preferably 0 to 20 carbon atoms, and especially preferably 0 to 10 carbon atoms, e.g., amino, methylamino, dimethylamino, diethylamino, dibenzylamino, diphenylamino, ditolylamino and the like are exemplified), an alkoxy group (preferably having 1 to 30 carbon atoms, more preferably 1 to 20 carbon atoms, and especially preferably 1 to 10 carbon atoms, e.g., methoxy, ethoxy, butoxy, 2-ethylhexyloxy and the like are exemplified), an aryloxy group (preferably having 6 to 30 carbon atoms, more preferably 6 to 20 carbon atoms, and especially preferably 6 to 12 carbon atoms, e.g., phenyloxy, 1-naphthyloxy, 2-naphthyloxy and the like are exemplified), a heterocyclic oxy group (preferably having 1 to 30 carbon atoms, more preferably 1 to 20 carbon atoms, and especially preferably 1 to 12 carbon atoms, e.g., pyridyloxy, pyrazyloxy, pyrimidyloxy, quinolyloxy and the like are exemplified), an acyl group (preferably having 2 to 30 carbon atoms, more preferably 2 to 20 carbon atoms, and especially preferably 2 to 12 carbon atoms, e.g., acetyl, benzoyl, formyl, pivaloyl and the like are exemplified), an alkoxycarbonyl group (preferably having 2 to 30 carbon atoms, more preferably 2 to 20 carbon atoms, and especially preferably 2 to 12 carbon atoms, e.g., methoxycarbonyl, ethoxycarbonyl and the like are exemplified), an aryloxycarbonyl group (preferably having 7 to 30 carbon atoms, more preferably 7 to 20 carbon atoms, and especially preferably 7 to 12 carbon atoms, e.g., phenyloxycarbonyl and the like are exemplified), an acyloxy group (preferably having 2 to 30 carbon atoms, more preferably 2 to 20 carbon atoms, and especially preferably 2 to 10 carbon atoms, e.g., acetoxy, benzoyloxy and the like are exemplified), an acylamino group (preferably having 2 to 30 carbon atoms, more preferably 2 to 20 carbon atoms, and especially preferably 2 to 10 carbon atoms, e.g., acetylamino, benzoylamino and the like are exemplified), an alkoxycarbonylamino group (preferably having 2 to 30 carbon atoms, more preferably 2 to 20 carbon atoms, and especially preferably 2 to 12 carbon atoms, e.g., methoxycarbonylamino and the like are exemplified), an aryloxycarbonylamino group (preferably having 7 to 30 carbon atoms, more preferably 7 to 20 carbon atoms, and especially preferably 7 to 12 carbon atoms, e.g., phenyloxycarbonylamino and the like are exemplified), a sulfonylamino group (preferably having 1 to 30 carbon atoms, more preferably 1 to 20 carbon atoms, and especially preferably 1 to 12 carbon atoms, e.g., methanesulfonylamino, benzenesulfonylamino and the like are exemplified), a sulfamoyl group (preferably having 0 to 30 carbon atoms, more preferably 0 to 20 carbon atoms, and especially preferably 0 to 12 carbon atoms, e.g., sulfamoyl, methylsulfamoyl, dimethylsulfamoyl, phenylsulfamoyl and the like are exemplified), a carbamoyl group (preferably having 1 to 30 carbon atoms, more preferably 1 to 20 carbon atoms, and especially preferably 1 to 12 carbon atoms, e.g., carbamoyl, methylcarbamoyl, diethylcarbamoyl, phenylcarbamoyl and the like are exemplified), an alkylthio group (preferably having 1 to 30 carbon atoms, more preferably 1 to 20 carbon atoms, and especially preferably 1 to 12 carbon atoms, e.g., methylthio, ethylthio and the like are exemplified), an arylthio group (preferably having 6 to 30 carbon atoms, more preferably 6 to 20 carbon atoms, and especially preferably 6 to 12 carbon atoms, e.g., phenylthio and the like are exemplified), a heterocyclic thio group (preferably having 1 to 30 carbon atoms, more preferably 1 to 20 carbon atoms, and especially preferably 1 to 12 carbon atoms, e.g., pyridylthio, 2-benzimidazolylthio, 2-benzoxazolylthio, 2-benzothiazolylthio and the like are exemplified), a sulfonyl group (preferably having 1 to 30 carbon atoms, more preferably 1 to 20 carbon atoms, and especially preferably 1 to 12 carbon atoms, e.g., mesyl, tosyl and the like are exemplified), a sulfinyl group (preferably having 1 to 30 carbon atoms, more preferably 1 to 20 carbon atoms, and especially preferably 1 to 12 carbon atoms, e.g., methanesulfinyl, benzenesulfinyl and the like are exemplified), a ureido group (preferably having 1 to 30 carbon atoms, more preferably 1 to 20 carbon atoms, and especially preferably 1 to 12 carbon atoms, e.g., ureido, methylureido, phenylureido and the like are exemplified), a phosphoric amide group (preferably having 1 to 30 carbon atoms, more preferably 1 to 20 carbon atoms, and especially preferably 1 to 12 carbon atoms, e.g., diethylphosphoric amide, phenylphosphoric amide and the like are exemplified), a hydroxyl group, a mercapto group, a halogen atom (e.g., a fluorine atom, a chlorine atom, a bromine atom, and an iodine atom), a cyano group, a sulfo group, a carboxyl group, a nitro group, a hydroxamic acid group, a sulfino group, a hydrazino group, an imino group, a heterocyclic group (also including an aromatic heterocyclic group and preferably having 1 to 30 carbon atoms, and more preferably 1 to 12 carbon atoms, the examples of the hetero atoms include e.g., a nitrogen atom, an oxygen atom, a sulfur atom, a phosphorus atom, a silicon atom, a selenium atom, and a tellurium atom, and specifically pyridyl, pyrazyl, pyrimidyl, pyridazinyl, pyrrolyl, pyrazolyl, triazolyl, imidazolyl, oxazolyl, thiazolyl, isoxazolyl, isothiazolyl, quinolyl, furyl, thienyl, selenophenyl, tellurophenyl, piperidyl, piperidino, morpholino, pyrrolidyl, pyrrolidino, benzoxazolyl, benzimidazolyl, benzothiazolyl, a carbazolyl group, an azepinyl group, a silolyl group and the like are exemplified), a silyl group (preferably having 3 to 40 carbon atoms, more preferably 3 to 30 carbon atoms, and especially preferably 3 to 24 carbon atoms, e.g., trimethylsilyl, triphenylsilyl and the like are exemplified), a silyloxy group (preferably having 3 to 40 carbon atoms, more preferably 3 to 30 carbon atoms, and especially preferably 3 to 24 carbon atoms, e.g., trimethylsilyloxy, triphenylsilyloxy and the like are exemplified), and a phosphoryl group (e.g., a diphenylphosphoryl group, a dimethylphosphoryl group and the like are exemplified).

These substituents may further be substituted, and as further substituents, the groups selected from the above substituent group A can be exemplified.

### (Substituent Group B)

The examples of substituent group B include an alkyl group (preferably having 1 to 30 carbon atoms, more preferably 1 to 20 carbon atoms, and especially preferably 1 to 10 carbon atoms, e.g., methyl, ethyl, isopropyl, tert-butyl, n-octyl, n-decyl, n-hexadecyl, cyclopropyl, cyclopentyl, cyclohexyl and the like are exemplified), an alkenyl group (preferably having 2 to 30 carbon atoms, more preferably 2 to 20 carbon atoms, and especially preferably 2 to 10 carbon atoms, e.g., vinyl, allyl, 2-butenyl, 3-pentenyl and the like are exemplified), an alkynyl group (preferably having 2 to 30 carbon atoms, more preferably 2 to 20 carbon atoms, and especially preferably 2 to 10 carbon atoms, e.g., propargyl, 3-pentynyl and the like are exemplified), an aryl group (preferably having 6 to 30 carbon atoms, more preferably 6 to 20 carbon atoms, and especially preferably 6 to 12 carbon atoms, e.g., phenyl, p-methylphenyl, naphthyl, anthranyl and the like are exemplified), a cyano group, and a heterocyclic group (also including an aromatic heterocyclic group and preferably having 1 to 30 carbon atoms, and more preferably 1 to 12 carbon atoms, the examples of the hetero atoms include e.g., a nitrogen atom, an oxygen atom, a sulfur atom, a phosphorus atom, a silicon atom, a selenium atom, and a tellurium atom, and specifically pyridyl, pyrazyl, pyrimidyl, pyridazinyl, pyrrolyl, pyrazolyl, triazolyl, imidazolyl, oxazolyl, thiazolyl, isoxazolyl, isothiazolyl, quinolyl, furyl, thienyl, selenophenyl, tellurophenyl, piperidyl, piperidino, morpholino, pyrrolidyl, pyrrolidino, benzoxazolyl, benzimidazolyl, benzothiazolyl, a carbazolyl group, an azepinyl group, a silolyl group and the like are exemplified). These substituents may further be substituted, and as further substituents, the groups selected from the above substituent groups A and B can be exemplified.

In the invention, “carbon atom number” of the above substituents such as an alkyl group and the like also includes the case where substituents such as an alkyl group and the like may further be substituted with other substituent, that is, “carbon atom number” is used in the meaning of including the carbon atom number of other substituent also.

In formula (C-1), each of Q¹, Q², Q³ and Q⁴ independently represents a ligand to coordinate to Pt. At this time, bonding of Q¹, Q², Q³ and Q⁴ to Pt may be any of a covalent bond, an ionic bond, and a coordinate bond. The atoms in Q¹, Q², Q³ and Q⁴ to bond to Pt are preferably a carbon atom, a nitrogen atom, an oxygen atom, a sulfur atom and a phosphorus atom, and it is preferred that at least one of the atoms in Q¹, Q², Q³ and Q⁴ to bond to Pt is a carbon atom, it is more preferred that two of these atoms are carbon atoms, and that two are carbon atoms and two are nitrogen atoms is especially preferred.

Q¹, Q², Q³ and Q⁴ to bond to Pt via a carbon atom may be an anionic ligand or a neutral ligand. As the anionic ligands, a vinyl ligand, an aromatic hydrocarbon ring ligand (e.g., a benzene ligand, a naphthalene ligand, an anthracene ligand, a phenanthrene ligand, etc.), a heterocyclic ligand (e.g., a furan ligand, a thiophene ligand, a pyridine ligand, a pyrazine ligand, a pyrimidine ligand, a pyridazine ligand, a triazine ligand, a thiazole ligand, an oxazole ligand, a pyrrole ligand, an imidazole ligand, a pyrazole ligand, a triazole ligand, and condensed ring products containing these ligands (e.g., a quinoline ligand, a benzothiazole ligand, etc.)) are exemplified. As the neutral ligand, a carbene ligand is exemplified.

Q¹, Q², Q³ and Q⁴ to bond to Pt via a nitrogen atom may be a neutral ligand or an anionic ligand. As the neutral ligands, a nitrogen-containing aromatic heterocyclic ligand (a pyridine ligand, a pyrazine ligand, a pyrimidine ligand, a pyridazine ligand, a triazine ligand, an imidazole ligand, a pyrazole ligand, a triazole ligand, an oxazole ligand, a thiazole ligand, and condensed ring products containing these ligands (e.g., a quinoline ligand, a benzimidazole ligand, etc.)), an amine ligand, a nitrile ligand, and an imine ligand are exemplified. As the anionic ligands, an amino ligand, an imino ligand, a nitrogen-containing aromatic heterocyclic ligand (e.g., a pyrrole ligand, an imidazole ligand, a triazole ligand, and condensed ring products containing these ligands (e.g., an indole ligand, a benzimidazole ligand, etc.)) are exemplified.

Q¹, Q², Q³ and Q⁴ to bond to Pt via an oxygen atom may be a neutral ligand or an anionic ligand. As the neutral ligands, an ether ligand, a ketone ligand, an ester ligand, an amido ligand, an oxygen-containing heterocyclic ligand (e.g., a furan ligand, an oxazole ligand, and condensed ring products containing these ligands (e.g., a benzoxazole ligand, etc.)) are exemplified. As the anionic ligands, an alkoxy ligand, an aryloxy ligand, an aromatic heterocyclic oxy ligand, an acyloxy ligand, a silyloxy ligand, etc., are exemplified.

Q¹, Q², Q³ and Q⁴ to bond to Pt via a sulfur atom may be a neutral ligand or an anionic ligand. As the neutral ligands, a thioether ligand, a thioketone ligand, a thioester ligand, a thioamide ligand, a sulfur-containing heterocyclic ligand (e.g., a thiophene ligand, a thiazole ligand, and condensed ring products containing these ligands (e.g., a benzothiazole ligand, etc.)) are exemplified. As the anionic ligands, an alkylmercapto ligand, an arylmercapto ligand, an aromatic heterocyclic mercapto ligand, etc., are exemplified.

Q¹, Q², Q³ and Q⁴ to bond to Pt via a phosphorus atom may be a neutral ligand or an anionic ligand. As the neutral ligands, a phosphine ligand, a phosphoric ester ligand, a phosphorous ester ligand, a phosphorus-containing ligand (e.g., a phosphinine ligand, etc.) are exemplified. As the anionic ligands, a phosphino ligand, a phosphinyl ligand, a phosphoryl ligand are exemplified.

Each of the groups represented by Q¹, Q², Q³ and Q⁴ may have a substituent, and as the substituents, those exemplified above as substituent group A can be arbitrarily applied. In addition, substituents may be linked to each other (when Q³ and Q⁴ are linked, the Pt complex is a Pt complex of a cyclic tetradentate ligand).

The groups represented by each of Q¹, Q², Q³ and Q⁴ are preferably an aromatic hydrocarbon ring ligand to bond to Pt via a carbon atom, an aromatic heterocyclic ligand to bond to Pt via a carbon atom, a nitrogen-containing aromatic heterocyclic ligand to bond to Pt via a nitrogen atom, an acyloxy ligand, an alkyloxy ligand, an aryloxy ligand, an aromatic heterocyclic oxy ligand, and a silyloxy ligand, more preferably an aromatic hydrocarbon ring ligand to bond to Pt via a carbon atom, an aromatic heterocyclic ligand to bond to Pt via a carbon atom, a nitrogen-containing aromatic heterocyclic ligand to bond to Pt via a nitrogen atom, an acyloxy ligand, and an aryloxy ligand, and still more preferably an aromatic hydrocarbon ring ligand to bond to Pt via a carbon atom, an aromatic heterocyclic ligand to bond to Pt via a carbon atom, a nitrogen-containing aromatic heterocyclic ligand to bond to Pt via a nitrogen atom, and an acyloxy ligand.

Each of L¹, L² and L³ represents a single bond or a divalent linking group. As the divalent linking groups represented by L¹, L² and L³, an alkylene group (e.g., methylene, ethylene, propylene, etc.), an arylene group (e.g., phenylene, naphthalenediyl), a hetero-arylene group (e.g., pyridinediyl, thiophenediyl, etc.), an imino group (—NR—) (e.g., a phenylimino group, etc.), an oxy group (—O—), a thio group (—S—), a phosphinidene group (—PR—) (e.g., a phenylphosphinidene group, etc.), a silylene group (—SiRR′—) (e.g., a dimethylsilylene group, a diphenylsilylene group, etc.), and groups obtained by combining these groups are exemplified (each of R and R′ represents a substituent).

Each of these divalent linking groups may further have a substituent. As the substituents, an alkyl group and an aryl group are exemplified. When two or more substituents are present, they may be bonded to each other to form a ring. In the case where the substituent is an alkyl group, preferred alkyl groups are a methyl group, an ethyl group, a propyl group, an i-butyl group, a t-butyl group, a trifluoromethyl group, and a group forming a cyclohexyl group or a cyclopentyl group by bonding to each other. In the case where the substituent is an aryl group, preferred aryl groups are a phenyl group and a group forming a fluorene group by bonding to each other. The most preferred substituents are a methyl group, an ethyl group, a propyl group and an i-butyl group.

From the viewpoints of stability of the complex and yield of light emitting quantum, each of L¹, L² and L³ preferably represents a single bond, an alkylene group, an arylene group, a hetero-arylene group, an imino group, an oxy group, a thio group, or a silylene group, more preferably a single bond, an alkylene group, an arylene group, or an imino group, still more preferably a single bond, an alkylene group, or an arylene group, still further preferably a single bond, a methylene group, or a phenylene group, still yet more preferably a single bond, a di-substituted methylene group, still yet further preferably a single bond, a dimethylmethylene group, a diethylmethylene group, a diisobutylmethylene group, a dibenzylmethylene group, an ethylmethylmethylene group, a methylpropylmethylene group, an isobutylmethylmethylene group, a diphenylmethylene group, a methylphenylmethylene group, a cyclohexanediyl group, a cyclopentanediyl group, a fluorenediyl group, or a fluoromethylmethylene group, and especially preferably represents a single bond, a dimethylmethylene group, a diphenylmethylene group, or a cyclohexanediyl group.

The platinum complex represented by formula (C-1) is more preferably represented by the following formula (C-2).

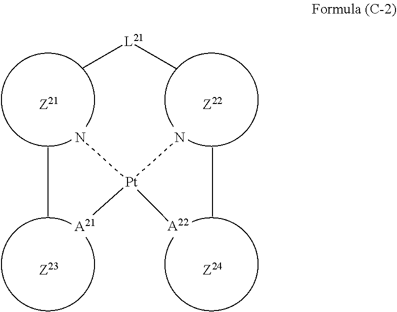

In formula (C-2), L²¹ represents a single bond or a divalent linking group. Each of A²¹ and A²² independently represents C or N. Each of Z²¹ and Z²² independently represents a nitrogen-containing aromatic heterocyclic ring. Each of Z²³ and Z²⁴ independently represents a benzene ring or an aromatic heterocyclic ring.

Formula (C-2) will be described below. L²¹ has the same meaning with L¹ in formula (C-1) and the preferred range is also the same.

Each of A²¹ and A²² independently represents a carbon atom or a nitrogen atom. At least one of A²¹ and A²² preferably represents a carbon atom, and from the viewpoints of stability and yield of light emitting quantum of the complex, it is preferred that both A²¹ and A²² represent a carbon atom.

Each of Z²¹ and Z²² independently represents a nitrogen-containing aromatic heterocyclic ring. As the nitrogen-containing aromatic heterocyclic ring represented by Z²¹ and Z²², a pyridine ring, a pyrimidine ring, a pyrazine ring, a triazine ring, an imidazole ring, a pyrazole ring, an oxazole ring, a thiazole ring, a triazole ring, an oxadiazole ring, and a thiadiazole ring are exemplified. From the viewpoints of stability of the complex, control of light emission wavelength and yield of light emitting quantum of the complex, the rings represented by Z²¹ and Z²² are preferably a pyridine ring, a pyrazine ring, an imidazole ring, and a pyrazole ring, more preferably a pyridine ring, an imidazole ring, and a pyrazole ring, still more preferably a pyridine ring and a pyrazole ring, and especially preferably a pyridine ring.

The nitrogen-containing aromatic heterocyclic ring represented by each of Z²¹ and Z²² may have a substituent, and as the substituent on the carbon atom, those exemplified above as substituent group A can be applied, and as the substituent on the nitrogen atom, those exemplified above as substituent group B can be applied. Preferred substituents on the carbon atom include an alkyl group, a perfluoroalkyl group, an aryl group, an aromatic heterocyclic group, a dialkylamino group, a diarylamino group, an alkoxy group, a cyano group, and a halogen atom. Substituents are arbitrarily selected for the purpose of controlling light emission wavelength and electric potential. In the case of shortening the wavelength, an electron-donating group, a fluorine atom and an aromatic ring group are preferred and, for example, an alkyl group, a dialkylamino group, an alkoxy group, a fluorine atom, an aryl group, and an aromatic heterocyclic group are selected. In the case of lengthening the wavelength, an electron-withdrawing group is preferred and, for example, a cyano group and a perfluoroalkyl group are selected. Preferred substituents on the nitrogen atom include an alkyl group, an aryl group and an aromatic heterocyclic group, and from the point of the stability of the complex, an alkyl group and an aryl group are preferred. The substituents may be linked to each other to form a condensed ring, and as the rings to be formed, a benzene ring, a pyridine ring, a pyrazine ring, a pyridazine ring, a pyrimidine ring, an imidazole ring, an oxazole ring, a thiazole ring, a pyrazole ring, a thiophene ring, and a furan ring are exemplified.

Each of Z²³ and Z²⁴ independently represents a benzene ring or an aromatic heterocyclic ring. As the nitrogen-containing aromatic heterocyclic ring represented by Z²³ and Z²⁴, a pyridine ring, a pyrimidine ring, a pyrazine ring, a pyridazine ring, a triazine ring, an imidazole ring, a pyrazole ring, an oxazole ring, a thiazole ring, a triazole ring, an oxadiazole ring, a thiadiazole ring, a thiophene ring and a furan ring are exemplified. From the viewpoints of stability of the complex, control of light emission wavelength and yield of light emitting quantum of the complex, the rings represented by Z²³ and Z²⁴ are preferably a benzene ring, a pyridine ring, a pyrazine ring, an imidazole ring, a pyrazole ring, and a thiophene ring, more preferably a benzene ring, a pyridine ring, and a pyrazole ring, and still more preferably a benzene ring and a pyridine ring.

The benzene ring and nitrogen-containing aromatic heterocyclic ring represented by each of Z²³ and Z²⁴ may have a substituent, and as the substituent on the carbon atom, those exemplified above as substituent group A can be applied, and as the substituent on the nitrogen atom, those exemplified above as substituent group B can be applied. Preferred substituents on the carbon atom include an alkyl group, a perfluoroalkyl group, an aryl group, an aromatic heterocyclic group, a dialkylamino group, a diarylamino group, an alkoxy group, a cyano group, and a halogen atom. Substituents are arbitrarily selected for the purpose of controlling light emission wavelength and electric potential. In the case of lengthening the wavelength, an electron-donating group and an aromatic ring group are preferred and, for example, an alkyl group, a dialkylamino group, an alkoxy group, an aryl group and an aromatic heterocyclic group are selected. In the case of shortening the wavelength, an electron-withdrawing group is preferred and, for example, a fluorine group, a cyano group and a perfluoroalkyl group are selected. Preferred substituents on the nitrogen atom include an alkyl group, an aryl group and an aromatic heterocyclic group, and from the point of the stability of the complex, an alkyl group and an aryl group are preferred. The substituents may be linked to each other to form a condensed ring, and as the rings to be formed, a benzene ring, a pyridine ring, a pyrazine ring, a pyridazine ring, a pyrimidine ring, an imidazole ring, an oxazole ring, a thiazole ring, a pyrazole ring, a thiophene ring, and a furan ring are exemplified.

Of the platinum complexes represented by formula (C-2), one of more preferred embodiments is a platinum complex represented by the following formula (C-3).

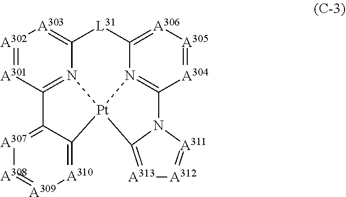

In formula (C-3), each of A³⁰¹ to A³¹³ independently represents C—R or N. R represents a hydrogen atom or a substituent. L³¹ represents a single bond or a divalent linking group.

Formula (C-3) will be described below. L³¹ has the same meaning with L²¹ in formula (C-2) and the preferred range is also the same. Each of A³⁰¹ to A³⁰⁶ independently represents C—R or N. R represents a hydrogen atom or a substituent. As the substituents represented by R, those exemplified above as substituent group A can be applied.

Each of A³⁰¹ to A³⁰⁶ preferably represents C—R, and R may be linked to each other to form a ring. When each of A³⁰¹ to A³⁰⁶ represents C—R, R of A³⁰² and A³⁰⁵ is preferably a hydrogen atom, an alkyl group, an aryl group, an amino group, an alkoxy group, an aryloxy group, a fluorine group or a cyano group, more preferably a hydrogen atom, an amino group, an alkoxy group, an aryloxy group, or a fluorine group, and especially preferably a hydrogen atom or a fluorine group. R of A³⁰¹, A³⁰⁴ and A³⁰⁶ is preferably a hydrogen atom, an alkyl group, an aryl group, an amino group, an alkoxy group, an aryloxy group, a fluorine group, or a cyano group, more preferably a hydrogen atom, an amino group, an alkoxy group, an aryloxy group, or a fluorine group, and especially preferably a hydrogen atom. Each of A³⁰⁷, A³⁰⁸, A³⁰⁹ and A³¹⁰ independently represents C—R or N. R represents a hydrogen atom or a substituent. As the substituents represented by R, those exemplified above as substituent group A can be applied. When each of A³⁰⁷, A³⁰⁸, A³⁰⁹ and A³¹⁰ represents C—R, R preferably represents a hydrogen atom, an alkyl group, a perfluoroalkyl group, an aryl group, an aromatic heterocyclic group, a dialkylamino group, a diarylamino group, an alkyloxy group, a cyano group, or a halogen atom, more preferably a hydrogen atom, an alkyl group, a perfluoroalkyl group, an aryl group, a dialkylamino group, a cyano group, or a fluorine atom, and still more preferably a hydrogen atom, an alkyl group, a trifluoromethyl group, or a fluorine atom. Further, if possible, substituents may be linked to each other to form a condensed ring structure. When light emission wavelength is shifted to the shorter wave side, it is preferred that A³⁰⁸ represents an N atom.

When each of A³⁰⁷, A³⁰⁸, A³⁰⁹ and A³¹⁰ is selected as described above, as the 6-membered ring formed by two carbon atoms and A³⁰⁷, A³⁰⁸, A³⁰⁹, A³¹⁰, a benzene ring, a pyridine ring, a pyrazine ring, a pyrimidine ring, a pyridazine ring and a triazine ring are exemplified, more preferably a benzene ring, a pyridine ring, a pyrazine ring, a pyrimidine ring, and a pyridazine ring are exemplified, and especially preferably a benzene ring or a pyridine ring. By the fact that the above 6-membered ring is a pyridine ring, a pyrazine ring, a pyrimidine ring, or a pyridazine ring (especially preferably a pyridine ring), as compared with a benzene ring, acidicity of the hydrogen atom present at the position for forming a metal-carbon bond is improved, and so a metal complex is easily formed, therefore, advantageous.

Each of A³¹¹, A³¹² and A³¹³ independently represents C—R or N. R represents a hydrogen atom or a substituent. As the substituents represented by R, those exemplified above as substituent group A can be applied. When each of A³¹¹, A³¹² and A³¹³ represents C—R, R is preferably a hydrogen atom, an alkyl group, a perfluoroalkyl group, an aryl group, an aromatic heterocyclic group, a dialkylamino group, a diarylamino group, an alkyloxy group, a cyano group, or a halogen atom, more preferably a hydrogen atom, an alkyl group, a perfluoroalkyl group, an aryl group, a dialkylamino group, a cyano group, or a fluorine atom, and still more preferably a hydrogen atom, an alkyl group, a trifluoromethyl group, or a fluorine atom. If possible, substituents may be linked to each other to form a condensed ring structure. It is preferred that at least one of A³¹¹, A³¹² and A³¹³ represents N, and it is especially preferred that A³¹¹ represents N.

Of the platinum complexes represented by formula (C-2), one of more preferred embodiments is a platinum complex represented by the following formula (C-4).

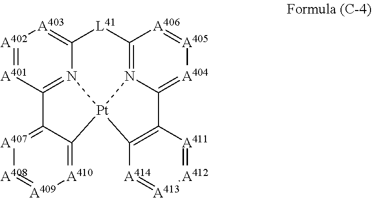

In formula (C-4), each of A⁴⁰¹ to A⁴¹⁴ independently represents C—R or N. R represents a hydrogen atom or a substituent. L⁴¹ represents a single bond or a divalent linking group.

Formula (C-4) is described below.

Each of A⁴⁰¹ to A⁴¹⁴ independently represents C—R or N. R represents a hydrogen atom or a substituent. Each of A⁴⁰¹ to A⁴⁰⁶ and L⁴¹ has the same meaning with A³⁰¹ to A³⁰⁶ and L³¹ in formula (C-3) and the preferred range is also the same.

Concerning A⁴⁰⁷ to A⁴¹⁴, the number of N (a nitrogen atom) is preferably 0 to 2, and more preferably 0 or 1, in each of A⁴⁰⁷ to A⁴¹⁰ and A⁴¹¹ to A⁴¹⁴. When light emission wavelength is shifted to the shorter wave side, it is preferred that A⁴⁰⁸ or A⁴¹² represents an N atom, and more preferably both A⁴⁰⁸ and A⁴¹² represent an N atom.

When each of A⁴⁰⁷ to A⁴¹⁴ represents C—R, R of A⁴⁰⁸ and A⁴¹² is preferably a hydrogen atom, an alkyl group, a perfluoroalkyl group, an aryl group, an amino group, an alkoxy group, an aryloxy group, a fluorine group, or a cyano group, more preferably a hydrogen atom, a perfluoroalkyl group, an alkyl group, an aryl group, a fluorine group, or a cyano group, and especially preferably a hydrogen atom, a phenyl group, a perfluoroalkyl group, or a cyano group. R of A⁴⁰⁷, A⁴⁰⁹, A⁴¹¹ and A⁴¹³ is preferably a hydrogen atom, an alkyl group, a perfluoroalkyl group, an aryl group, an amino group, an alkoxy group, an aryloxy group, a fluorine group, or a cyano group, more preferably a hydrogen atom, a perfluoroalkyl group, a fluorine group, or a cyano group, and especially preferably a hydrogen atom, a phenyl group, or a cyano group. R of A⁴¹⁰ and A⁴¹⁴ is preferably a hydrogen atom or a fluorine group, and more preferably a hydrogen atom. When either of A⁴⁰⁷ to A⁴⁰⁹ and A⁴¹¹ to A⁴¹³ represents C—R, R may be bonded to each other to form a ring.

Of the platinum complexes represented by formula (C-2), one of more preferred embodiments is a platinum complex represented by the following formula (C-5).

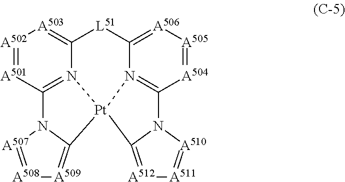

In formula (C-5), each of A⁵⁰¹ to A⁵¹² independently represents C—R or N. R represents a hydrogen atom or a substituent. L⁵¹ represents a single bond or a divalent linking group.

Formula (C5) is described below. Each of A⁵⁰¹ to A⁵⁰⁶ and L⁵¹ has the same meaning with A³⁰¹ to A³⁰⁶ and L³¹ in formula (C-3) and the preferred range is also the same.

Each of A⁵⁰⁷, A⁵⁰⁸ and A⁵⁰⁹, and A⁵¹⁰, A⁵¹¹ and A⁵¹² independently has the same meaning with A³¹¹, A³¹² and A³¹³ in formula (C-3) and the preferred range is also the same.

Of the platinum complexes represented by formula (C-1), one of more preferred embodiments is a platinum complex represented by the following formula (C-6).

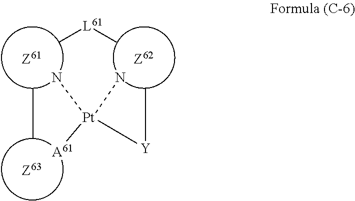

In formula (C-6), L⁶¹ represents a single bond or a divalent linking group. Each of A⁶¹ independently represents C or N. Each of Z⁶¹ and Z⁶² independently represents a nitrogen-containing aromatic heterocyclic ring.

Each of Z⁶³ independently represents a benzene ring or an aromatic heterocyclic ring. Y is an anionic acyclic ligand bonding to Pt.

Formula (C-6) will be described. L⁶¹ has the same meaning with L¹ in formula (C-1), and preferred range is also the same.

A⁶¹ represents C or N. From the viewpoints of stability of the complex and yield of light emitting quantum of the complex, A⁶¹ preferably represents C.

Z⁶¹ and Z⁶² have the same meaning with Z²¹ and Z²² in formula (C-2) respectively, and the preferred ranges are also the same. L⁶³ has the same meaning with L²³ in formula (C-2), and the preferred range is also the same.

Y is an anionic acyclic ligand bonding to Pt. An acyclic ligand is a ligand whose atoms bonding to Pt do not form a ring in the state of ligand. As the atoms in Y bonding to Pt, a carbon atom, a nitrogen atom, an oxygen atom and a sulfur atom are preferred, a nitrogen atom and an oxygen atom are more preferred and an oxygen atom is most preferred. As Y bonding to Pt via a carbon atom, a vinyl ligand is exemplified. As Y bonding to Pt via a nitrogen atom, an amino ligand and an imino ligand are exemplified. As Y bonding to Pt via an oxygen atom, an alkoxy ligand, an aryloxy ligand, an aromatic heterocyclic oxy ligand, an acyloxy ligand, a silyloxy ligand, a carboxyl ligand, a phosphoric acid ligand, and sulfonic acid ligand are exemplified. As Y bonding to Pt via a sulfur atom, an alkylmercapto ligand, an arylmercapto ligand, an aromatic heterocyclic mercapto ligand, and a thiocarboxylic acid ligand are exemplified.

The ligand represented by Y may have a substituent, and as the examples of the substituents, those exemplified above as substituent group A can be arbitrarily applied. Further, substituents may be linked to each other.

Of the ligands represented by Y, a ligand bonding to Pt via an oxygen atom is preferred, more preferred is an acyloxy ligand, an alkyloxy ligand, an aryloxy ligand, an aromatic heterocyclic oxy ligand, or a silyloxy ligand, and still more preferred is an acyloxy ligand.

Of the platinum complexes represented by formula (C-6), one of more preferred embodiments is a platinum complex represented by the following formula (C-7).

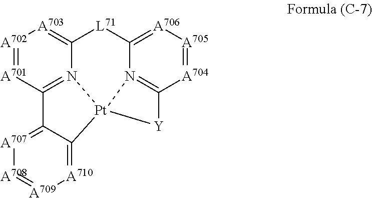

In formula (C-7), each of A⁷⁰¹ to A⁷¹⁰ independently represents C—R or N. R represents a hydrogen atom or a substituent. L⁷¹ represents a single bond or a divalent linking group. Y is an anionic acyclic ligand bonding to Pt.

Formula (C-7) will be described. L⁷¹ has the same meaning with L⁶¹ in formula (C-6), and preferred range is also the same. Each of A⁷⁰¹ to A⁷¹⁰ has the same meaning with A³⁰¹ to A³¹⁰ in formula (C-3) and the preferred range is also the same. Y has the same meaning with Y in formula (C-6) and the preferred range is also the same.

As the platinum complexes represented by formula (C-1), specifically the following compounds are exemplified. The compounds disclosed in JP-A-2005-310733, paragraphs [0143] to [0152], [0157] to [0158], and [0162] to [0168], the compounds disclosed in JP-A-2006-256999, paragraphs [0065] to [0083], the compounds disclosed in JP-A-2006-93542, paragraphs [0065] to [0090], the compounds disclosed in JP-A-2007-73891, paragraphs [0063] to [0071], the compounds disclosed in JP-A-2007-324309, paragraphs [0079] to [0083], the compounds disclosed in JP-A-2007-96255, paragraphs [0055] to [0071], and the compounds disclosed in JP-A-2006-313796, paragraphs [0043] to [0046]. In addition to the above compounds, the platinum complexes shown below are exemplified. In the following exemplification, Me means a methyl group.

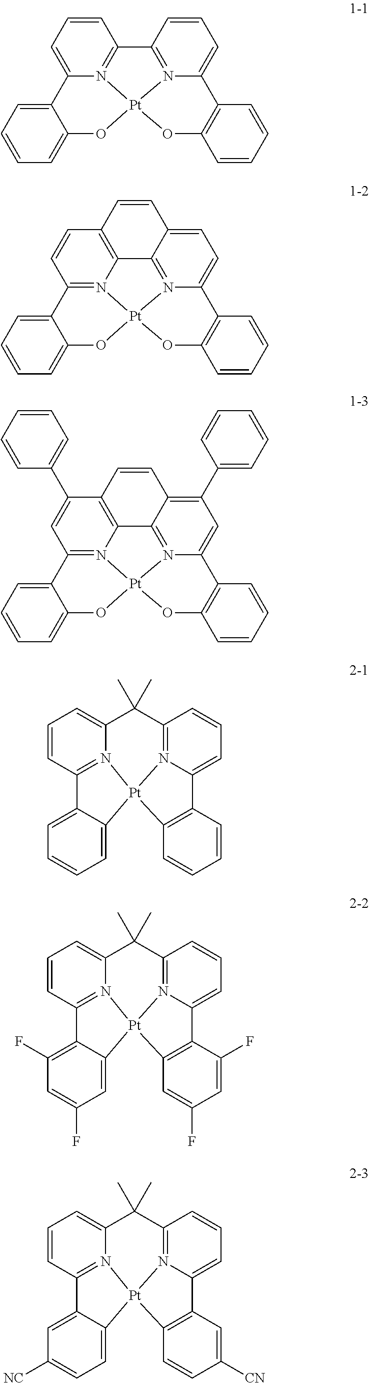

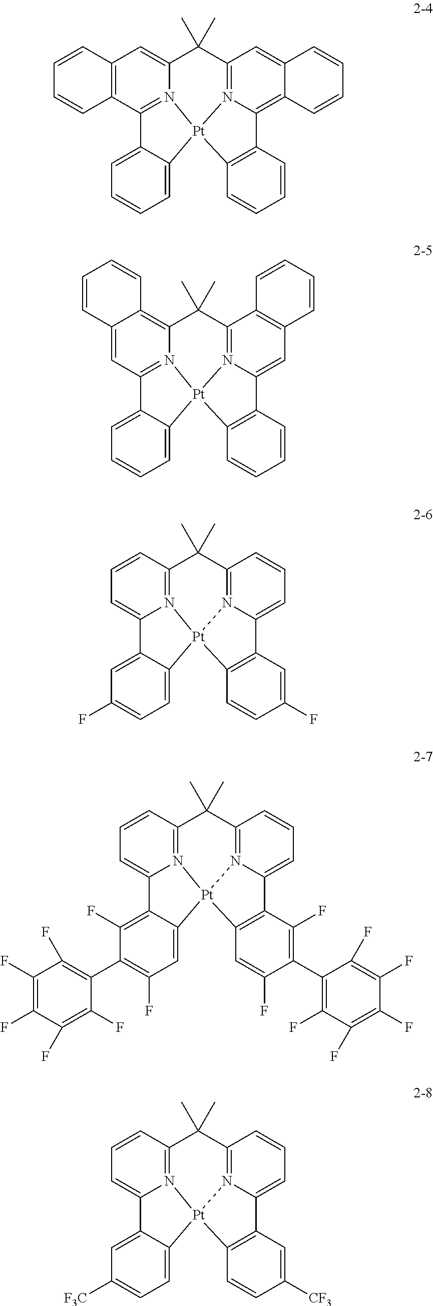

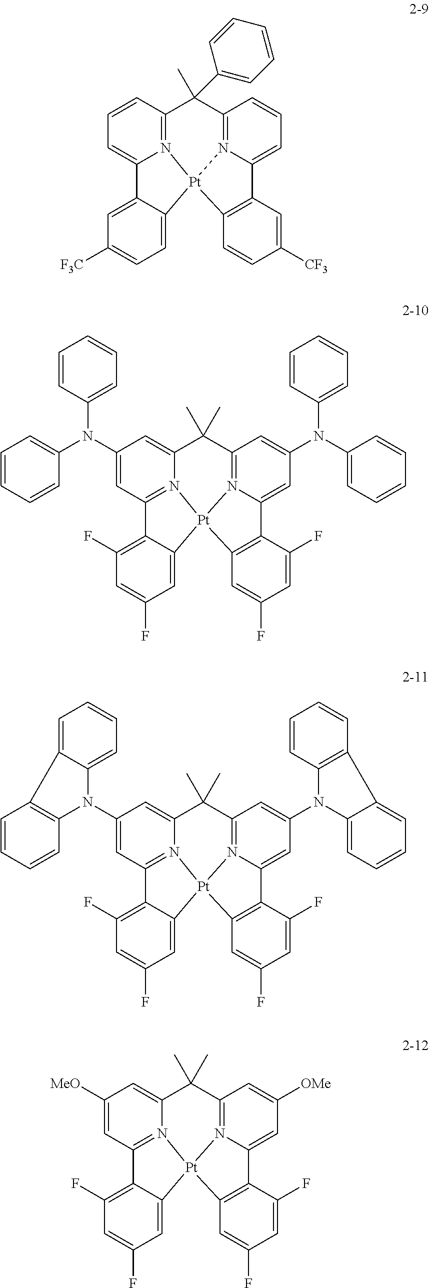

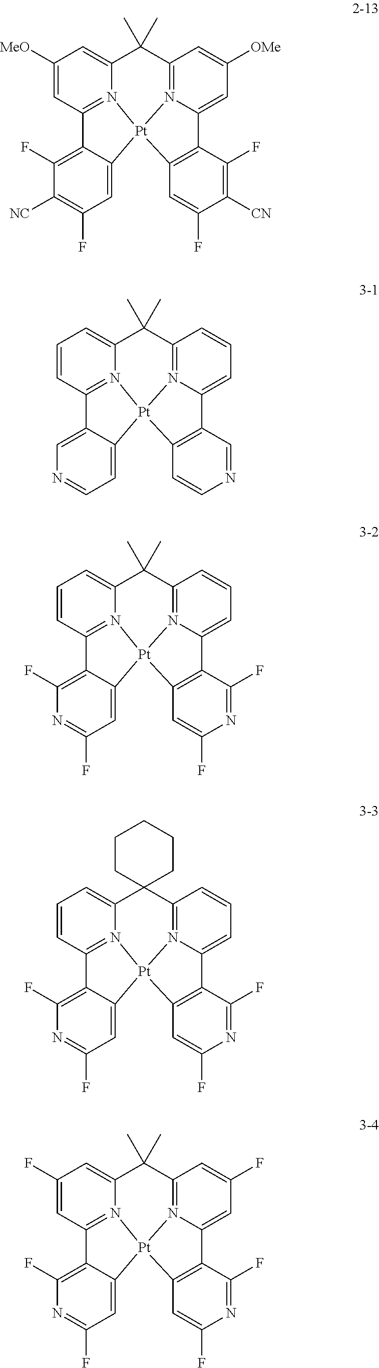

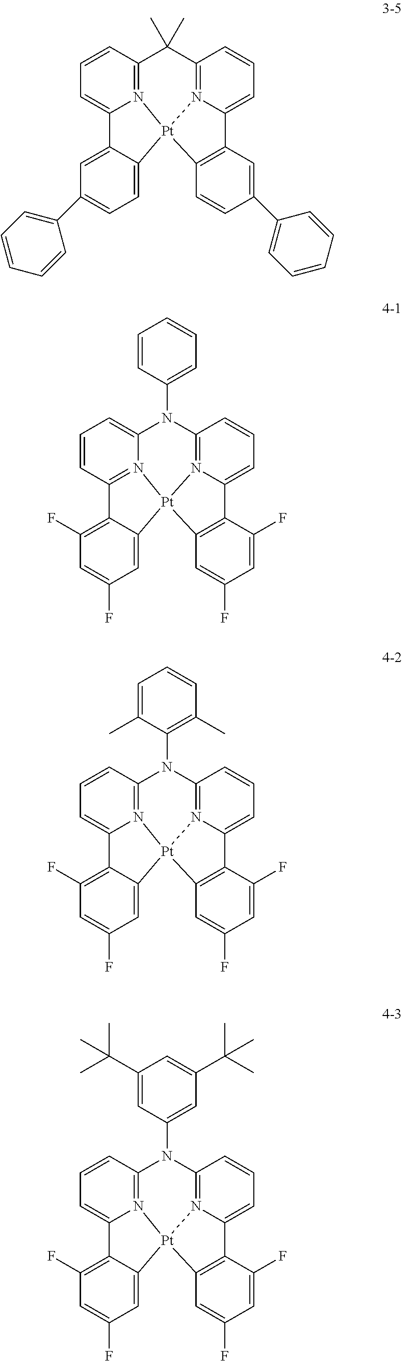

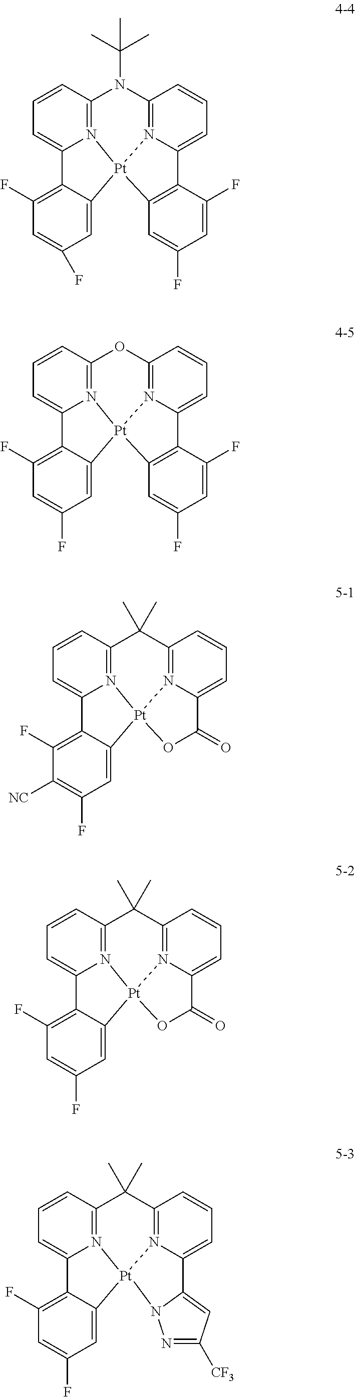

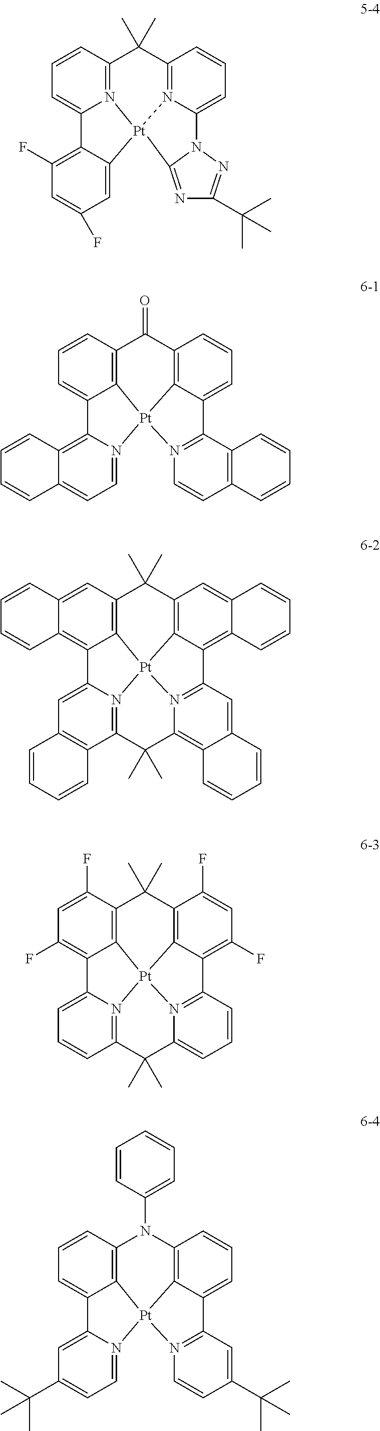

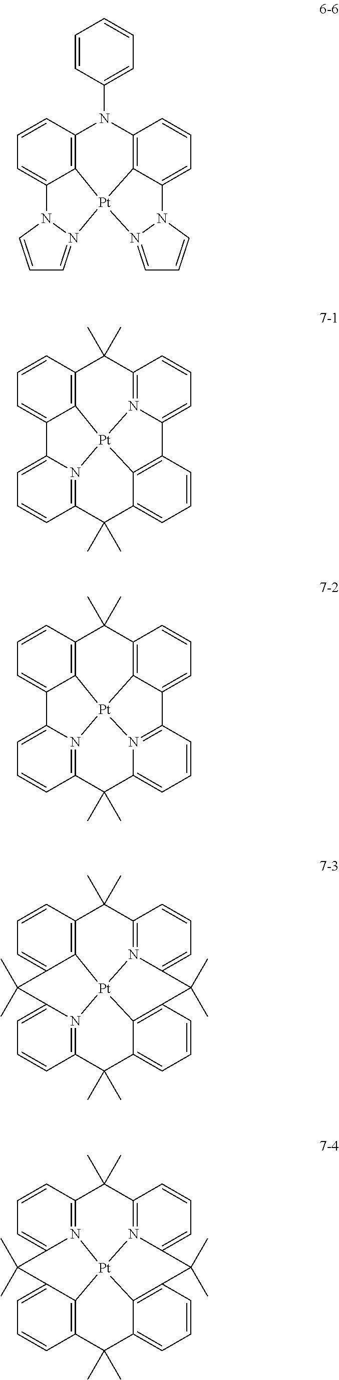

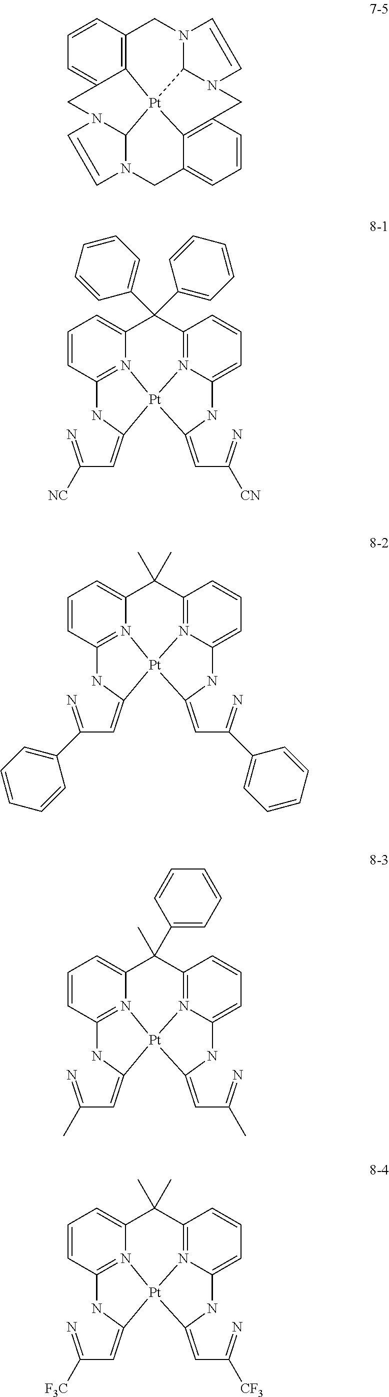

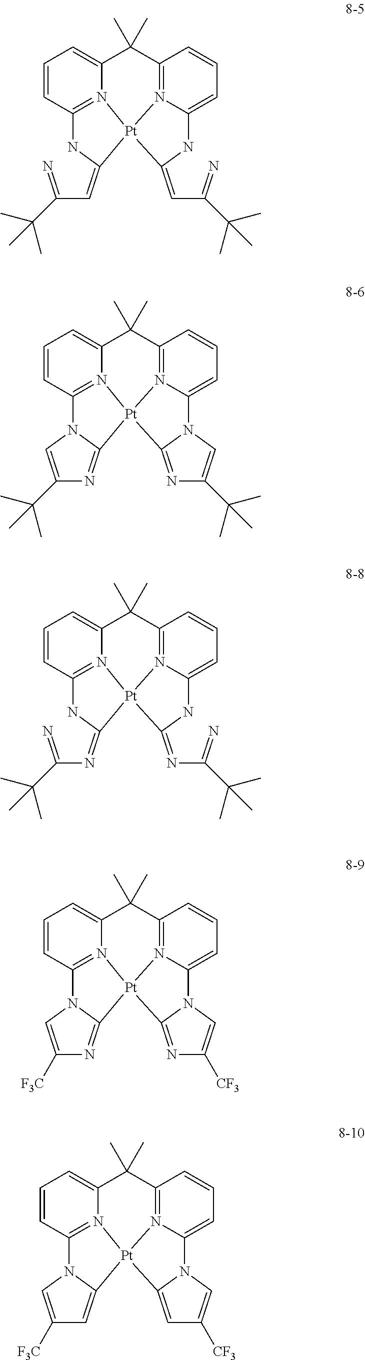

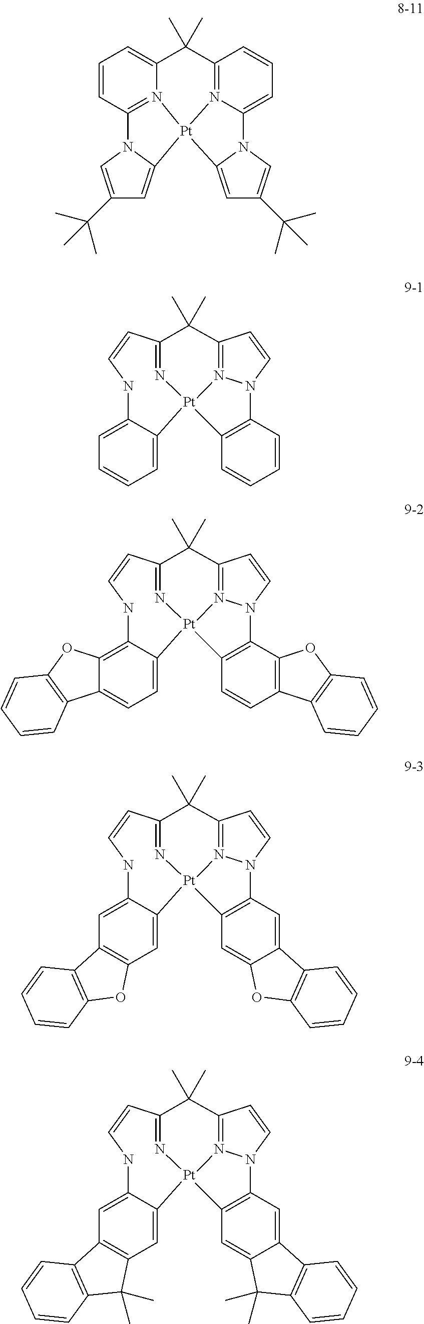

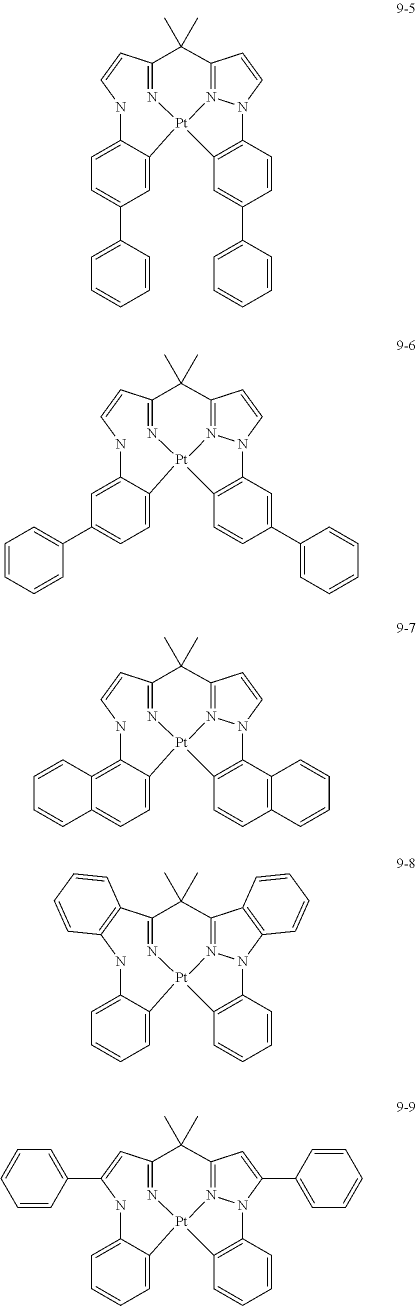

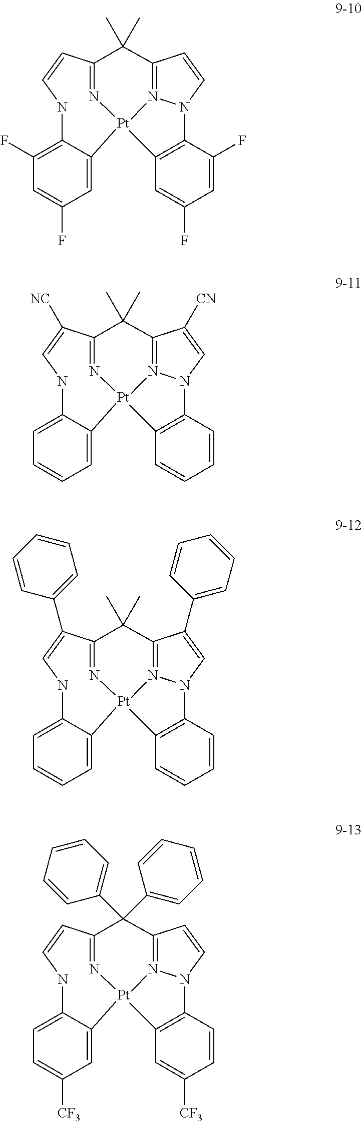

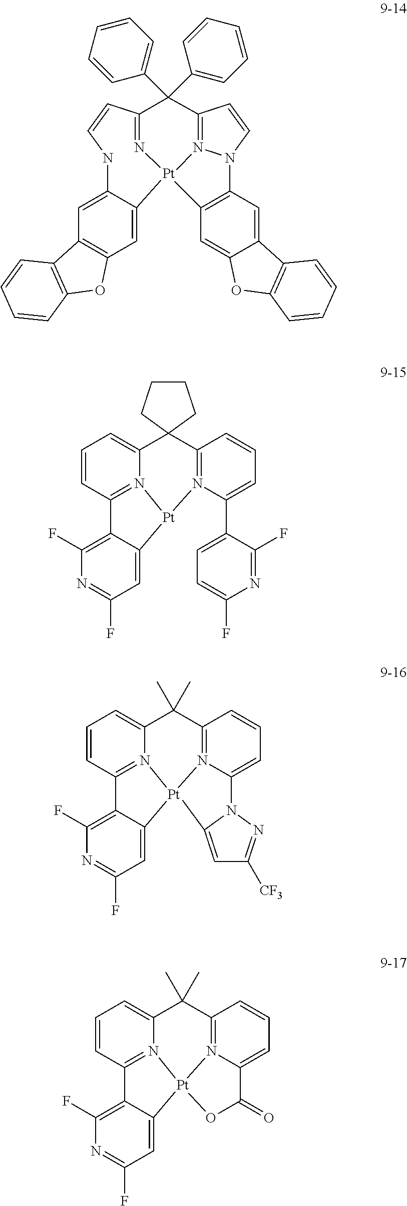

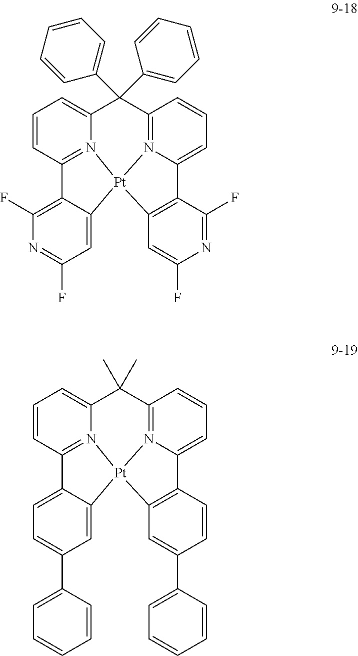

The platinum complex represented by formula (C-1) can be synthesized according to a variety of methods, for example, the method described in G. R. Newkome et al., Journal of Organic Chemistry, 53, 786 (1988), page 789, left column, line 53 to right column, line 7, the method described in page 790, left column, line 18 to line 38, the method described in page 790, right column, line 19 to line 30, and combinations of these methods, and the method described in H. Lexy et al., Chemische Berichte, 113, 2749 (1980), page 2752, line 26 to line 35 can be used.

For example, the platinum complex represented by formula (C-1) can be obtained at room temperature or lower or by heating a ligand or the dissociation product thereof and a metal compound (other than ordinary heating, a method of heating with a microwave is also effective) in the presence of a solvent (e.g., a halogen solvent, an alcohol solvent, an ether solvent, an ester solvent, a ketone solvent, a nitrile solvent, an amide solvent, a sulfone solvent, a sulfoxide solvent, and water can be exemplified) or in the absence of a solvent, in the presence of a base (various organic and inorganic bases, e.g., sodium methoxide, potassium t-butoxide, triethylamine, potassium carbonate can be exemplified) or in the absence of a base.

In the invention, when the compound represented by formula (C-1) is contained in a light-emitting layer, the content of the compound is preferably 1% by mass to 30% by mass in the light-emitting layer, more preferably 3% by mass to 25% by mass, and still more preferably 5% by mass to 20% by mass.

In the invention, besides the complex compounds, an iridium (Ir) complex can be used in combination as a light-emitting material. As the iridium (Ir) complex to be used in combination, a compound represented by the following formula (PQ-1) is preferably used.

The compound represented by formula (PQ-1) is described below.

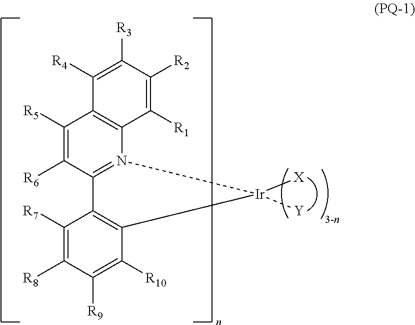

In formula (PQ-1), each of R₁ to R₁₀ represents a hydrogen atom or a substituent. If possible, the substituents may be bonded to each other to form a ring. X—Y represents a bidentate monoanionic ligand. n represents an integer of 1 to 3.

As the substituents represented by R₁ to R₁₀, above substituent group A can be exemplified. Each of R₁ to R₁₀ preferably represents a hydrogen atom, an alkyl group, an aryl group, an amino group, an alkoxy group, an aryloxy group, a heterocyclic oxy group, a cyano group, a heterocyclic group, a silyl group, a silyloxy group, or a fluoro group, more preferably a hydrogen atom, an alkyl group, an aryl group, an amino group, an alkoxy group, a cyano group, a silyl group, or a fluoro group, still more preferably a hydrogen atom, an alkyl group or an aryl group, still yet preferably a hydrogen atom, a methyl group, an ethyl group, an isopropyl group, a t-butyl group, a neopentyl group, an isobutyl group, a phenyl group, a naphthyl group, a phenanthryl group, or a tolyl group, yet further preferably a hydrogen atom, a methyl group or a phenyl group. If possible, substituents may be bonded to each other to form a ring.

n is preferably 2 or 3, and more preferably 2.

X—Y represents a bidentate monoanionic ligand. These ligands do not directly contribute to light emission characteristics but are considered to be capable of controlling light emission characteristics of the molecules. It is possible that “3−n” is 0, 1 or 2. Bidentate monoanionic ligands for use in light-emitting materials can be selected from among those well-known in the industry. As bidentate monoanionic ligands, the ligands described in Lamansky et al., PCT Application WO 02/15645, pages 89 to 90 can be exemplified, but the invention is not restricted thereto. Preferred bidentate monoanionic ligands include acetylacetonate (acac), picolinate (pic), and derivatives thereof. In view of stability of the complex and high yield of light emitting quantum, the bidentate monoanionic ligand is preferably acetylacetonate in the invention.

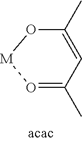

In the structural formula of the above acetylacetonate, M represents a metal atom to coordinate.

The compound represented by formula (PQ-1) is preferably a compound represented by the following formula (PQ-2).

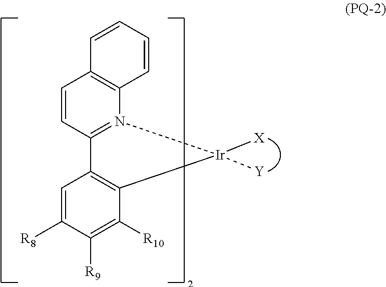

In formula (PQ-2), each of R₈ to R₁₀ represents a hydrogen atom or a substituent. If possible, the substituents may be bonded to each other to form a ring. X—Y represents a bidentate monoanionic ligand.

R₈ to R₁₀ and X—Y have the same meaning with R₈ to R₁₀ and X—Y in formula (PQ-1) respectively and preferred ranges are also the same.

The compound represented by formula (PQ-1) is preferably a compound represented by the following formula (PQ-3).

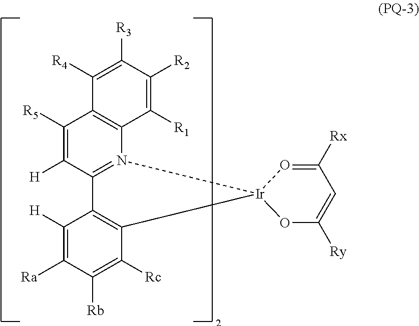

In formula (PQ-3), R₁ to R₅ have the same meaning as in formula (PQ-1). Each of Ra, Rb and Rc independently represents a hydrogen atom or an alkyl group, provided that one of Ra, Rb and Rc represents a hydrogen atom and other two represent an alkyl group. Each of Rx and Ry independently represents an alkyl group or a phenyl group.

Formula (PQ-3) will be described.

R₁ to R₅ have the same meaning as in formula (PQ-1), and each of R₁ to R₅ preferably represents a hydrogen atom, an alkyl group, an aryl group, a fluoro group or a cyano group, and more preferably a hydrogen atom, an alkyl group having 1 to 5 carbon atoms, a phenyl group, a fluoro group, or a cyano group. If possible, each of the substituents may have a substituent, and as the substituents, the groups in the following substituent group Z can be exemplified.

### (Substituent Group Z)

An alkyl group having 1 to 6 carbon atoms, an alkenyl group having 2 to 6 carbon atoms, a phenyl group, an aromatic heterocyclic group having 5 to 10 carbon atoms, an alkoxy group having 1 to 4 carbon atoms, a phenoxy group, a fluoro group, a silyl group, an amino group, a cyano group, and a group obtained by combining these groups.

When each of R₁ to R₅ has a plurality of substituents, these substituents may be linked to each other to form an aromatic hydrocarbon ring.

Each of R₁ to R₅ preferably represents a hydrogen atom, a methyl group, an ethyl group, an isobutyl group, a t-butyl group, a fluoro group, a phenyl group, a cyano group, or a trifluoromethyl group, more preferably a hydrogen atom, a methyl group, an isobutyl group, a fluoro group, a phenyl group, or a cyano group, still more preferably a hydrogen atom, a methyl group, an isobutyl group, or a phenyl group, still yet preferably a hydrogen atom, a methyl group, or an isobutyl group, and especially preferably a hydrogen atom.

Each of Ra, Rb and Rc independently represents a hydrogen atom or an alkyl group (preferably an alkyl group having 1 to 5 carbon atoms), provided that at least one of Ra, Rb and Rc represents a hydrogen atom, preferably Rb or Rc represents a hydrogen atom, and more preferably Rb represents a hydrogen atom.

When each of Ra, Rb and Rc represents a group other than a hydrogen atom, the group is preferably a methyl group, an ethyl group, a propyl group, an isopropyl group, an n-butyl group, an isobutyl group, a t-butyl group, an n-pentyl group, an isoamyl group, a t-amyl group, or an n-hexyl group, more preferably a methyl group, an ethyl group, a propyl group, an isopropyl group, or a t-butyl group, still more preferably a methyl group or an ethyl group, and especially preferably a methyl group.

Each of Rx and Ry independently represents an alkyl group or a phenyl group. The alkyl group is preferably an alkyl group having 1 to 5 carbon atoms.

Each of Rx and Ry preferably represents a methyl group, a t-butyl group or a phenyl group, and more preferably a methyl group.

The specific examples of the compound represented by formula (PQ-1) are enumerated below, but the invention is not restricted thereto.

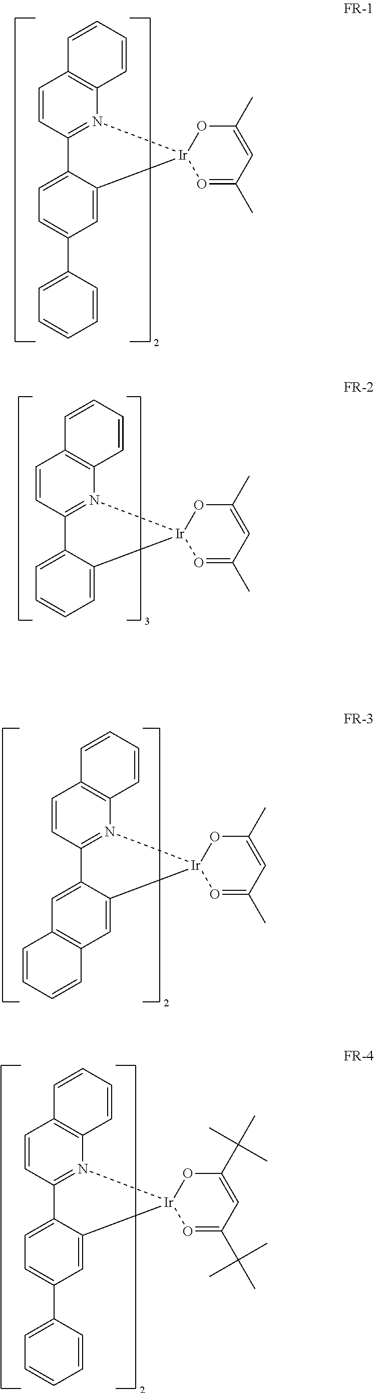

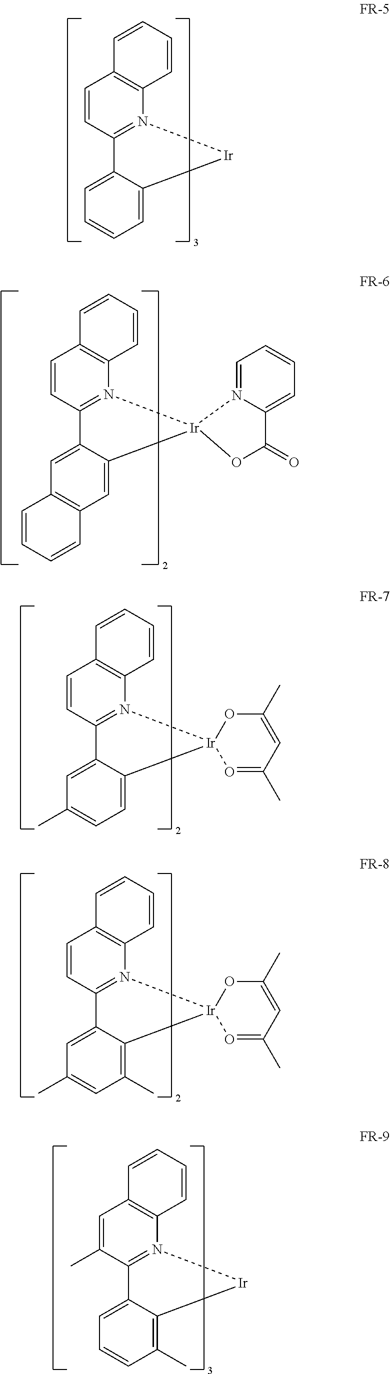

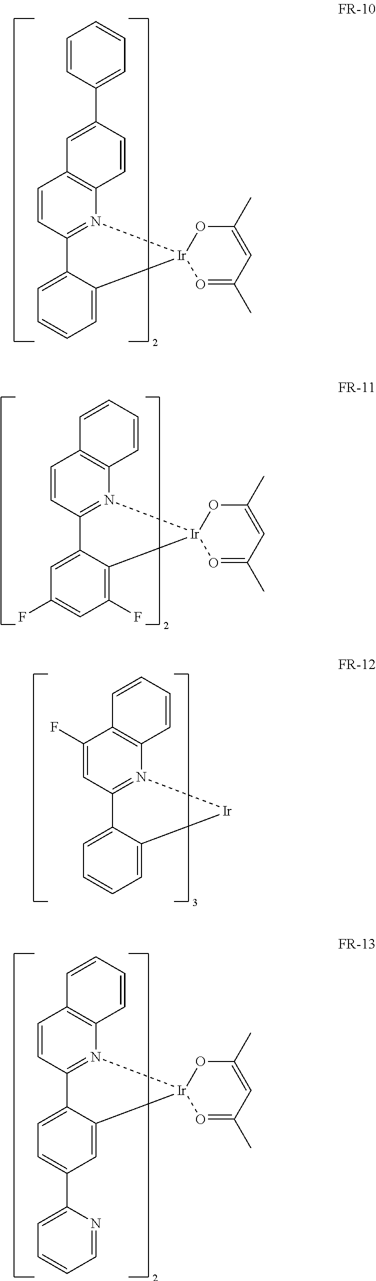

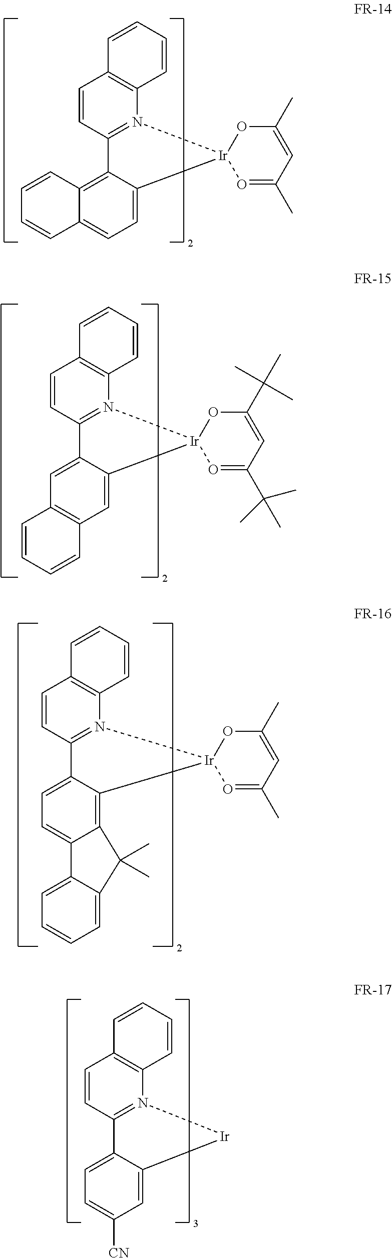

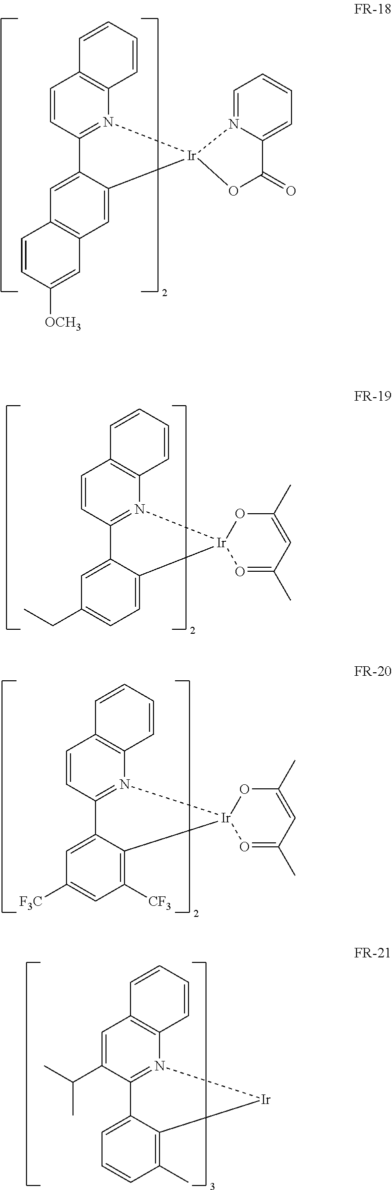

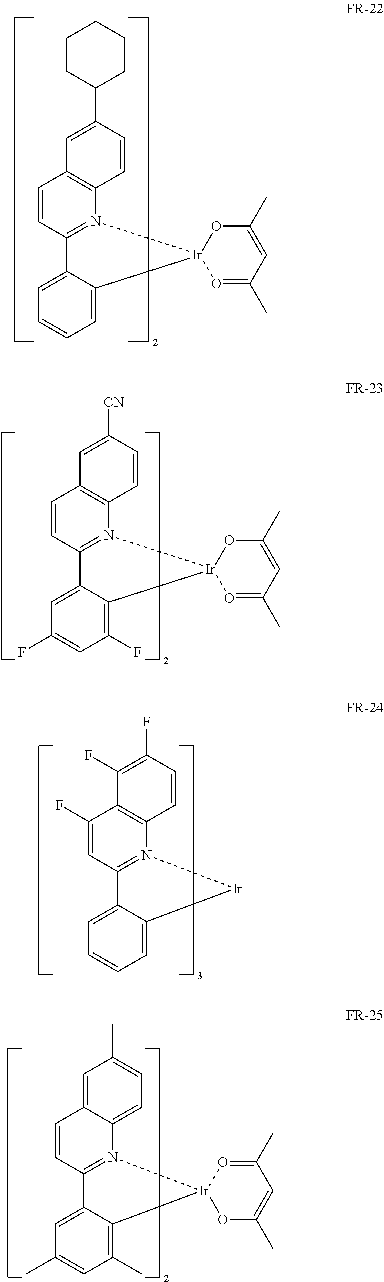

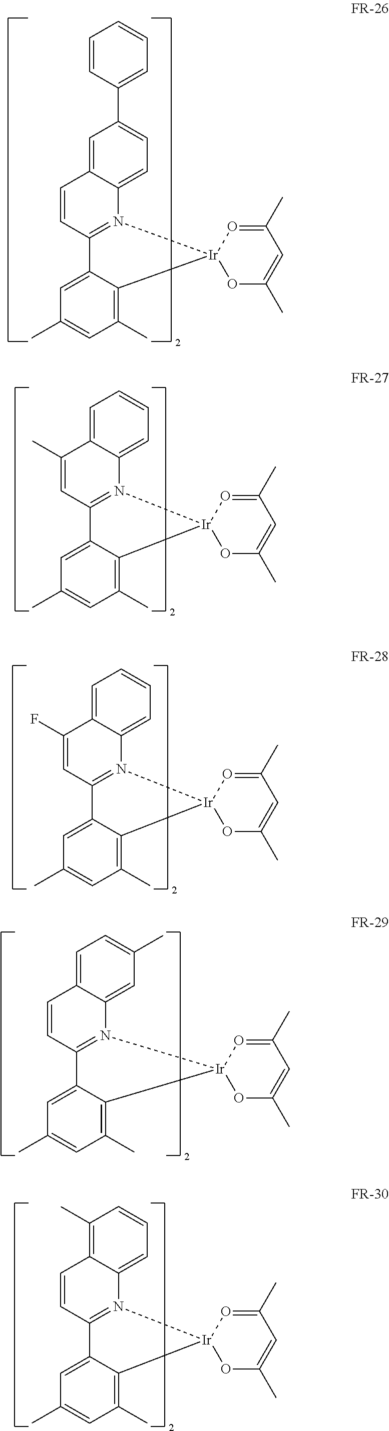

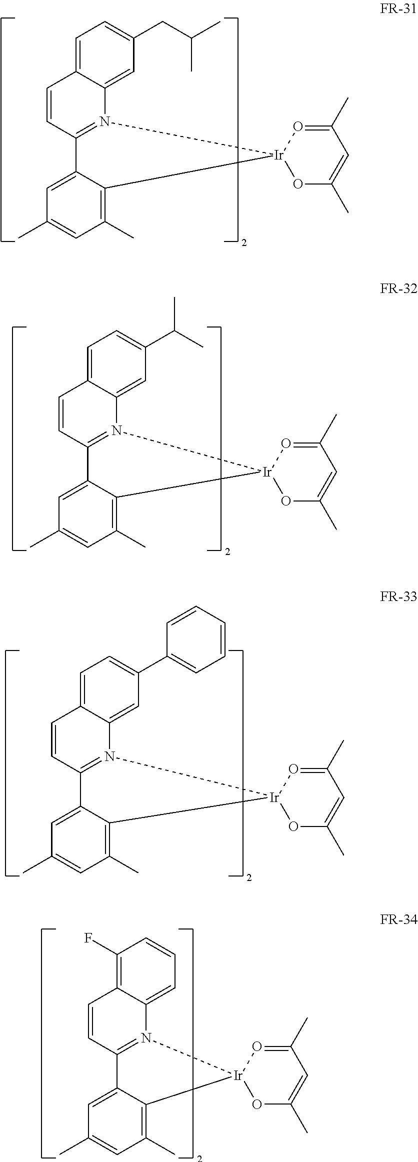

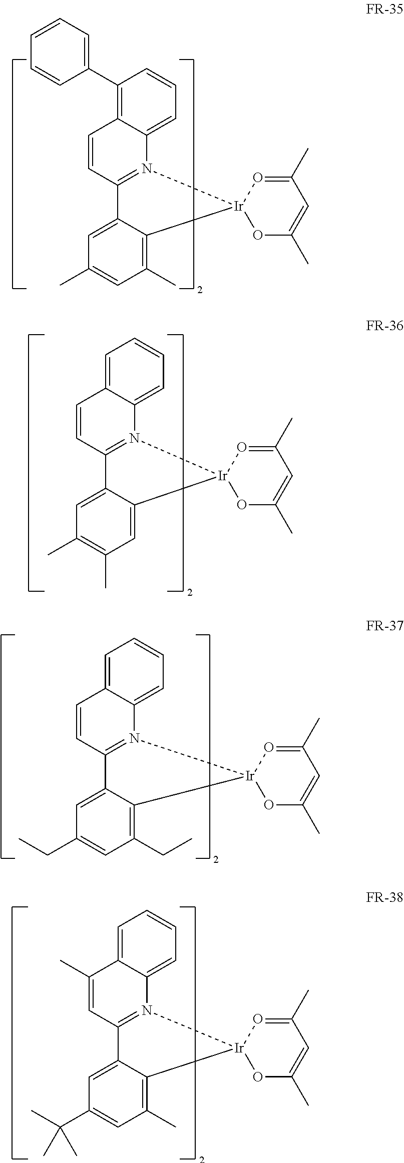

The compounds exemplified as the compounds represented by formula (PQ-1) can be synthesized according to various methods, such as the methods described in Patent 3929632. For example, FR-2 can be synthesized according to the method described in Patent 3929632, page 18, lines 2 to 13, with 2-phenylquinoline as the starting material. Further, FR-3 can be synthesized according to the method described in Patent 3929632, page 18, line 14 to page 19, line 8, with 2-(2-naphthyl)quinoline as the starting material.

In the invention, when the compound represented by formula (PQ-1) is contained in a light-emitting layer, the content is preferably 0.1% by mass to 30% by mass in the light-emitting layer, more preferably 2% by mass to 20% by mass, and more preferably 5% by mass to 15% by mass.

A light-emitting material in a light-emitting layer is contained in an amount of generally 0.1% by mass to 50% by mass to the content of all the compounds forming the light-emitting layer, preferably 1% by mass to 50% by mass from the point of durability and external quantum efficiency, and more preferably 2% by mass to 40% by mass.

The thickness of a light-emitting layer is not especially restricted, but is generally preferably 2 nm to 500 nm, more preferably 3 nm to 200 nm from the viewpoint of external quantum efficiency, and still more preferably 5 nm to 100 nm.

The light-emitting layer in the device of the invention may be formed of a light-emitting material alone, or may be constituted as a mixed layer of a host material and a light-emitting material. The kind of the light-emitting material may be one or two or more. The host material is preferably a charge-transporting material. The kind of the host material may be one or two or more. For example, a composition obtained by mixing an electron-transporting host material and a hole-transporting host material can be exemplified. Further, a material not having a charge-transporting property and not emitting light may be contained in a light-emitting layer.

Further, a light-emitting layer may be a monolayer or may be a multilayer of two or more layers. The same light-emitting material and host material may be contained in each layer, or every layer may contain different materials. When a light-emitting layer consists of two or more layers, each layer may emit light in different luminescent colors.

### <Host Material>

The host material is a compound primarily performing injection and transportation of charge in a light-emitting layer, which is a compound that does not substantially emit light. In the specification of the invention, the terms “does not substantially emit light” means that the quantity of light emission from the compound that does not substantially emit light is preferably 5% or less of the total quantity of light emission of the device as a whole, more preferably 3% or less, and still more preferably 1% or less.

The compound represented by formula (1) of the invention can be used as the host material. In this case, the compound is preferably used in combination with the platinum complex represented by formula (C-1). When they are used in combination, the mass ratio of the compound represented by formula (1) and the platinum complex represented by formula (C-1) is preferably 99/1 to 3/1, and more preferably 95/1 to 5/1.

In addition to the above, as host materials that can be used in the invention, the following compounds can be exemplified.

Condensed ring hydrocarbon compounds (naphthalene, anthracene, phenanthrene, triphenylene, and pyrene), pyrrole, indole, carbazole, azaindole, azacarbazole, triazole, oxazole, oxadiazole, pyrazole, imidazole, thiophene, polyarylalkane, pyrazoline, pyrazolone, phenylenediamine, arylamine, amino-substituted chalcone, styrylanthracene, fluorenone, hydrazone, stilbene, silazane, aromatic tertiary amine compounds, styrylamine compounds, porphyrin compounds, polysilane compounds, electrically conductive polymeric oligomers such as poly(N-vinylcarbazole), aniline copolymers, thiophene oligomers, and polythiophene, organic silane, carbon films, pyridine, pyrimidine, triazine, imidazole, pyrazole, triazole, oxazole, oxadiazole, fluorenone, anthraquinodimethane, anthrone, diphenylquinone, thiopyran dioxide, carbodiimide, fluorenylidenemethane, distyrylpyrazine, fluorine-substituted aromatic compounds, heterocyclic tetracarboxylic anhydrides such as naphthaleneperylene, phthalocyanine, metal complexes of 8-quinolinol derivatives, various metal complexes represented by metal complexes having metalphthalocyanine, benzoxazole or benzothiazole as a ligand, and derivatives thereof (which may have substitution products and condensed rings) can be exemplified.

Host materials which can be used in combination in the invention may be hole-transporting host materials or may be electron-transporting host materials, but hole-transporting host materials can be used.

In the invention, it is preferred for the light-emitting layer to contain a host material. The host material is preferably a compound represented by the following formula (4-1) or (4-2).

In the invention, it is more preferred that the light-emitting layer contains at least one or more compounds represented by formula (4-1) or (4-2).

When the compound represented by formula (4-1) or (4-2) is contained in the light-emitting layer in the invention, the content of the compound represented by formula (4-1) or (4-2) is preferably 30% by mass to 100% by mass in the light emitting layer, more preferably 40% by mass to 100% by mass, and especially preferably 50% by mass to 100% by mass. When the compound represented by formula (4-1) or (4-2) is contained in two or more organic layers, the compound is preferably contained in the above range of the content in each layer.

The compound represented by formula (4-1) or (4-2) may be contained alone in any organic layer or a plurality of the compounds represented by formula (4-1) or (4-2) may be contained in combination in an arbitrary ratio.

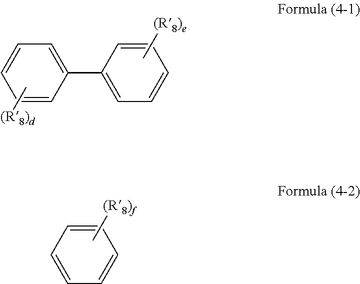

In formulae (4-1) and (4-2), each of d and e represents an integer of 0 to 3, and at least either one is 1 or more. f represents an integer of 1 to 4. R′₈ represents a substituent, and each of d, e and f represents 2 or more, two or more R′₈ may be different from or the same with each other. At least one R′₈ represents a carbazole group represented by the following formula (5).

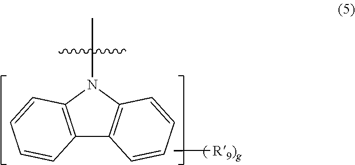

In formula (5), each R′₉ independently represents a substituent. g represents an integer of 0 to 8.

Each R′₈ independently represents a substituent. The specific examples of the substituents include a halogen atom, an alkoxy group, a cyano group, a nitro group, an alkyl group, an aryl group, a heterocyclic group, and the substituent represented by formula (5). When R′₈ does not represent formula (5), the substituent is preferably an alkyl group having 10 or less carbon atoms or a substituted or unsubstituted aryl group having 10 or less carbon atoms, and more preferably an alkyl group having 6 or less carbon atoms.

Each R′₉ independently represents a substituent. The specific examples of the substituents include a halogen atom, an alkoxy group, a cyano group, a nitro group, an alkyl group, an aryl group, and a heterocyclic group, preferably an alkyl group having 10 or less carbon atoms, and a substituted or unsubstituted aryl group having 10 or less carbon atoms, and more preferably an alkyl group having 6 or less carbon atoms.

g represents an integer of 0 to 8. From the viewpoint of not excessively shielding the carbazole structure for performing transportation of charge, g is preferably 0 to 4. Further, from the aspect of easiness of synthesis, when the carbazole has a substituent, those having substituents symmetrically to the nitrogen atom are preferred.

In formula (4-1), the sum of d and e is preferably 2 or more in the point of retaining charge transportation performance. It is preferred that R′_{g} is substituted at meta-position to the other benzene ring. This is for the reason that steric hindrance of contiguous substituents is great in substitution at ortho-position and cleavage of bonding is liable to occur, so that durability lowers. Further, in substitution at para-position, the molecular shape approaches to a stiff rod state and crystallization is liable to occur, as a result device easily deteriorates under high temperature conditions. Specifically, a compound represented by the following structure is preferred.

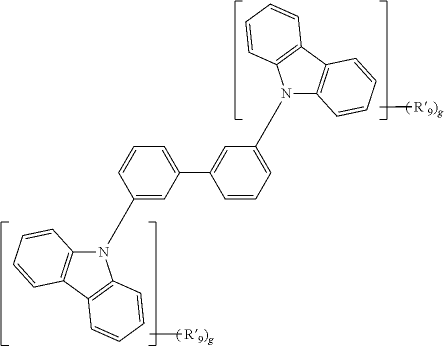

In formula (4-2), f is preferably 2 or more in view of retaining charge transportation performance. When f is 2 or 3, R′₈ preferably substitute at meta-position to each other from the same viewpoint. Specifically, a compound represented by the following structure is preferred.

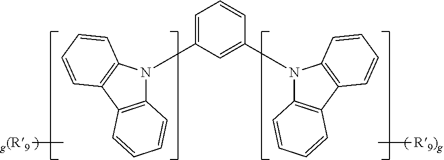

When formulae (4-1) and (4-2) have hydrogen atoms, isotopes of hydrogen atoms (deuterium atoms) are also included in the hydrogen atoms. In such a case, all the hydrogen atoms in the compounds may be substituted with hydrogen isotopes, or the compounds may be mixtures partially containing hydrogen isotopes. Preferred are compounds in which R′₉ in formula (5) is substituted with a deuterium atom, and especially preferably the following structures are exemplified.

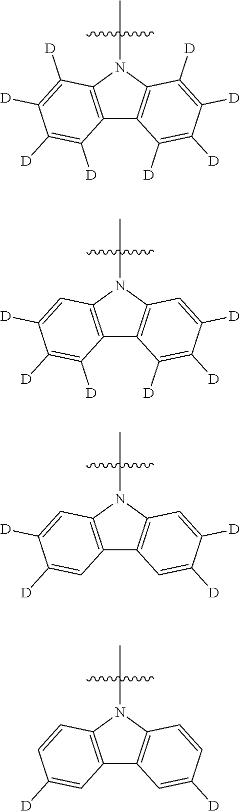

The atoms further constituting substituents also include the isotopes thereof.

The compounds represented by formulae (4-1) and (4-2) can be synthesized by combining various known synthesis methods. Most generally, concerning the carbazole compounds, synthesis by dehydrogenation aromatization after Aza-Cope arrangement of the condensation product of aryl hydrazine and cyclohexane derivative (L. F. Tieze and Th. Eicher, translated by Takano and Ogasawara, Precision Organic Syntheses, p. 339, published by Nanko-Do) is exemplified. Further, concerning the coupling reaction of the obtained carbazole compound and aryl halide compound using a palladium catalyst, the methods described in Tetrahedron Letters, Vol. 39, p. 617 (1998), ibid., Vol. 39, p. 2367 (1998), and ibid., Vol. 40, p. 6393 (1999) are exemplified. The reaction temperature and reaction time are not especially restricted and the conditions in the above documents are applied. Concerning some compounds, such as mCP, commercially available products can be preferably used.

It is preferred that the films of the compounds represented by formulae (4-1) and (4-2) are formed according to a vacuum deposition process, but a wet process such as solution coating can also be preferably used. The molecular weight of the compound is preferably 2,000 or less in view of deposition aptitude and solubility, more preferably 1,200 or less, and especially preferably 800 or less. In view of deposition aptitude, too small a molecular weight results in too small a vapor pressure, conversion from a gaseous phase to a solid phase does not occur and it becomes difficult to form an organic layer, so that the molecular weight is preferably 250 or more, and especially preferably 300 or more.

Compounds having the structures shown below, or compounds obtained by substituting one or more hydrogen atoms of the following compounds with deuterium atoms are preferably used as the compounds of formulae (4-1) and (4-2).

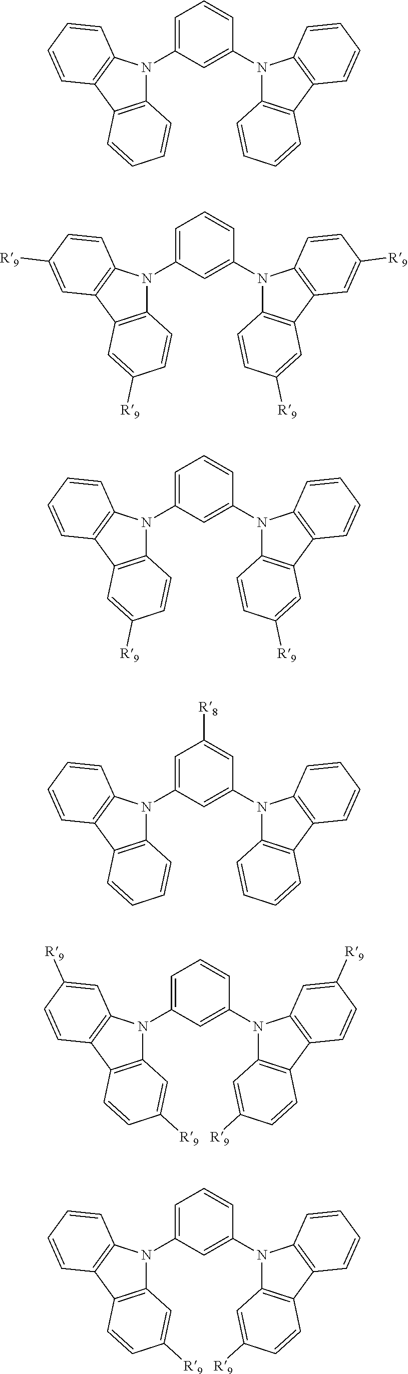

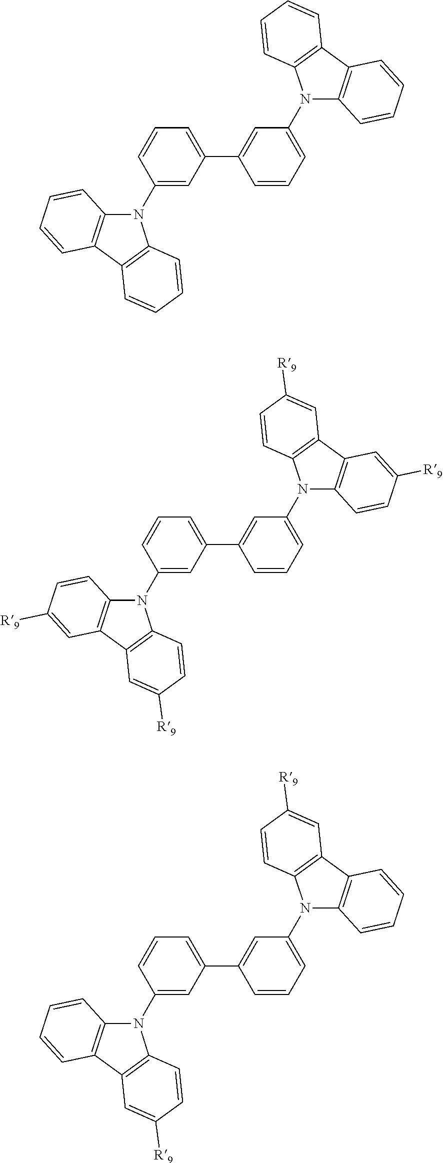

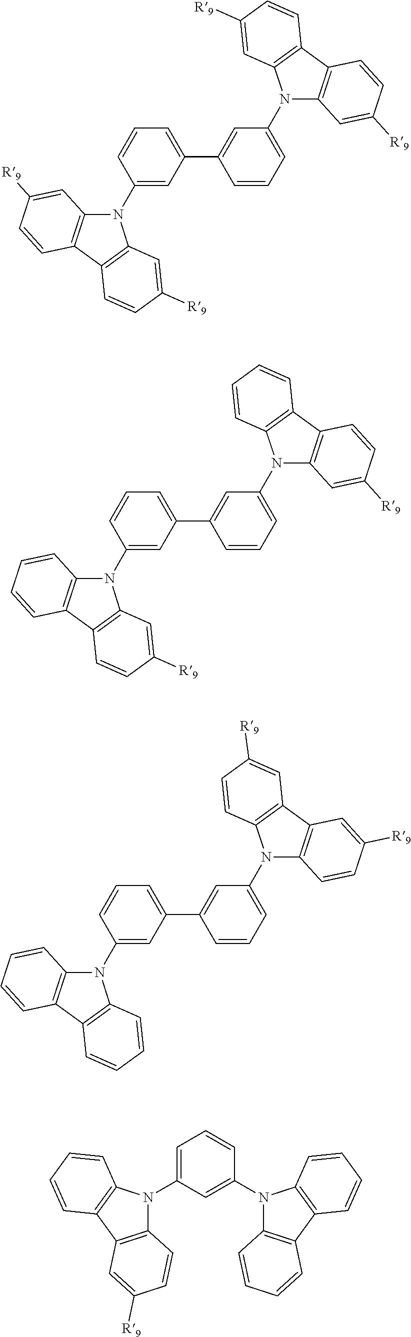

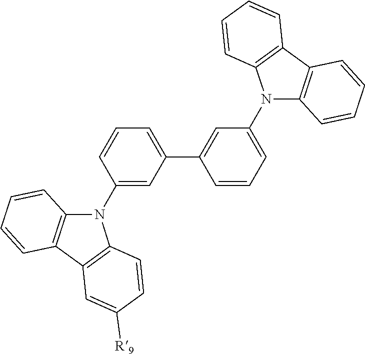

In the above specific examples, R′⁸ has the same meaning with R′⁸ in formula (4-1) or (4-2), and R′⁹ has the same meaning with R′⁹ in formula (5).

The specific examples of the compounds represented by formulae (4-1) and (4-2) of the invention are shown below, but the invention is not restricted thereto. In the following specific examples, D represents a deuterium.

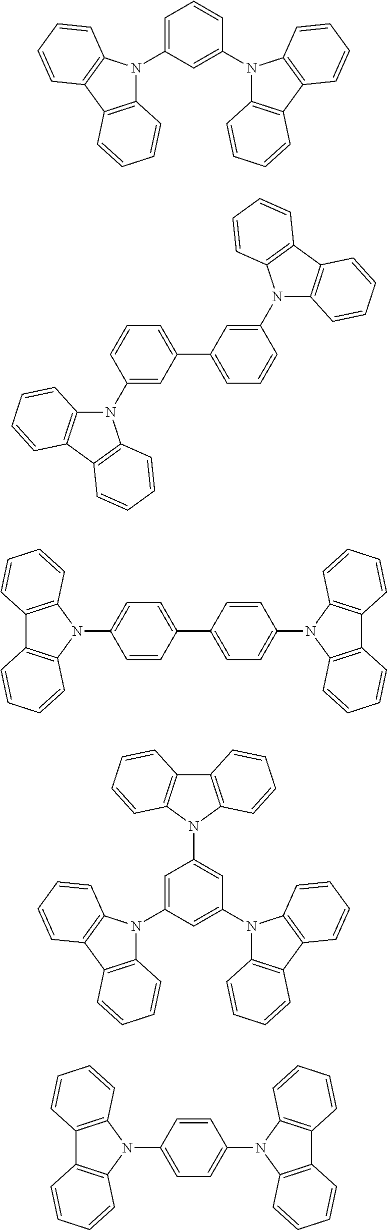

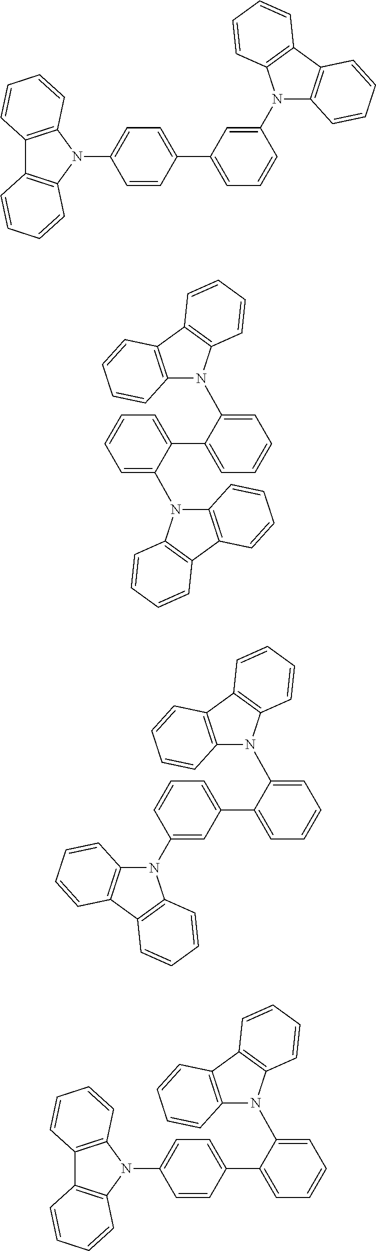

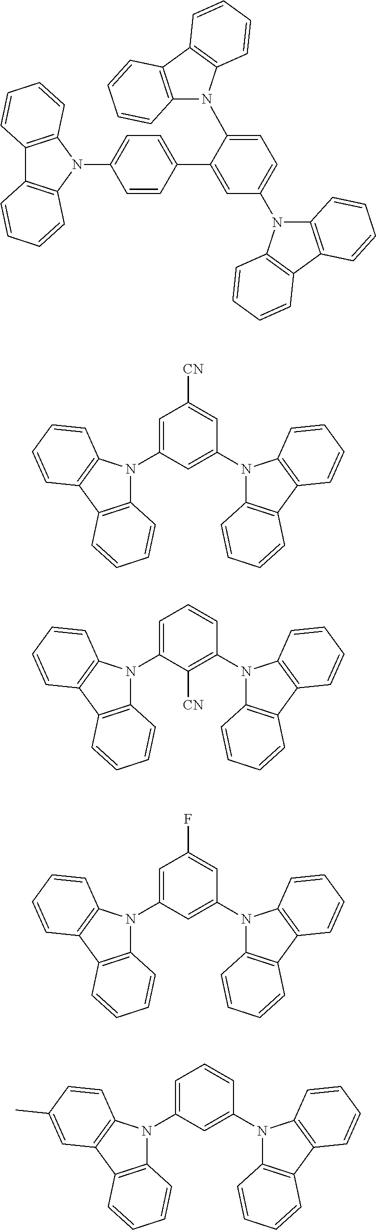

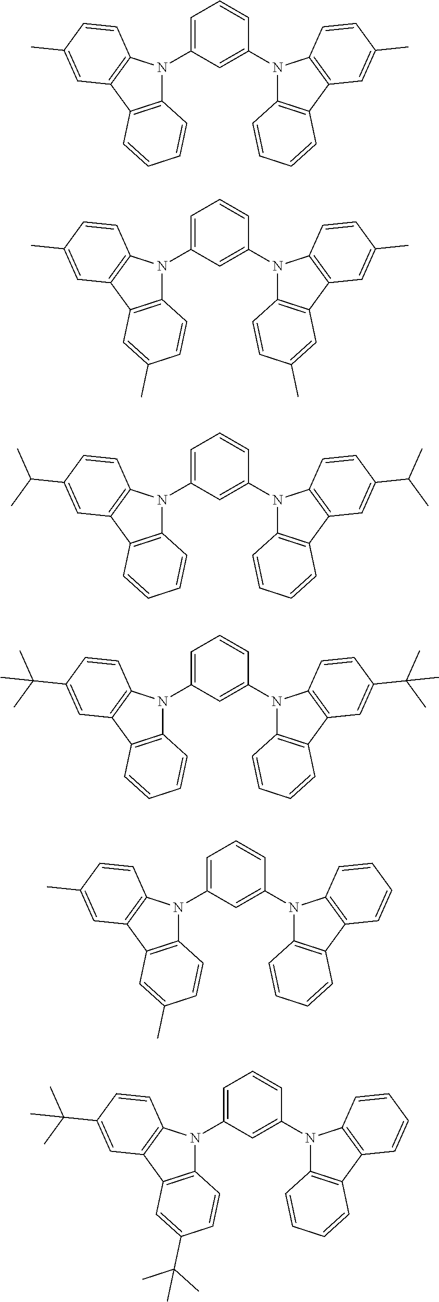

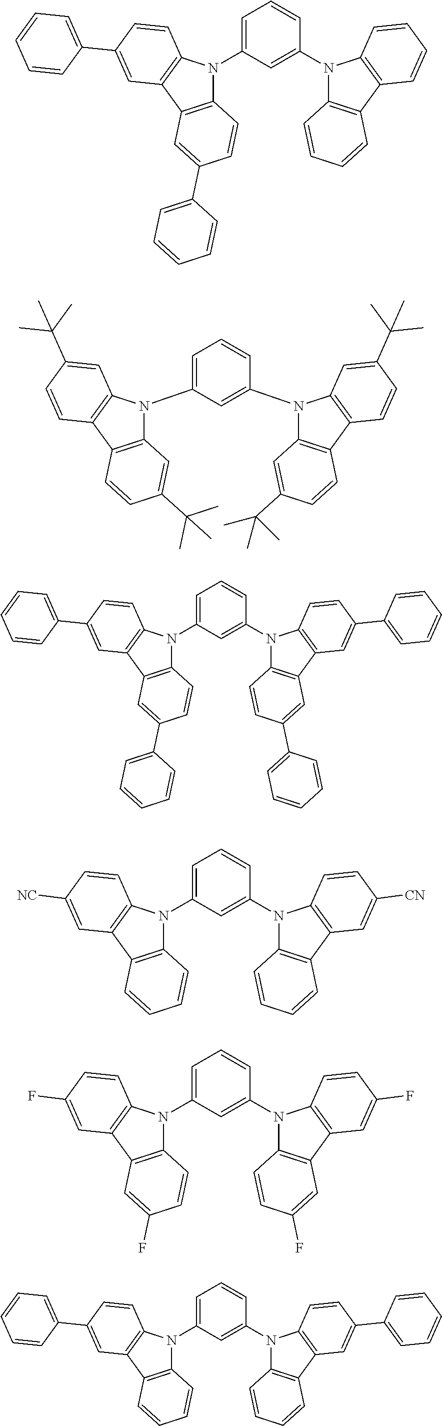

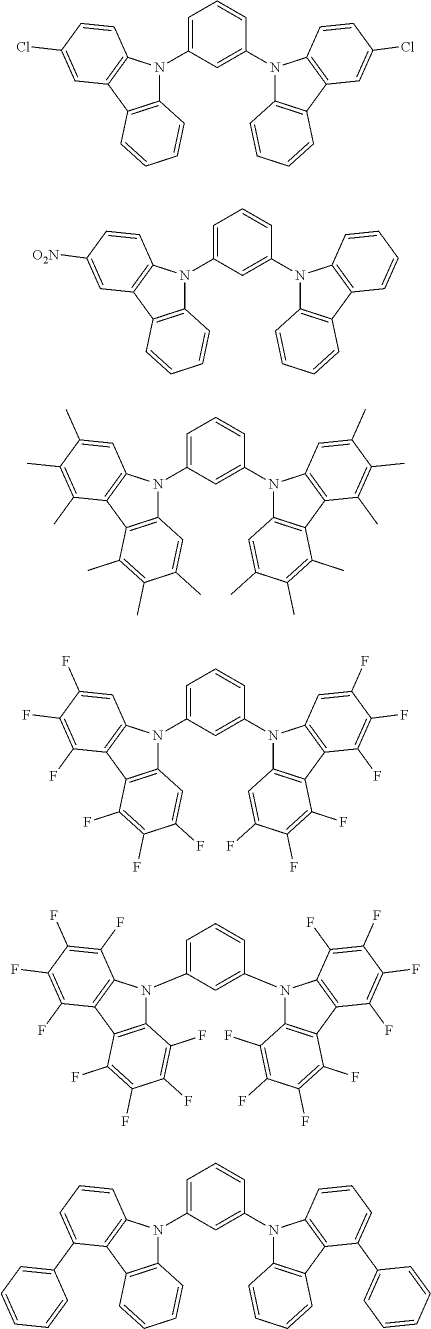

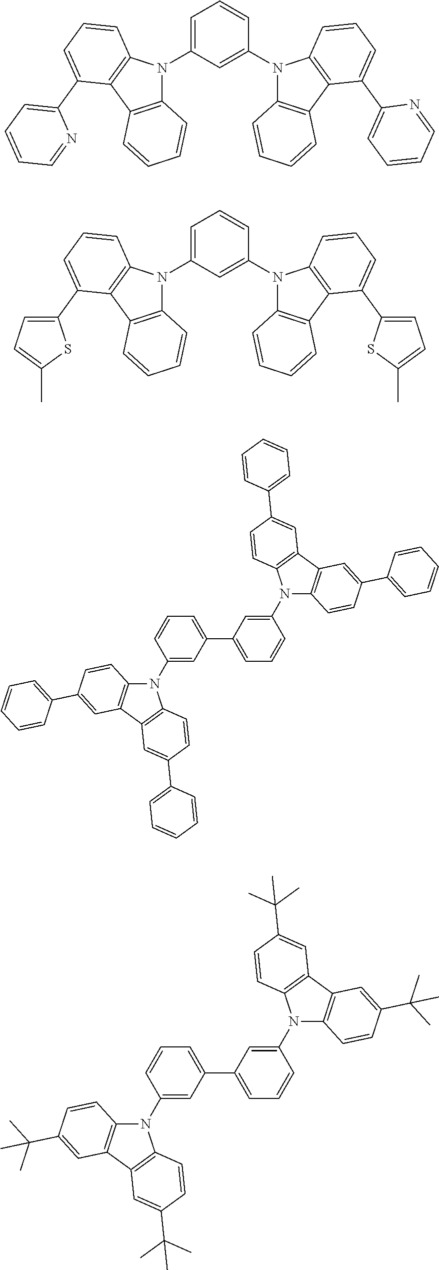

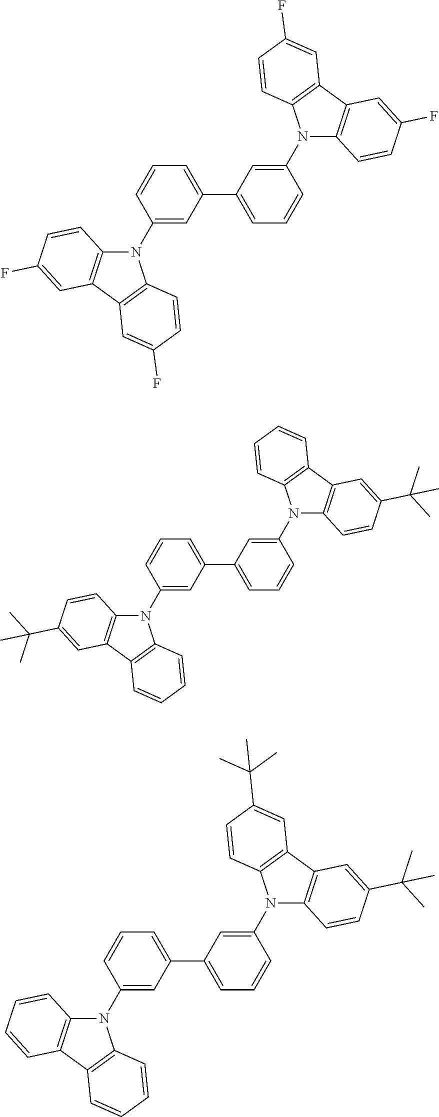

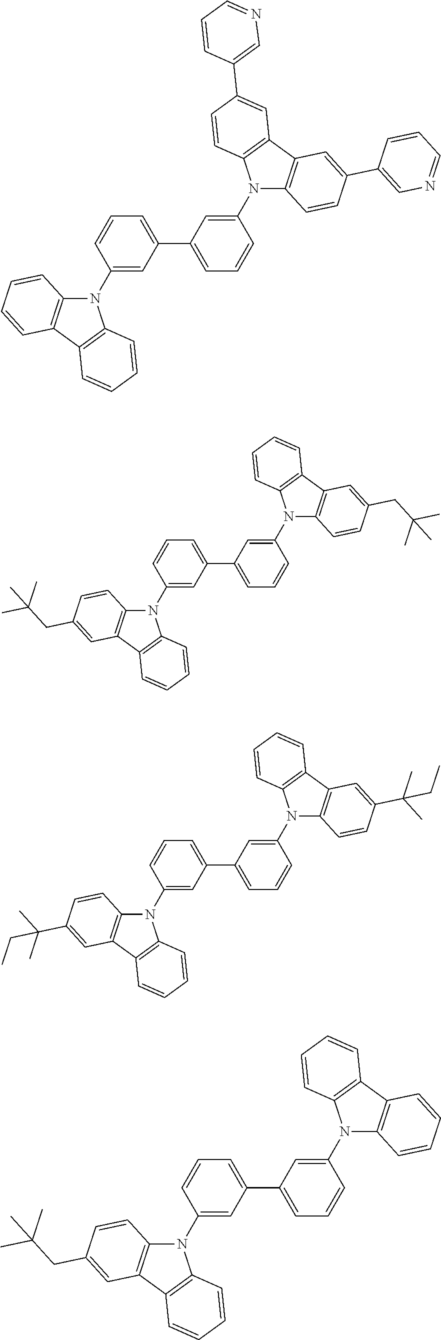

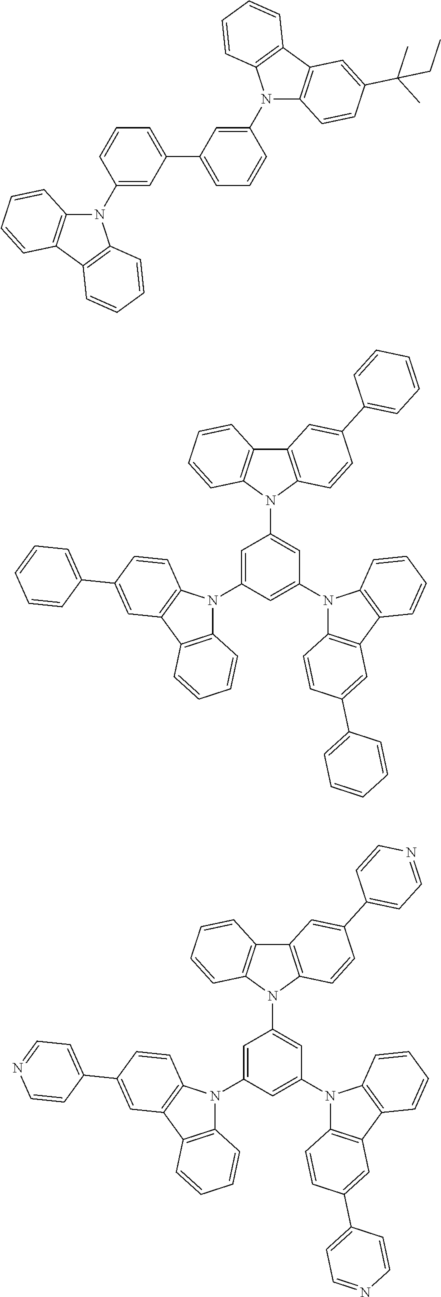

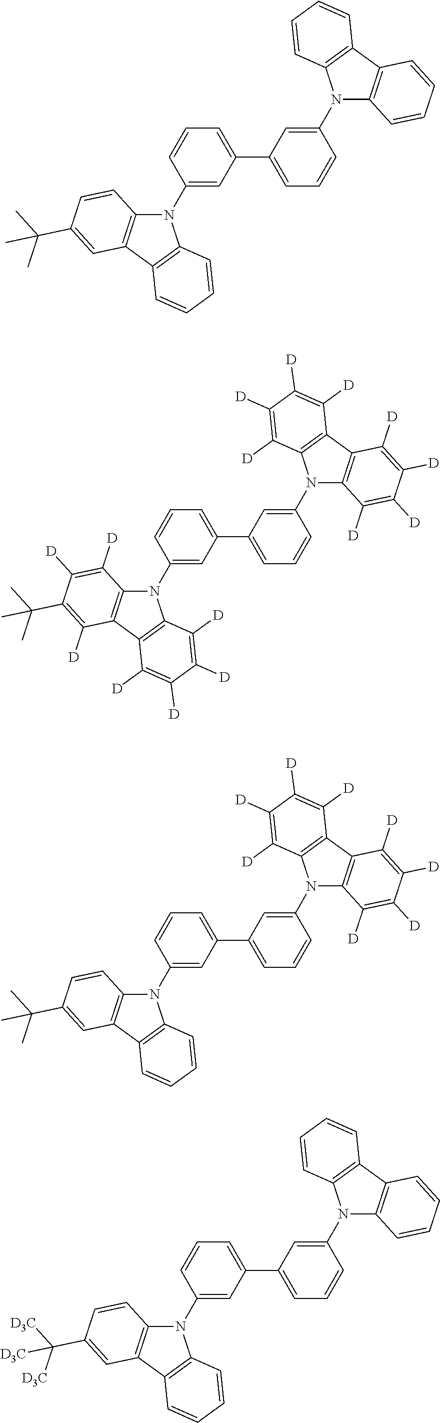

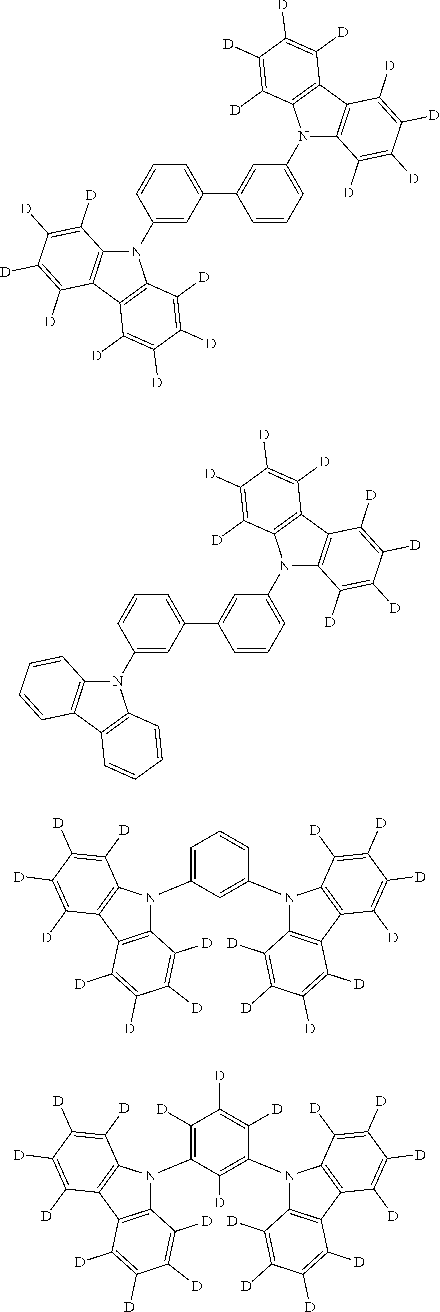

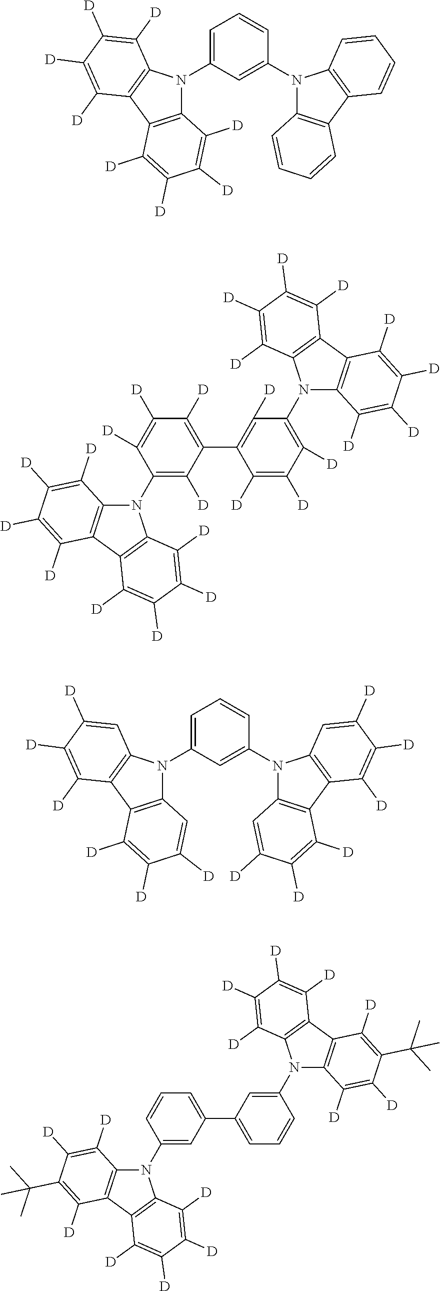

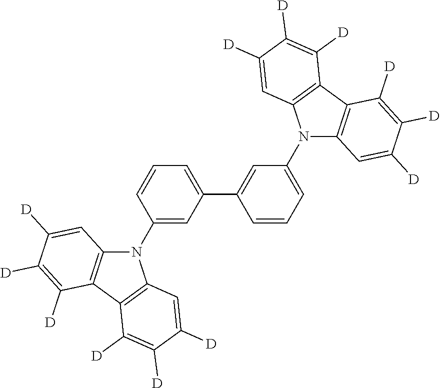

In the light-emitting layer of the invention, it is preferred that the triplet minimum excited state energy (T₁ energy) of the above each host material is higher than T₁ energy of the phosphorescent material in the points of chromaticity, light emission efficiency and driving durability.

The content of the host compound in the invention is not especially restricted, but the amount is preferably 15% by mass or more and 95% by mass or less to the gross mass of the compounds for forming the light-emitting layer from the points of light emission efficiency and driving durability. The content of the compound represented by formula (1) is preferably 50% by mass or more and 99% by mass or less in all the host materials.

In the organic electroluminescence device according to the invention, the electrodes include the anode, and a charge-transporting layer is provided between the light-emitting layer and the anode, and it is preferred for the charge-transporting layer to contain a carbazole compound.

### (Charge-Transporting Layer)

A charge-transporting layer means a layer in which charge transportation occurs at the time of voltage application to the organic electroluminescence device. Specifically, a hole-injecting layer, a hole-transporting layer, an electron-blocking layer, a light-emitting layer, a hole-blocking layer, an electron-transporting layer and an electron-injecting layer are exemplified. Preferred are a hole-injecting layer, a hole-transporting layer, an electron-blocking layer, and a light-emitting layer. A charge-transporting layer formed by a coating method is a hole-injecting layer, a hole-transporting layer, an electron-blocking layer, or a light-emitting layer, manufacture of an organic electroluminescence device by low cost and high efficiency becomes possible. As the charge-transporting layer, a hole-injecting layer, a hole-transporting layer, or an electron-blocking layer are more preferred.

### —Hole-Injecting Layer, Hole-Transporting Layer—

The hole-injecting layer and the hole-transporting layer are layers having functions of receiving holes from the anode or anode side and transporting the holes to the cathode side.

Concerning the hole-injecting layer and the hole-transporting layer, the items described in JP-A-2008-270736, paragraphs [0165] to [0167] can be applied to the invention.

It is preferred for the hole-injecting layer and the hole-transporting layer to contain a carbazole compound.

In the invention, the carbazole compound is preferably a carbazole compound represented by the following formula (a).

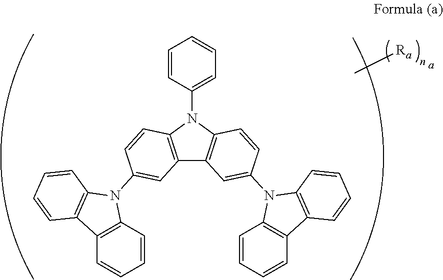

In formula (a), Rₐ represents a substituent capable of substituting on the hydrogen atom of the above structure, and when two or more Rₐ are present, these Rₐ may be the same with or different from each other. nₐ represents an integer of 0 to 8.

When the compound represented by formula (a) is used in a charge transporting layer, the content of the compound represented by formula (a) is preferably 50% by mass to 100% by mass, more preferably 80% by mass to 100% by mass, and especially preferably 95% by mass to 100% by mass.

Further, when the compound represented by formula (a) is used in a plurality of organic layers, the compound is preferably contained in the above range of the content in each layer.

The compound represented by formula (a) may be contained alone in any organic layer or a plurality of kinds of the compounds represented by formula (a) may be contained in combination in an arbitrary ratio.

In the invention, when the compound represented by formula (a) is contained in a hole-transporting layer, the thickness of the hole-transporting layer containing the compound represented by formula (a) is preferably 1 nm to 500 nm, more preferably 3 nm to 200 nm, and still more preferably 5 nm to 100 nm. The hole-transporting layer is preferably provided in contiguous to the light-emitting layer. The hole-transporting layer may have a monolayer structure containing one or two or more kinds of the above materials, or may have a multilayer structure comprising two or more layers of the same composition or different compositions.

As the substituents represented by Rₐ, a halogen atom, an alkoxy group, a cyano group, a nitro group, an alkyl group, an aryl group, and an aromatic heterocyclic group are specifically exemplified, preferably an alkyl group having 10 or less carbon atoms and a substituted or unsubstituted aryl group having 10 or less carbon atoms, and more preferably an alkyl group having 6 or less carbon atoms. t is preferably 0 to 4, and more preferably 0 to 2.

In the invention, the hydrogen atoms constituting formula (a) also contain isotopes of hydrogen atoms (deuterium atoms). In such a case, all the hydrogen atoms in the compounds may be substituted with hydrogen isotopes, or the compounds may be mixtures partially containing hydrogen isotopes.

The compounds represented by formulae (a) can be synthesized by combining various known synthesis methods. Most generally, concerning the carbazole compounds, synthesis by dehydrogenation aromatization after Aza-Cope arrangement of the condensation product of aryl hydrazine and cyclohexane derivative (L. F. Tieze and Th. Eicher, translated by Takano and Ogasawara, Precision Organic Syntheses, p. 339, published by Nanko-Do) is exemplified. Further, concerning the coupling reaction of the obtained carbazole compound and aryl halide compound using a palladium catalyst, the methods described in Tetrahedron Letters, Vol. 39, p. 617 (1998), ibid., Vol. 39, p. 2367 (1998), and ibid., Vol. 40, p. 6393 (1999) are exemplified. The reaction temperature and reaction time are not especially restricted and the conditions in the above documents are applied.

It is preferred that the films of the compounds represented by formulae (a) and (4-2) are formed according to a vacuum deposition process, but a wet process such as solution coating can also be preferably used. The molecular weight of the compound is preferably 2,000 or less in view of deposition aptitude and solubility, more preferably 1,200 or less, and especially preferably 800 or less. In view of deposition aptitude, too small a molecular weight results in too small a vapor pressure, conversion from a gaseous phase to a solid phase does not occur and it becomes difficult to form an organic layer, so that the molecular weight is preferably 250 or more, and especially preferably 300 or more.

The specific examples of the compounds represented by formula (a) of the invention are shown below, but the invention is not restricted thereto.

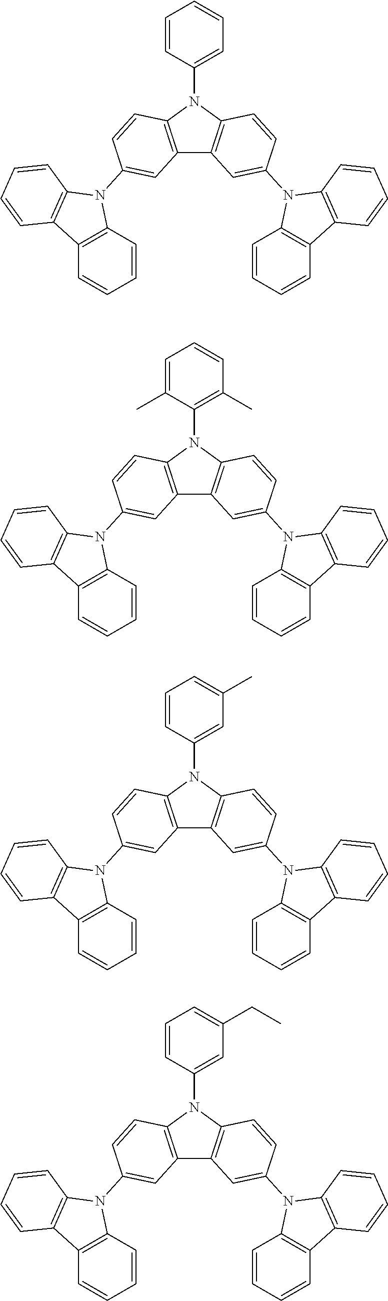

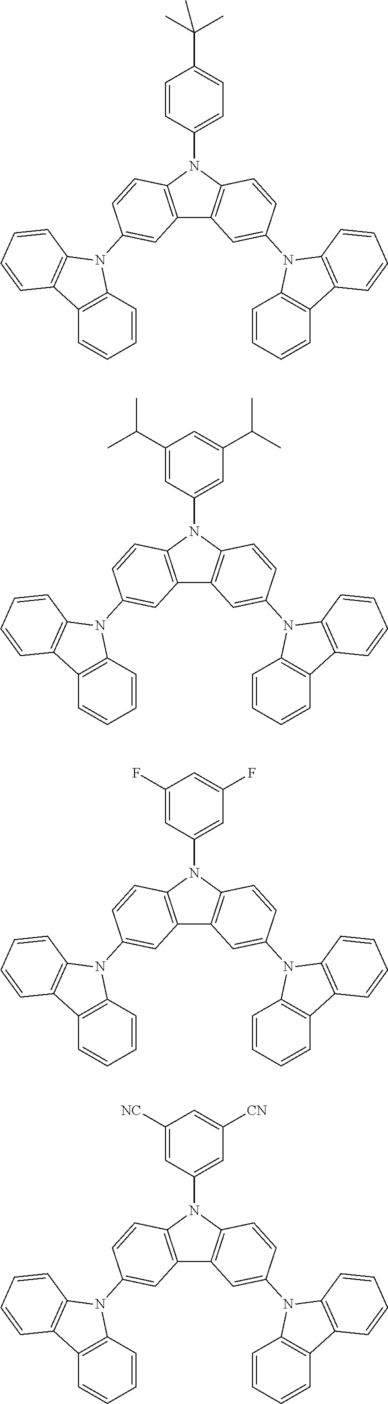

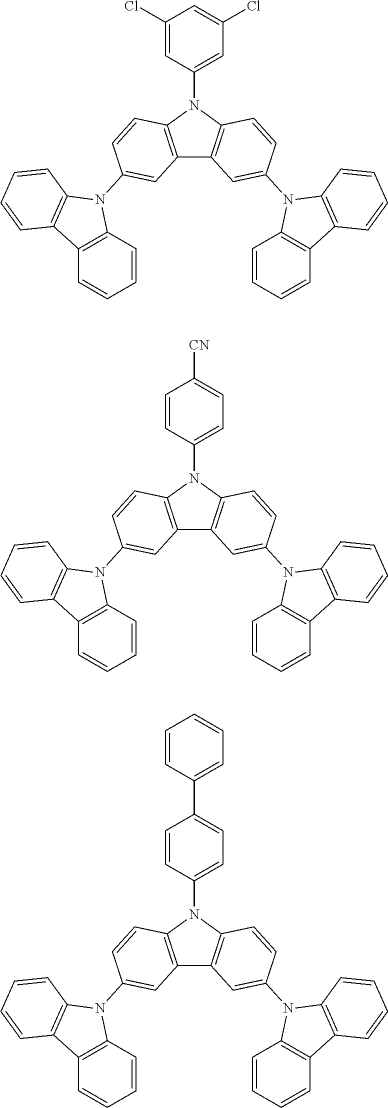

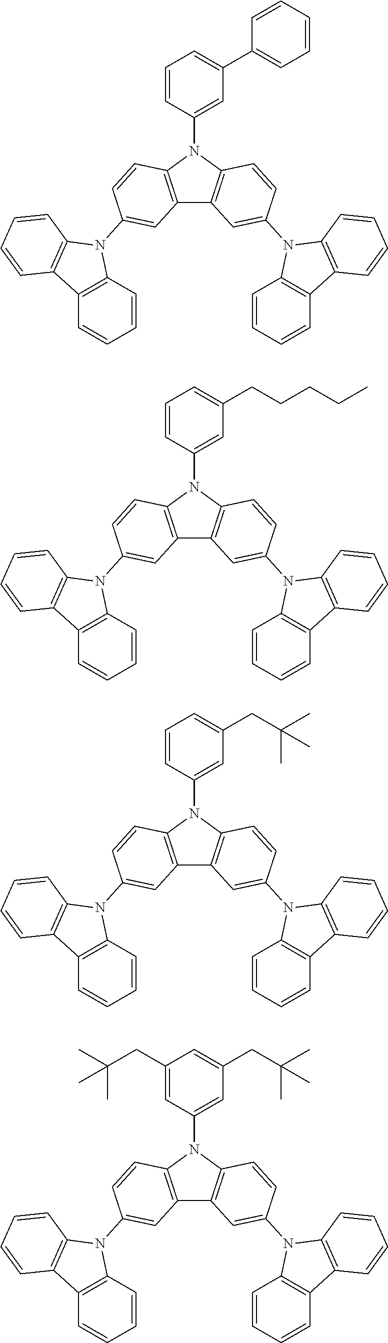

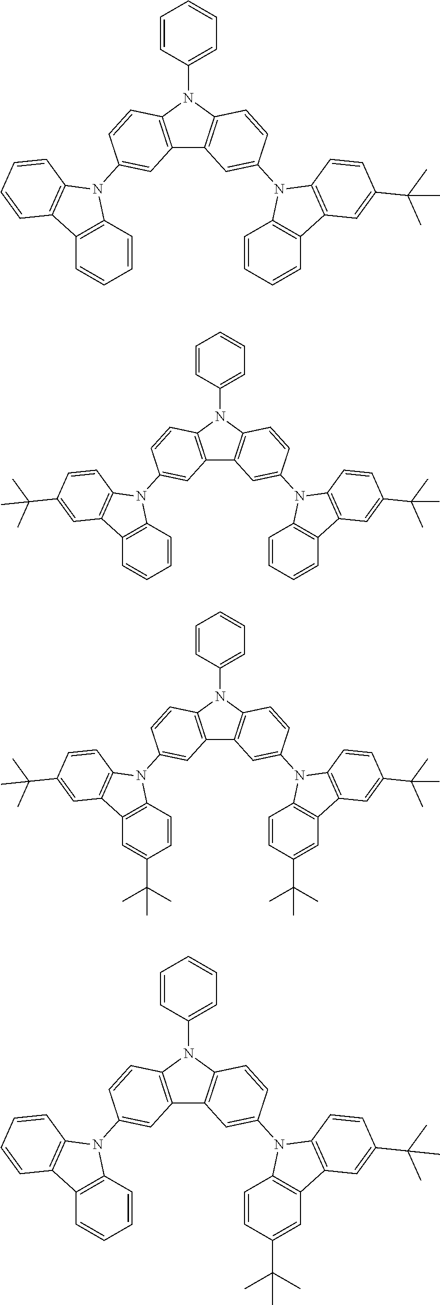

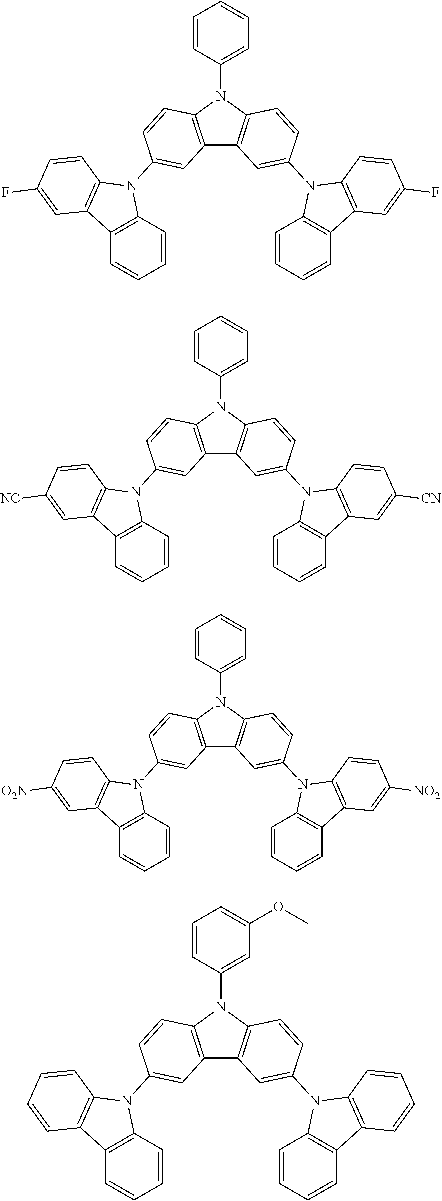

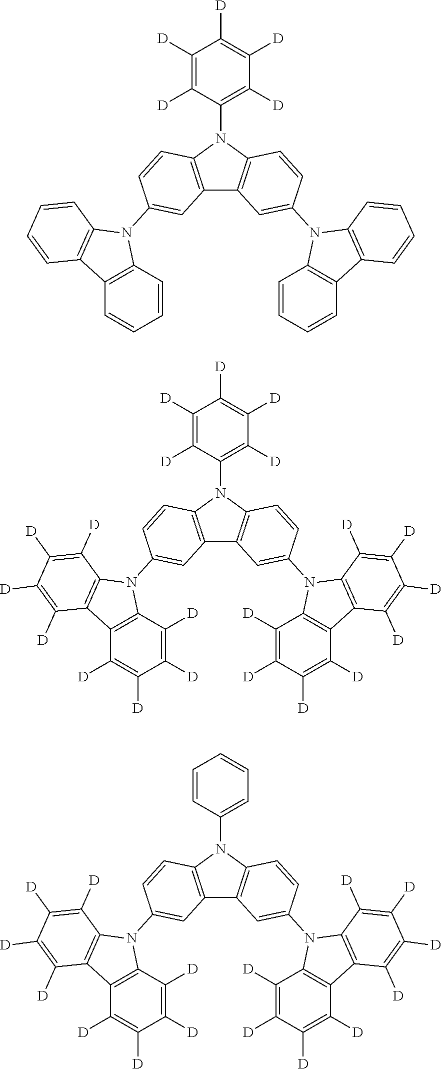

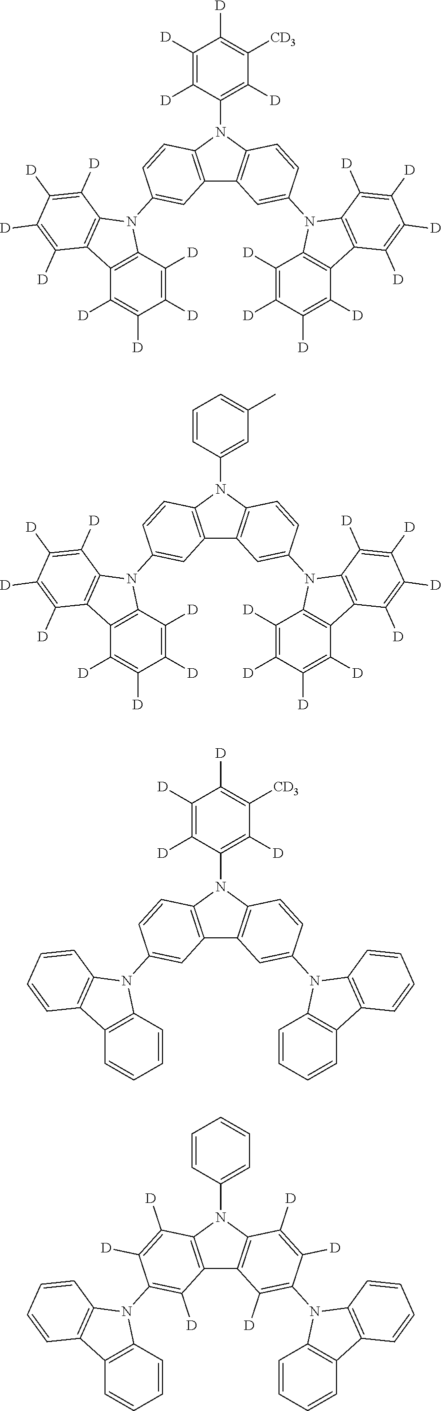

### —Electron-Injecting Layer, Electron-Transporting Layer—

The electron-injecting layer and the electron-transporting layer are layers having functions of receiving electrons from the cathode or cathode side and transporting the electrons to the anode side. The electron-injecting materials and the electron-transporting materials used in these layers may be low molecular weight compounds or high molecular weight compounds.

The compound represented by formula (1) of the invention can be used as the electron-transporting material. These layers are preferably layers containing, besides the compounds of the invention, pyridine derivatives, quinoline derivatives, pyrimidine derivatives, pyrazine derivatives, phthalazine derivatives, phenanthroline derivatives, triazine derivatives, triazole derivatives, oxazole derivatives, oxadiazole derivatives, imidazole derivatives, fluorenone derivatives, anthraquinodimethane derivatives, anthrone derivatives, diphenylquinone derivatives, thiopyran dioxide derivatives, carbodiimide derivatives, fluorenylidenemethane derivatives, distyrylpyrazine derivatives, aromatic cyclic tetracarboxylic anhydrides such as naphthalene and perylene, phthalocyanine derivatives, metal complexes of 8-quinolinol derivatives, various metal complexes represented by metal complexes having metalphthalocyanine, benzoxazole or benzothiazole as a ligand, and organic silane derivatives represented by silole.

The thickness of each of the electron-injecting layer and electron-transporting layer is preferably 500 nm or less from the viewpoint of lowering driving voltage.

The thickness of the electron-transporting layer is preferably 1 nm to 500 nm, more preferably 5 nm to 200 nm, and still more preferably 10 nm to 100 nm. The thickness of the electron-injecting layer preferably 0.1 nm to 200 nm, more preferably 0.2 nm to 100 nm, and still more preferably 0.5 nm to 50 nm.

The electron-injecting layer and electron-transporting layer may have a monolayer structure containing one or two or more kinds of the above materials, or may have a multilayer structure comprising two or more layers of the same composition or different compositions.

### —Hole-Blocking Layer—

The hole-blocking layer is a layer having a function of preventing the holes transported from the anode side to the light-emitting layer from passing through to the cathode side. In the invention, the hole-blocking layer can be provided as an organic layer contiguous to the light-emitting layer on the cathode side.

As the examples of organic compounds for constituting the hole-blocking layer, aluminum complexes such as aluminum(III)bis(2-methyl-8-quinolinato)-4-phenylphenolate (abbreviated to BAlq), triazole derivatives, and phenanthroline derivatives such as 2,9-dimethyl-4,7-diphenyl-1,10-phenanthroline (abbreviated to BCP) can be exemplified.

The thickness of the hole-blocking layer is preferably 1 nm to 500 nm, more preferably 5 nm to 200 nm, and still more preferably 10 nm to 100 nm.

The hole-blocking layer may have a monolayer structure containing one or two or more kinds of the above materials, or may be a multilayer structure comprising two or more layers of the same composition or different compositions.

### —Electron-Blocking Layer—

The electron-blocking layer is a layer having a function of preventing the electrons transported from the cathode side to the light-emitting layer from passing through to the anode side. In the invention, the electron-blocking layer can be provided as an organic layer contiguous to the light-emitting layer on the anode side.

As the examples of organic compounds for constituting the electron-blocking layer, for example, the hole-transporting materials described above can be applied.

The thickness of the electron-blocking layer is preferably 1 nm to 500 nm, more preferably 5 nm to 200 nm, and still more preferably 10 nm to 100 nm.

The electron-blocking layer may have a monolayer structure containing one or two or more kinds of the above materials, or may be a multilayer structure comprising two or more layers of the same composition or different compositions.

### <Protective Layer>

In the invention, the organic EL device may be entirely protected with a protective layer.

Concerning the protective layer, the items described in JP-A-2008-270736, paragraphs [0169] to [0170] can be applied to the invention.

### <Substrate>

The substrate for use in the invention is preferably a substrate that does not scatter or attenuate the light emitted from the organic layers.

### <Anode>

The anode is generally sufficient to have a function of the electrode to supply holes to organic layers. The shape, structure and size of the anode are not especially restricted, and the anode can be arbitrarily selected from known materials of electrode in accordance with the intended use and purpose of the light-emitting device. The anode is generally provided as the transparent anode.

### <Cathode>

The cathode is generally sufficient to have a function of the electrode to inject electrons to organic layers. The shape, structure and size of the cathode are not especially restricted, and the cathode can be arbitrarily selected from known materials of electrode in accordance with the intended use and purpose of the light-emitting device.

Concerning the substrate, anode and cathode, the items described in JP-A-2008-270736, paragraphs [0070] to [0089] can be applied to the invention.

### <Sealing Case>

The device in the invention may be entirely sealed with a sealing case.

Concerning the sealing case, the items described in JP-A-2008-270736, paragraph [0071] can be applied to the invention.

### (Driving)

By the application of D.C. (if necessary, A.C. component may be contained) voltage (generally 2 to 15 volts) between the anode and the cathode, or by the application of D.C. electric current, light emission of the organic electroluminescence device of the invention can be obtained.

With respect to the driving method of the organic electroluminescence device of the invention, the driving methods disclosed in JP-A-2-148687, JP-A-6-301355, JP-A-5-29080, JP-A-7-134558, JP-A-8-234685, JP-A-8-241047, Patent 2784615, U.S. Pat. Nos. 5,828,429 and 6,023,308 can be applied to the invention.

The external quantum efficiency of the organic electroluminescence device according to the invention is preferably 7% or more, more preferably 10% or more, and still more preferably 12% or more. As the numerical value of external quantum efficiency, the maximum value of external quantum efficiency at the time of driving the device at 20° C., alternatively the value of external quantum efficiency near 300 to 400 cd/m² at the time of driving the device at 20° C., can be used.

The internal quantum efficiency of the organic electroluminescence device in the invention is preferably 30% or more, more preferably 50% or more, and still more preferably 70% or more. The internal quantum efficiency of the device is computed by dividing the external quantum efficiency by the light collecting efficiency. The light collecting efficiency of ordinary organic EL devices is about 20%, but it is possible to make the light collecting efficiency 20% or more by variously designing the shape of substrate, the shape of electrode, the thickness of organic layer, the thickness of inorganic layer, the refractive index of organic layer, the refractive index of inorganic layer, etc.

### (Use of the Device of the Invention)

The device in the invention can be preferably used in display devices, displays, backlights, electrophotography, light sources for illumination, light sources for recording, light sources for exposure, light sources for reading, indicators, signboards, interior designs, optical communications, and the like. The devices in the invention are particularly preferably used in devices driven in the region high in light emission luminance such as illumination apparatus and display apparatus.

In the next place, the light emission apparatus in the invention is described with referring to FIG. 2.

FIG. 2 is a cross-sectional view schematically showing an example of the light emission apparatus according to the invention. Light emission apparatus 20 in FIG. 2 consists of transparent substrate (a supporting substrate) 2, organic electroluminescence device 10, and sealing case 16.

Organic electroluminescence device 10 comprises substrate 2 having thereon laminated anode (first electrode) 3, organic layer 11, and cathode (second electrode) 9 in this order. On cathode 9 is laminated protective layer 12, and sealing case 16 is provided on protective layer 12 sandwiching adhesive layer 14 in. In FIG. 2, parts of electrodes 3 and 9, bulkhead and insulating layer are omitted.

As adhesive layer 14, photo-curable adhesives such as epoxy resin and the like and thermosetting adhesives can be used and, for example, a thermosetting adhesive sheet can also be used.

The uses of the light emission apparatus in the invention are not especially restricted and, besides illuminating apparatus, the light emission apparatus can be used, for example, as display apparatus such as television, personal computer, portable telephone, electronic paper, and the like.

### (Illumination Apparatus)

In the next place, the illumination apparatus of the invention is described with referring to FIG. 3.

FIG. 3 is a cross-sectional view schematically showing an example of the illumination apparatus according to the invention. Illumination apparatus 40 in the invention is equipped with organic EL device 10 and light-scattering member 30, as shown in FIG. 3. More specifically, illumination apparatus 40 is constituted so that substrate 2 of organic EL device 10 is contiguous to light-scattering member 30.

Illumination apparatus 40 in the exemplary embodiment of the invention is equipped with organic EL device 10 and light-scattering member 30, as shown in FIG. 3. More specifically, illumination apparatus 40 is constituted so that substrate 2 of organic EL device 10 is contiguous to light-scattering member 30.

Light-scattering member 30 is not especially restricted so long as it can scatter light, but in FIG. 3, light-scattering member 30 is a member comprising transparent substrate 31 containing particles 32 having been dispersed therein. As transparent substrate 31, e.g., a glass substrate is preferably exemplified. As particles 32, transparent resin particles are preferably exemplified. As the glass substrate and transparent resin particles, known materials can be used. In illumination apparatus 40, when light emission from organic electroluminescence device 10 is incident to plane of incidence of light 30A of light-scattering member 30, the incident light is scattered by light-scattering member 30, and the scattered light is outgoing from plane of outgoing of light 30B as illumination light.

### EXAMPLES

The invention will be described in further detail with reference to examples, but the invention is by no means restricted thereto. In particular, the presence or absence of substituents hardly affects the advantage of the invention, and the similar results can be obtained even when the compounds used in the following examples have substituents.

### Example 1

### Synthesis of Exemplified Compound 1

### (Synthesis Method B: Comparative Synthesis Method)

Exemplified compound 1 of the charge-transporting material represented by formula (1) was synthesized and purified according to the method described in WO 05/085387, paragraphs [0074] to [0075]. The reaction formulae are shown below.

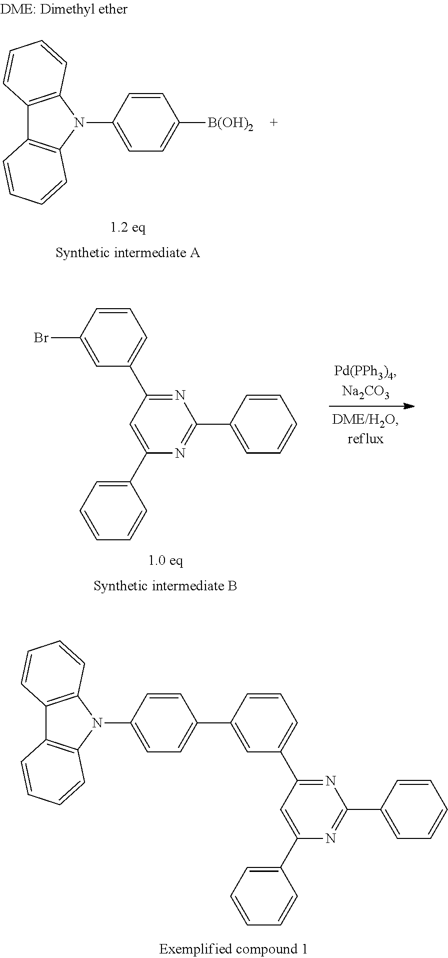

The obtained sample was subjected to sublimation purification (heating at 5×10⁻¹ Pa under Ar flow), and fractions A, B and C were portioned out from the settled position of the sample at the time of recovery, from which each charge-transporting material was obtained. Fraction B is a region farther than fraction A and fraction C is a region farther than fraction B from the settled position of the sample. A material capable of vaporizing at a lower temperature is recovered from fraction B as compared with fraction A, and from fraction C as compared with fraction B.

Purification by HPLC and the contents of specific impurities of the obtained charge-transporting material are shown in Table 1 below together with the characteristics of device. In Table 1, the material not subjected to sublimation purification was inscribed as “No Sublimation”.

### (Synthesis Method A: Synthesis Method of the Invention)

Exemplified compound 1 was synthesized and purified with the same molar concentration of the catalyst, molar concentration of the solvent, molar concentration of the base, reaction condition and purification condition as in synthesis method B except for changing synthesis intermediate A to synthesis intermediate M-1 and synthesis intermediate B to synthesis intermediate M-2. The reaction formulae are shown below.

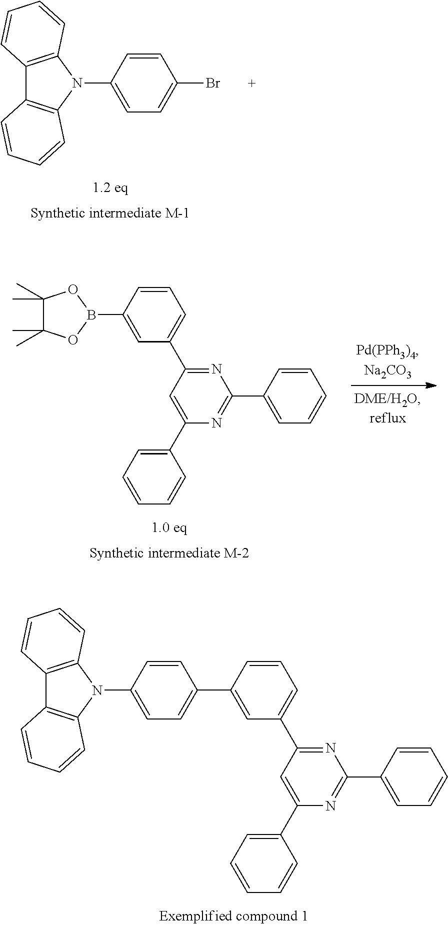

The obtained sample was subjected to sublimation purification in the same manner as in synthesis method B, and fractions A, B and C were portioned out, from which each charge-transporting material was obtained. Fraction B is a region farther than fraction A and fraction C is a region farther than fraction B from the settled position of the sample. A material capable of vaporizing at a lower temperature is recovered from fraction B as compared with fraction A, and from fraction C as compared with fraction B.

Purification by HPLC and the contents of specific impurities of the obtained charge-transporting material are shown in Table 1 below together with the characteristics of device.

Impurity 1 in Table 1 is aryl halide having a carbazole part, which is a compound corresponding to the compound represented by formula (I-1) or (II-1) of the invention. In the case of exemplified compound 1 of the charge-transporting material, synthesis intermediate M-1 also corresponds thereto. Impurity 2 is aryl halide having a pyrimidine part, which is a compound corresponding to the compound represented by formula (I-2) or (II-2) of the invention. In exemplified compound 1 of the charge-transporting material, synthesis intermediate B also corresponds thereto.

Exemplified compounds 5, 6, 20 and 36 of the charge-transporting material represented by formula (1) were also synthesized and purified similarly to the synthesis of exemplified compound 1. The compounds synthesized by the synthesis method of the invention are inscribed as synthesis method A and those synthesized according to the methods described in WO 05/085387 and WO 03/080760 or methods following these methods as synthesis method B. Exemplified compounds 20 and 36 were synthesized by coupling the compound corresponding to formula (I-2) or (II-2) of the invention with carbazole, which method is defined as the method corresponding to synthesis method B. The structures of impurity 1 and impurity 2 in the syntheses of exemplified compounds 5, 6, 20 and 36 are shown below.

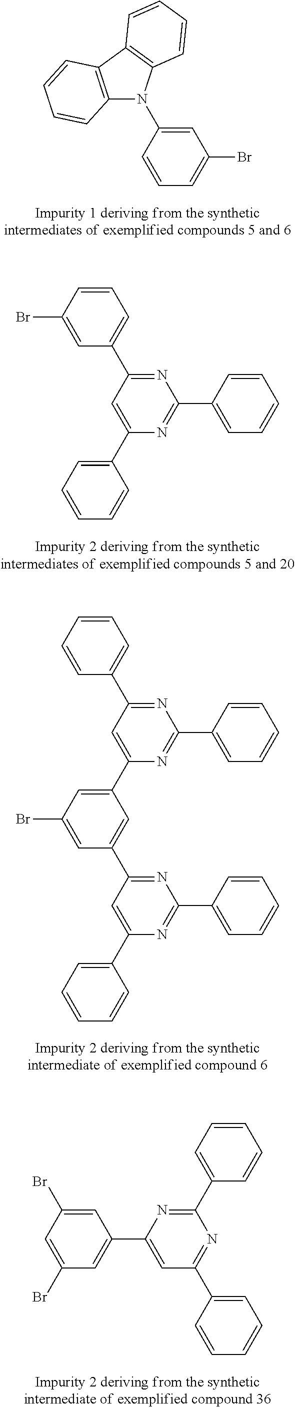

Similarly, exemplified compounds 37, 38, 40, 41, 42, 45, 46, 47, 50, 51, 52, 53, 54 and 55 of formula (1) were synthesized by synthesis method A or B. The structures of impurity 1 and impurity 2 of exemplified compounds 37, 38, 40, 41, 42, 45, 46, 47, 50, 51, 52, 53, 54 and 55 are shown below.

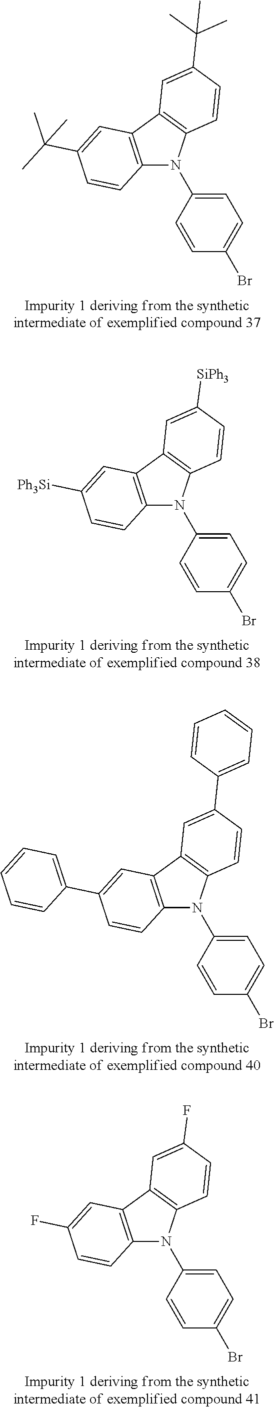

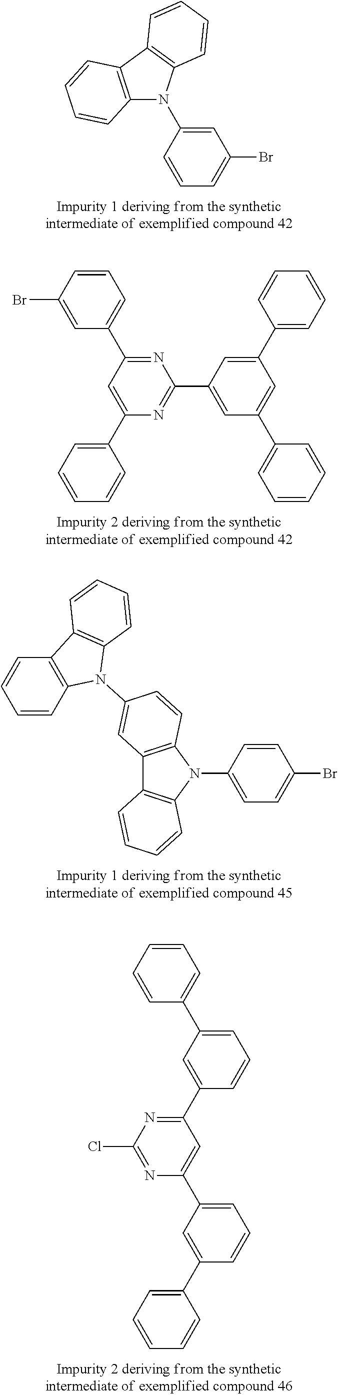

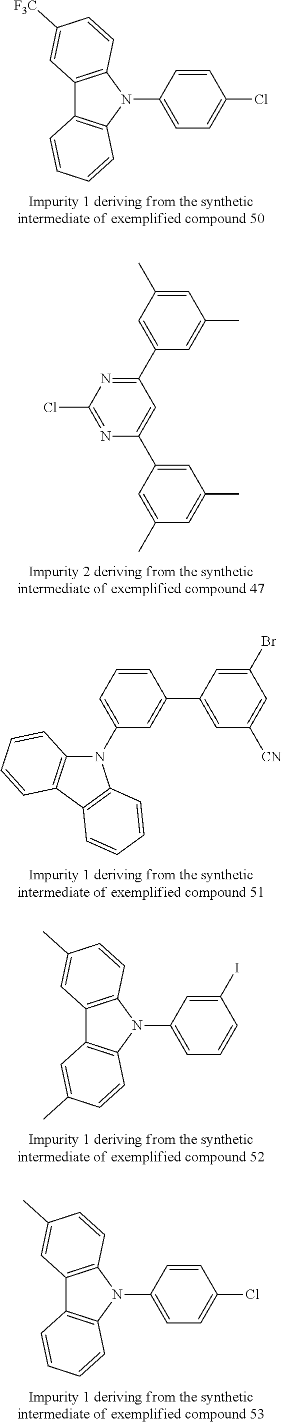

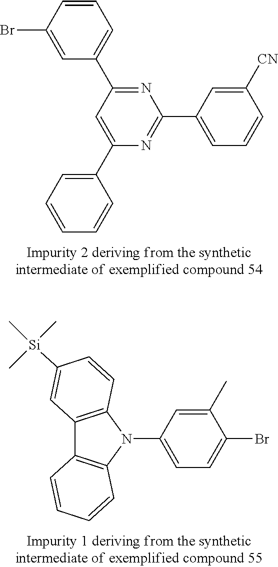

### Example 2

### Manufacture of Device

A glass substrate having an ITO film (manufactured by Geomatec Co., Ltd., surface resistance: 10Ω/□)) having a thickness of 0.5 mm and a size of 2.5 cm square was put in a washer and subjected to ultrasonic washing in 2-propanol, and then UV-ozone treatment for 30 minutes. The organic compound layers shown below were deposited on the transparent anode (ITO film) in sequence by a vacuum deposition method.

First Layer: 2-TNATA and F₄-TCNQ (mass ratio: 99.7/0.3); film thickness: 120 nm
Second Layer: α-NPD; film thickness: 7 nm
Third Layer: C-1; film thickness: 3 nm
Fourth Layer: H-1 and D-1 (mass ratio: 85/15); film thickness: 30 nm
Fifth Layer Electron-transporting material (the charge-transporting material manufactured in Example 1, shown in Tables 1 and 2); film thickness: 3 nm
Sixth Layer: BAlq; film thickness: 27 nm

Lithium fluoride in a thickness of 0.1 nm and metal aluminum in a thickness of 100 nm were deposited thereon in this order to obtain a cathode.

The obtained product was put in a glove box replaced with nitrogen gas so as not to be in contact with air, and sealed with a glass sealing case and a UV-curable type adhesive (XNR5516HV, manufactured by Nagase Ciba Corp.) to obtain an organic electroluminescence device.

As a result of light emission of these devices, light emission resulting from the used light-emitting material was obtained from each device.

### (Performance Evaluation of Organic Electroluminescence Device)

Performances of each obtained device were evaluated by measuring external quantum efficiency and driving durability. The measurements were carried out as follows. The results obtained are shown in Tables 1 and 2.

### (a) External Quantum Efficiency

DC voltage was applied to each device for light emission with source measure unit Model 2400 (manufactured by Toyo Corporation). The luminance at that time was measured with a luminometer BM-8 (manufactured by Topcon Corporation). The light emission spectrum and emission wavelength were measured with a spectrum analyzer PMA-11 (manufactured by Hamamatsu Photonics K.K.). On the basis of these measurements, external quantum efficiency near 360 cd/m² of luminance was computed according to a luminance conversion method.

### (b) Driving Durability

DC voltage was applied to each device so as to reach luminance of 1,000 cd/m², and the time required to reach luminance of 500 cd/m² was measured. This half life time of luminance was taken as the index of evaluation of driving durability. Incidentally, the value of the device formed with the compound manufactured from exemplified compound 1 of the charge-transporting material synthesized by synthesis method A and used sublimation purification fraction A was taken as 1.0, and the value of each device was shown in Tables 1 and 2 as the relative value to this value.

TABLE 1

| | Electron | | Sublimation | Purity by | Content of | Content of | External Quantum | Driving |
|---|---|---|---|---|---|---|---|---|
| | Transporting | Synthesis | Purification | HPLC | Impurity 1 | Impurity 2 | Efficiency | Dura- |
| Device No. | Material | Method | Fraction | (% by mass) | (% by mass) | (% by mass) | (%) | bility |
| Device 1-1 of the Invention | Exemplified | A | A | >99.9 | <0.1 | <0.1 | 12 | 1.0 |
| Device 1-2 of the Invention | Compound 1 | | B | 99.7 | <0.1 | <0.1 | 12 | 1.0 |
| Device 1-3 of the Invention | | | C | 99.0 | <0.1 | <0.1 | 12 | 1.0 |
| Device 1-1 for Comparison | | | No sublimation. | 98.6 | 0.4 | <0.1 | 10 | 0.3 |
| Device 1-2 for Comparison | | B | A | 99.8 | <0.1 | 0.2 | 11 | 0.6 |
| Device 1-3 for Comparison | | | B | 99.4 | <0.1 | 0.6 | 11 | 0.3 |
| Device 1-4 for Comparison | | | No sublimation. | 97.9 | <0.1 | 1.5 | 10 | <0.1 |
| Device 1-4 of the Invention | Exemplified | A | A | >99.9 | <0.1 | <0.1 | 11 | 0.8 |
| Device 1-5 of the Invention | Compound 5 | | B | 99.8 | <0.1 | <0.1 | 11 | 0.8 |
| Device 1-5 for Comparison | | | No sublimation. | 99.0 | 0.4 | <0.1 | 9 | 0.3 |
| Device 1-6 for Comparison | | B | A | 99.7 | <0.1 | 0.3 | 10 | 0.5 |
| Device 1-7 for Comparison | | | B | 99.2 | <0.1 | 0.8 | 10 | 0.2 |
| Device 1-8 for Comparison | | | No sublimation. | 98.4 | <0.1 | 1.2 | 8 | <0.1 |
| Device 1-6 of the Invention | Exemplified | A | A | 99.6 | <0.1 | <0.1 | 13 | 0.7 |
| Device 1-7 of the Invention | Compound 6 | | B | 99.2 | <0.1 | <0.1 | 13 | 0.7 |
| Device 1-9 for Comparison | | | No sublimation. | 98.3 | 1.0 | <0.1 | 11 | <0.1 |
| Device 1-8 of the Invention | Exemplified | B | A | 99.7 | <0.1 | <0.1 | 7 | 0.5 |
| Device 1-10 for Comparison | Compound 20 | | B | 99.3 | <0.1 | 0.7 | 6 | <0.1 |
| Device 1-11 for Comparison | | | No sublimation. | 98.7 | <0.1 | 0.9 | 5 | <0.1 |
| Device 1-9 of the Invention | Exemplified | B | A | 99.5 | <0.1 | <0.1 | 7 | 0.6 |
| Device 1-12 for Comparison | Compound 36 | | B | 98.8 | <0.1 | 0.9 | 5 | <0.1 |
| Device 1-13 for Comparison | | | No sublimation. | 98.0 | <0.1 | 1.5 | 5 | <0.1 |
| Device 1-10 of the Invention | Exemplified | A | A | 99.5 | <0.1 | <0.1 | 13 | 0.7 |
| Device 1-11 of the Invention | Compound 37 | | B | 99.2 | <0.1 | <0.1 | 13 | 0.7 |
| Device 1-14 for Comparison | | | No sublimation. | 88.4 | 0.5 | <0.1 | 11 | <0.1 |
| Device 1-12 of the Invention | Exemplified | A | A | 99.7 | <0.1 | <0.1 | 13 | 0.7 |
| Device 1-13 of the Invention | Compound 38 | | B | 99.1 | <0.1 | <0.1 | 13 | 0.7 |
| Device 1-15 for Comparison | | | No sublimation. | 98.1 | 0.6 | <0.1 | 11 | <0.1 |
| Device 1-14 of the Invention | Exemplified | A | A | 99.5 | <0.1 | <0.1 | 13 | 0.7 |
| Device 1-15 of the Invention | Compound 40 | | B | 99.4 | <0.1 | <0.1 | 13 | 0.7 |
| Device 1-16 for Comparison | | | No sublimation. | 98.2 | 0.4 | <0.1 | 11 | <0.1 |
| Device 1-16 of the Invention | Exemplified | A | A | 99.5 | <0.1 | <0.1 | 13 | 0.7 |
| Device 1-17 of the Invention | Compound 41 | | B | 99.3 | <0.1 | <0.1 | 13 | 0.7 |
| Device 1-17 for Comparison | | | No sublimation. | 98.1 | 0.9 | <0.1 | 11 | <0.1 |
| Device 1-18 of the Invention | Exemplified | A | A | 99.6 | <0.1 | <0.1 | 13 | 0.7 |
| Device 1-19 of the Invention | Compound 42 | | B | 99.2 | <0.1 | <0.1 | 13 | 0.7 |
| Device 1-20 for Comparison | | | No sublimation. | 98.2 | 1.0 | <0.1 | 11 | <0.1 |

TABLE 2

| | Electron | | Sublimation | Purity by | Content of | Content of | External Quantum | Driving |
|---|---|---|---|---|---|---|---|---|
| | Transporting | Synthesis | Purification | HPLC | Impurity 1 | Impurity 2 | Efficiency | Dura- |
| Device No. | Material | Method | Fraction | (% by mass) | (% by mass) | (% by mass) | (%) | bility |
| Device 1-20 of the Invention | Exemplified | A | A | >99.9 | <0.1 | <0.1 | 12 | 1.2 |
| Device 1-21 of the Invention | Compound 45 | | B | 99.5 | <0.1 | <0.1 | 12 | 1.2 |
| Device 1-21 for Comparison | | | No sublimation. | 99.2 | 0.3 | <0.1 | 11 | 0.4 |
| Device 1-22 of the Invention | Exemplified | B | A | 99.9 | <0.1 | <0.1 | 7 | 0.7 |
| Device 1-22 for Comparison | Compound 46 | | B | 99.6 | <0.1 | 0.2 | 7 | 0.3 |
| Device 1-23 for Comparison | | | No sublimation. | 99.0 | <0.1 | 0.4 | 7 | 0.2 |
| Device 1-23 of the Invention | Exemplified | B | A | 99.9 | <0.1 | <0.1 | 7 | 0.6 |
| Device 1-24 of the Invention | Compound 47 | | B | 99.7 | <0.1 | <0.1 | 7 | 0.6 |
| Device 1-24 for Comparison | | | C | 99.0 | <0.1 | 0.3 | 6 | 0.2 |
| Device 1-25 for Comparison | | | No sublimation. | 98.4 | <0.1 | 1.1 | 6 | <0.1 |
| Device 1-25 of the Invention | Exemplified | A | A | >99.9 | <0.1 | <0.1 | 10 | 0.8 |
| Device 1-26 of the Invention | Compound 50 | | B | 99.7 | <0.1 | <0.1 | 10 | 0.8 |
| Device 1-26 for Comparison | | | No sublimation. | 99.3 | 0.3 | <0.1 | 10 | 0.2 |
| Device 1-27 of the Invention | Exemplified | A | A | 99.6 | <0.1 | <0.1 | 13 | 1.1 |
| Device 1-28 of the Invention | Compound 51 | | B | 99.4 | <0.1 | <0.1 | 13 | 1.1 |
| Device 1-27 for Comparison | | | No sublimation. | 98.0 | 1.3 | <0.1 | 12 | <0.1 |
| Device 1-29 of the Invention | Exemplified | A | A | 99.9 | <0.1 | <0.1 | 9 | 0.8 |
| Device 1-30 of the Invention | Compound 52 | | B | 99.8 | <0.1 | <0.1 | 9 | 0.8 |
| Device 1-28 for Comparison | | | C | 99.4 | 0.2 | <0.1 | 9 | <0.1 |
| Device 1-29 for Comparison | | | No sublimation. | 99.1 | 0.5 | <0.1 | 8 | <0.1 |
| Device 1-31 of the Invention | Exemplified | A | A | 99.7 | <0.1 | <0.1 | 11 | 0.8 |
| Device 1-32 of the Invention | Compound 53 | | B | 99.7 | <0.1 | <0.1 | 11 | 0.8 |
| Device 1-30 for Comparison | | | No sublimation. | 99.0 | 0.8 | <0.1 | 11 | 0.2 |
| Device 1-33 of the Invention | Exemplified | B | A | 99.5 | <0.1 | <0.1 | 9 | 1.1 |
| Device 1-31 for Comparison | Compound 54 | | B | 99.0 | <0.1 | 0.3 | 8 | 0.2 |
| Device 1-32 for Comparison | | | No sublimation. | 98.7 | <0.1 | 0.5 | 8 | <0.1 |
| Device 1-34 of the Invention | Exemplified | A | A | >99.9 | <0.1 | <0.1 | 8 | 0.9 |
| Device 1-35 of the Invention | Compound 55 | | B | 99.7 | <0.1 | <0.1 | 8 | 0.9 |
| Device 1-33 for Comparison | | | No sublimation. | 99.1 | 0.5 | <0.1 | 7 | <0.1 |

By the comparison of the devices using the same electron-transporting material from the results shown in Tables 1 and 2, it can be seen that the devices of the invention suppressing the content of impurities 1 and 2 to 0.1% by mass or less are excellent both in light emission efficiency and durability.

Further, it is understood that electron-transporting materials capable of providing devices excellent in light emission efficiency and durability can be obtained when compounds of electron-transporting materials are synthesized according to the synthesis method of the invention regardless of the position of fraction in sublimation purification after synthesis.

### Example 3

### Manufacture of Device

Each device was manufactured in the same manner as in Example 2 except for carrying out deposition of the organic compound layers in the order of the following first layer to fifth layer.

First Layer: 2-TNATA and F₄-TCNQ (mass ratio: 99.7/0.3); film thickness: 120 nm
Second Layer: α-NPD; film thickness: 7 nm
Third Layer: C-1; film thickness: 3 nm
Fourth Layer Host material as shown in Tables 3 and 4 (the charge-transporting material manufactured in Example 1) and light-emitting material (mass ratio: 95/5); film thickness: 30 nm
Fifth Layer: BAlq; film thickness: 30 nm

As a result of light emission of these devices, light emission resulting from the used light-emitting material was obtained from each device. Performances of each obtained device were evaluated by measuring external quantum efficiency and driving durability in the same manner as in Example 2. The results obtained are shown in Tables 3 and 4.

Concerning the value of driving durability shown in Tables 3 and 4, the value of the device formed with the compound manufactured from exemplified compound 1 of the charge-transporting material synthesized by synthesis method A and used sublimation purification fraction A was taken as 1.0, and the value of each device was shown in Tables 3 and 4 below as the relative value to this value.

TABLE 3

| | | | Sublimation | Purity by | Content of | Content of | External Quantum | Driving |
|---|---|---|---|---|---|---|---|---|
| | | Synthesis | Purification | HPLC | Impurity 1 | Impurity 2 | Efficiency | Dura- |
| Device No. | Material of Fourth Layer | Method | Fraction | (% by mass) | (% by mass) | (% by mass) | (%) | bility |
| Device 2-1 of | Host material: Exemplified | A | A | >99.9 | <0.1 | <0.1 | 8 | 1.0 |
| the Invention | Compound 1 | | | | | | | |
| Device 2-2 of | Light-emitting material: D-1 | | C | 99.0 | <0.1 | <0.1 | 8 | 1.0 |
| the Invention | | | | | | | | |
| Device 2-1 for | | B | A | 99.8 | <0.1 | 0.2 | 7 | 0.7 |
| Comparison | | | | | | | | |
| Device 2-3 of | Host material: Exemplified | A | A | 99.6 | <0.1 | <0.1 | 8 | 0.5 |
| the Invention | Compound 6 | | | | | | | |
| Device 2-2 for | Light-emitting material: D-1 | | No sublimation. | 98.3 | 1.0 | <0.1 | 7 | <0.1 |
| Comparison | | | | | | | | |

TABLE 4

| | | | Sublimation | Purity by | Content of | Content of | External Quantum | Driving |
|---|---|---|---|---|---|---|---|---|
| | | Synthesis | Purification | HPLC | Purity 1 | Purity 2 | Efficiency | Dura- |
| Device No. | Material of Fourth Layer | Method | Fraction | (% by mass) | (% by mass) | (% by mass) | (%) | bility |
| Device 2-4 of | Host material: Exemplified | A | A | >99.9 | <0.1 | <0.1 | 16 | 23 |
| the Invention | Compound 1 | | | | | | | |
| Device 2-5 of | Light-emitting material: D-2 | | C | 99.0 | <0.1 | <0.1 | 16 | 23 |
| the Invention | | | | | | | | |
| Device 2-3 for | | B | A | 99.6 | <0.1 | 0.2 | 14 | 8.0 |
| Comparison | | | | | | | | |

From the results shown in Tables 3 and 4, it can be seen that the devices of the invention suppressing the content of impurities 1 and 2 to 0.1% by mass or less are excellent both in light emission efficiency and durability even when the electron transporting material of the invention is used as the host material in the light-emitting layer.

Devices were manufactured in the same manner as in Example 2 except for changing the third layer, fourth layer and fifth layer as shown in the following Tables 5 and 7, and evaluated in the same manner as in Example 2. The results obtained are shown in Tables 6 and 8 below. Incidentally, the synthesis method of the used electron-transporting material of the invention and sublimation purification fraction are inscribed as exemplified compound 1 (#A-B) (which means synthesis method A, sublimation purification fraction B).

The maximum light emission wavelength of each device as shown in Tables 5 and 7 was measured with a spectrum analyzer PMA-11 (manufactured by Hamamatsu Photonics K.K.). The value of DC voltage at the time when luminance reaches 1,000 cd/m² is driving voltage.

The ratio in the parenthesis shown in the column of “Fourth Layer” in Tables 5 and 7 is the ratio by mass of the host material and the light-emitting material. Further, light-emitting materials 2-1 to 2-3, 2-6, 2-8, 3-2 to 3-5, 5-3, 5-4, 8-4, 9-6, 9-17 and 9-19 are the compounds respectively with the same numbers shown in the specification.

TABLE 5

| | | | | Maximum |
|---|---|---|---|---|
| | | | | Light |
| | | | | Emission |
| | Third | | | Wavelength |
| Device No. | Layer | Fourth Layer | Fifth Layer | (nm) |
| Device 3-1 of | C-2 | H-2/2-1 (85/15) | Exemplified | 511 |
| the Example | | | Compound 1 | |
| | | | (#A-A) | |
| Device 3-2 of | C-2 | H-4/2-2 (90/10) | Exemplified | 492 |
| the Example | | | Compound 1 | |
| | | | (#A-A) | |
| Device 3-3 of | C-6 | H-8/2-3 (87/13) | Exemplified | 505 |
| the Example | | | Compound 5 | |
| | | | (#A-A) | |
| Device 3-4 of | C-3 | H-5/2-6 (90/10) | Exemplified | 528 |
| the Example | | | Compound 5 | |
| | | | (#A-A) | |
| Device 3-5 of | C-2 | H-3/2-8 (85/15) | Exemplified | 501 |
| the Example | | | Compound 6 | |
| | | | (#A-A) | |
| Device 3-6 of | C-2 | H-6/3-2 (85/15) | Exemplified | 469 |
| the Example | | | Compound 1 | |
| | | | (#A-A) | |
| Device 3-7 of | C-4 | H-7/3-3 (80/20) | Exemplified | 466 |
| the Example | | | Compound 1 | |
| | | | (#A-A) | |
| Device 3-8 of | C-5 | H-2/3-4 (90/10) | Exemplified | 464 |
| the Example | | | Compound 1 | |
| | | | (#A-A) | |
| Device 3-9 of | C-2 | H-3/3-5 (85/15) | Exemplified | 531 |
| the Example | | | Compound 6 | |
| | | | (#A-A) | |
| Device 3-10 of | C-2 | H-2/8-4 (85/15) | Exemplified | 456 |
| the Example | | | Compound 20 | |
| | | | (#B-A) | |
| Device 3-11 of | C-3 | H-6/9-6 (85/15) | Exemplified | 483 |
| the Example | | | Compound 5 | |
| | | | (#A-A) | |
| Device 3-12 of | C-5 | H-9/9-17 (97/3) | Exemplified | 467 |
| the Example | | | Compound 5 | |
| | | | (#A-A) | |
| Device 3-13 of | C-2 | H-7/9-19 (85/15) | Exemplified | 538 |
| the Example | | | Compound 5 | |
| | | | (#A-A) | |
| Device 3-14 of | C-2 | H-9/Exemplified | BAlq | 505 |
| the Example | | Compound 1 | | |
| | | (#A-A)/2-3 | | |
| | | (70/15/15) | | |
| Device 3-15 of | C-3 | H-5/FR-1 (95/5) | Exemplified | 608 |
| the Example | | | Compound 36 | |
| | | | (#B-A) | |
| Device 3-16 of | C-6 | H-3/FR-2 (93/7) | Exemplified | 603 |
| the Example | | | Compound 40 | |
| | | | (#A-A) | |
| Device 3-17 of | C-2 | H-5/FR-3 (93/7) | Exemplified | 631 |
| the Example | | | Compound 37 | |
| | | | (#A-A) | |

TABLE 6

| | External | | |
|---|---|---|---|
| | Quantum | Driving | |
| | Efficiency | Voltage | |
| Device No. | (%) | (V) | Durability |
| Device 3-1 of the Example | 14 | 5.1 | 32 |
| Device 3-2 of the Example | 14 | 5.4 | 25 |
| Device 3-3 of the Example | 17 | 4.7 | 55 |
| Device 3-4 of the Example | 16 | 4.9 | 41 |
| Device 3-5 of the Example | 16 | 4.8 | 27 |
| Device 3-6 of the Example | 11 | 5.5 | 0.8 |
| Device 3-7 of the Example | 12 | 5.3 | 0.7 |
| Device 3-8 of the Example | 8 | 5.7 | 0.5 |
| Device 3-9 of the Example | 15 | 5.2 | 51 |
| Device 3-10 of the Example | 9 | 5.9 | 0.6 |
| Device 3-11 of the Example | 12 | 5.4 | 3 |
| Device 3-12 of the Example | 10 | 5.8 | 0.7 |
| Device 3-13 of the Example | 16 | 5.0 | 49 |
| Device 3-14 of the Example | 17 | 4.5 | 51 |
| Device 3-15 of the Example | 15 | 4.4 | 98 |
| Device 3-16 of the Example | 16 | 4.0 | 87 |
| Device 3-17 of the Example | 15 | 4.1 | 94 |

TABLE 7

| | | | | Maximum |
|---|---|---|---|---|
| | | | | Light |
| | | | | Emission |
| | Third | | | Wavelength |
| Device No. | Layer | Fourth Layer | Fifth Layer | (nm) |
| Device 3-18 of | C-1 | H-1/5-3 | Exemplified | 480 |
| the Invention | | (85/15) | Compound 1 | |
| | | | (#A-A) | |
| Device 3-19 of | C-2 | H-8/5-3 | Exemplified | 480 |
| the Invention | | (85/15) | Compound 5 | |
| | | | (#A-A) | |
| Device 3-20 of | C-1 | H-2/5-4 | Exemplified | 485 |
| the Invention | | (85/15) | Compound 1 | |
| | | | (#A-A) | |
| Device 3-21 of | C-3 | H-5/5-4 | Exemplified | 485 |
| the Invention | | (85/15) | Compound 6 | |
| | | | (#A-A) | |
| Device 3-22 of | C-5 | H-3/FR-8 | Exemplified | 610 |
| the Invention | | (90/10) | Compound 1 | |
| | | | (#A-A) | |
| Device 3-23 of | C-4 | H-6/FR-8 | Exemplified | 610 |
| the Invention | | (90/10) | Compound 37 | |
| | | | (#A-A) | |
| Device 3-24 of | C-1 | H-7/FR-8 | Exemplified | 610 |
| the Invention | | (90/10) | Compound 41 | |
| | | | (#A-A) | |
| Device 3-25 of | C-2 | H-2/FR-26 | Exemplified | 615 |
| the Invention | | (90/10) | Compound 1 | |
| | | | (#A-A) | |
| Device 3-26 of | C-2 | H-3/FR-26 | Exemplified | 615 |
| the Invention | | (85/15) | Compound 20 | |
| | | | (#B-A) | |
| Device 3-27 of | C-4 | H-2/FR-36 | Exemplified | 610 |
| the Invention | | (85/15) | Compound 5 | |
| | | | (#A-A) | |
| Device 3-28 of | C-6 | H-6/FR-36 | Exemplified | 610 |
| the Invention | | (85/15) | Compound 41 | |
| | | | (#A-A) | |
| Device 3-29 of | C-2 | H-9/FR-37 | Exemplified | 610 |
| the Invention | | (97/3) | Compound 20 | |
| | | | (#B-A) | |
| Device 3-30 of | C-1 | H-7/FR-37 | Exemplified | 610 |
| the Invention | | (85/15) | Compound 42 | |
| | | | (#A-A) | |
| Device 3-31 of | C-1 | BAlq/FR-8 | Exemplified | 610 |
| the Invention | | (95/5) | Compound 1 | |
| | | | (#A-A) | |
| Device 3-1 for | C-1 | BAlq/FR-8 | Exemplified | 610 |
| Comparison | | (95/5) | Compound 1 | |
| | | | (#B-A) | |
| Device 3-32 of | C-2 | BAlq/FR-26 | Exemplified | 615 |
| the Invention | | (95/5) | Compound 37 | |
| | | | (#A-A) | |
| Device 3-2 for | C-2 | BAlq/FR-26 | Exemplified | 615 |
| Comparison | | (95/5) | Compound 1 | |
| | | | (#B-A) | |
| Device 3-35 of | C-1 | Exemplified | BAlq | 610 |
| the Invention | | Compound 1 | | |
| | | (#A-A)/FR-8 | | |
| | | (90/10) | | |
| Device 3-36 of | C-2 | Exemplified | Exemplified | 610 |
| the Invention | | Compound 1 | Compound 1 | |
| | | (#A-A)/FR-8 | (#A-A) | |
| | | (90/10) | | |
| Device 3-37 of | C-1 | Exemplified | ET-1 | 610 |
| the Invention | | Compound 1 | | |
| | | (#A-A)/FR-8 | | |
| | | (95/5) | | |
| Device 3-3 for | C-1 | Exemplified | ET-1 | 610 |
| Comparison | | Compound 1 | | |
| | | (#B-A)/FR-8 | | |
| | | (95/5) | | |
| Device 3-38 of | C-2 | Exemplified | ET-1 | 610 |
| the Invention | | Compound 45 | | |
| | | (#A-A)/FR-26 | | |
| | | (95/5) | | |
| Device 3-4 for | C-2 | Exemplified | ET-1 | 610 |
| Comparison | | Compound 45 | | |
| | | (#A-No | | |
| | | sublimation)/ | | |
| | | FR-26 (95/5) | | |
| Device 3-39 of | C-6 | Exemplified | BAlq | 615 |
| the Invention | | Compound 20 | | |
| | | (#B-A)/FR-26 | | |
| | | (90/10) | | |
| Device 3-40 of | C-3 | Exemplified | Exemplified | 615 |
| the Invention | | Compound 37 | Compound 37 | |
| | | (#A-A)/FR-26 | (#A-A) | |
| | | (90/10) | | |
| Device 3-41 of | C-2 | Exemplified | Exemplified | 610 |
| the Invention | | Compound 5 | Compound 1 | |
| | | (#A-A)/FR-36 | (#A-A) | |
| | | (93/7) | | |
| Device 3-42 of | C-4 | Exemplified | Exemplified | 610 |
| the Invention | | Compound 20 | Compound 41 | |
| | | (#B-A)/FR-37 | (#A-A) | |
| | | (93/7) | | |

TABLE 8

| | External | | |
|---|---|---|---|
| | Quantum | Driving | |
| | Efficiency | Voltage | |
| Device No. | (%) | (V) | Durability |
| Device 3-18 of the Invention | 14 | 5.1 | 7 |
| Device 3-19 of the Invention | 14 | 5.2 | 6 |
| Device 3-20 of the Invention | 14 | 5.2 | 12 |
| Device 3-21 of the Invention | 14 | 5.2 | 10 |
| Device 3-22 of the Invention | 17 | 3.7 | 132 |
| Device 3-23 of the Invention | 17 | 3.8 | 127 |
| Device 3-24 of the Invention | 17 | 3.7 | 125 |
| Device 3-25 of the Invention | 17 | 3.9 | 121 |
| Device 3-26 of the Invention | 16 | 3.8 | 119 |
| Device 3-27 of the Invention | 17 | 3.9 | 124 |
| Device 3-28 of the Invention | 17 | 3.9 | 117 |
| Device 3-29 of the Invention | 16 | 3.9 | 109 |
| Device 3-30 of the Invention | 16 | 3.9 | 112 |
| Device 3-31 of the Invention | 16 | 4.1 | 117 |
| Device 3-1 for Comparison | 16 | 4.1 | 52 |
| Device 3-32 of the Invention | 16 | 4.0 | 121 |
| Device 3-2 for Comparison | 16 | 4.0 | 55 |
| Device 3-35 of the Invention | 16 | 4.1 | 80 |
| Device 3-36 of the Invention | 18 | 3.6 | 131 |
| Device 3-37 of the Invention | 18 | 4.3 | 143 |
| Device 3-3 for Comparison | 18 | 4.3 | 25 |
| Device 3-38 of the Invention | 18 | 4.5 | 135 |
| Device 3-4 for Comparison | 16 | 4.7 | 14 |
| Device 3-39 of the Invention | 16 | 4.2 | 78 |
| Device 3-40 of the Invention | 18 | 3.6 | 125 |
| Device 3-41 of the Invention | 18 | 3.7 | 122 |
| Device 3-42 of the Invention | 18 | 3.8 | 128 |

As shown in devices 3-1 to 3-42 of the examples in Tables 6 and 8, it has been found that devices of high performance can be obtained by the combination with various materials described in the specification.

Each device was manufactured in the same manner as in Example 2 except for carrying out deposition of the organic compound layers in the order of the following first layer to fifth layer.

First Layer: 2-TNATA and F₄-TCNQ (mass ratio: 99.7/0.3); film thickness: 60 nm
Second Layer: α-NPD; film thickness: 20 nm
Third Layer: H-10 and BD-1 (mass ratio: 97/3); film thickness: 40 nm
Fourth Layer Electron-transporting material shown in Table 9; film thickness: 10 nm
Fifth Layer: BAlq; film thickness: 10 nm

As a result of light emission of these devices, light emission resulting from the used light-emitting material was obtained from each device. Luminescent color is shown in Table 9. External quantum efficiency and driving durability of each device were measured in the same manner as in Example 2. The results obtained are shown in Table 9.

Incidentally, in Table 9, the synthesis method of the used electron-transporting material of the invention and sublimation purification fraction are inscribed as exemplified compound 1 (#A-B) (which means synthesis method A, sublimation purification fraction B).

TABLE 9

| | | | External | |
|---|---|---|---|---|
| | | | Quantum | |
| | Material of | Luminescent | Efficiency | Driving |
| Device No. | Fourth Layer | Color | (%) | Durability |
| Device 3-43 of | Exemplified | Blue | 4 | 1.0 |
| the Invention | Compound 1 | | | |
| | (#A-A) | | | |
| Device 3-5 for | Exemplified | Blue | 4 | 0.5 |
| Comparison | Compound 1 | | | |
| | (#B-A) | | | |
| Device 3-44 of | Exemplified | Blue | 4 | 0.9 |
| the Invention | Compound 5 | | | |
| | (#A-B) | | | |
| Device 3-6 for | Exemplified | Blue | 4 | 0.3 |
| Comparison | Compound 5 | | | |
| | (#B-A) | | | |
| Device 3-45 of | Exemplified | Blue | 4 | 0.8 |
| the Invention | Compound 36 | | | |
| | (#B-A) | | | |
| Device 3-7 for | Exemplified | Blue | 4 | <0.1 |
| Comparison | Compound 36 | | | |
| | (#B-B) | | | |
| Device 3-46 of | Exemplified | Blue | 4 | 0.9 |
| the Invention | Compound 40 | | | |
| | (#A-B) | | | |
| Device 3-8 for | Exemplified | Blue | 3 | <0.1 |
| Comparison | Compound 40 | | | |
| | (#A-No | | | |
| | sublimation) | | | |
| Device 3-47 of | Exemplified | Blue | 4 | 0.9 |
| the Invention | Compound 42 | | | |
| | (#A-B) | | | |
| Device 3-9 for | Exemplified | Blue | 3 | <0.1 |
| Comparison | Compound 42 | | | |
| | (#A-No | | | |
| | sublimation) | | | |
| Device 3-48 of | Exemplified | Blue | 4 | 0.7 |
| the Invention | Compound 46 | | | |
| | (#B-A) | | | |
| Device 3-10 for | Exemplified | Blue | 4 | 0.2 |
| Comparison | Compound 46 | | | |
| | (#B-B) | | | |
| Device 3-49 of | Exemplified | Blue | 4 | 0.7 |
| the Invention | Compound 51 | | | |
| | (#A-A) | | | |
| Device 3-11 for | Exemplified | Blue | 4 | <0.1 |
| Comparison | Compound 51 | | | |
| | (#A-No | | | |
| | sublimation) | | | |
| Device 3-50 of | Exemplified | Blue | 4 | 0.7 |
| the Invention | Compound 52 | | | |
| | (#A-B) | | | |
| Device 3-12 for | Exemplified | Blue | 4 | 0.3 |
| Comparison | Compound 52 | | | |
| | (#A-C) | | | |

As described above, it has been found that high performance devices can be similarly obtained by using the electron-transporting materials of the invention even when the structures of light-emitting materials and host materials and electron transporting materials to be combined are entirely different.

### Example 4

Concerning exemplified compound 1 of the charge-transporting material synthesized in Example 1 by synthesis method A, electron-transporting material samples were manufactured by varying the number of times of sublimation purification from 1 time to 7 times. Devices 4-1 to 4-6 of the invention were formed with the manufactured electron-transporting material samples in the same manner as in the manufacture of device 1-1 of the invention in Example 2, and external quantum efficiency and driving durability of these devices were evaluated. The results of evaluations are shown in Table 10 below.

TABLE 10

| | | | Number of Times | Content of | External Quantum | Driving |
|---|---|---|---|---|---|---|
| | Electron Transporting | Synthesis | of Sublimation | Impurity 1 | Efficiency | Dura- |
| Device No. | Material | Method | Purification | (% by mass) | (%) | bility |
| Device 1-1 of the Invention | Exemplified Compound 1 | A | 1 | 0.09 | 12 | 1.0 |
| Device 4-1 of the Invention | Exemplified Compound 1 | A | 2 | 0.08 | 12 | 1.1 |
| Device 4-2 of the Invention | Exemplified Compound 1 | A | 3 | 0.05 | 12 | 1.2 |
| Device 4-3 of the Invention | Exemplified Compound 1 | A | 4 | 0.03 | 12 | 1.3 |
| Device 4-4 of the Invention | Exemplified Compound 1 | A | 5 | 0.02 | 12 | 1.3 |
| Device 4-5 of the Invention | Exemplified Compound 1 | A | 6 | 0.01 | 12 | 1.3 |
| Device 4-6 of the Invention | Exemplified Compound 1 | A | 7 | <0.01 | 12 | 1.3 |

It has been found from the results shown in Table 10 that efficiency and durability hardly change when the content of impurity 1 is about 0.03% by mass or less, and environmental load increases with the increase of the number of process due to the increase in the number of times of sublimation purification.

Concerning exemplified compound 1 of the charge-transporting material synthesized in Example 1 by synthesis method A, electron-transporting material samples each different in the content of impurity 1 were manufactured by recrystallization and silica gel column chromatography without performing sublimation purification. Device 4-7 of the invention and devices 4-1 to 4-7 for comparison were formed with the manufactured charge-transporting material samples in the same manner as in the manufacture of device 1-1 of the invention in Example 2, and external quantum efficiency and driving durability of these devices were evaluated. The results of evaluations are shown in Table 11 below.

TABLE 11

| | | | | Content of | External Quantum | Driving |
|---|---|---|---|---|---|---|
| | Electron Transporting | Synthesis | | Impurity 1 | Efficiency | Dura- |
| Device No. | Material | Method | Purification Method | (% by mass) | (%) | bility |
| Device 4-1 for Comparison | Exemplified Compound 1 | A | Recrystallization | 2.8 | 9 | 0.05 |
| Device 4-2 for Comparison | Exemplified Compound 1 | A | Recrystallization (two times) | 1.0 | 9 | 0.1 |
| Device 4-3 for Comparison | Exemplified Compound 1 | A | Silica gel column chromatography | 3.6 | 9 | 0.01 |
| Device 4-3 for Comparison | Exemplified Compound 1 | A | Silica gel column chromatography | 0.45 | 9 | 0.2 |
| | | | (two times) | | | |
| Device 4-3 for Comparison | Exemplified Compound 1 | A | Silica gel column chromatography | 0.2 | 11 | 0.4 |
| | | | and recrystallization | | | |
| Device 4-3 for Comparison | Exemplified Compound 1 | A | Silica gel column chromatography | 0.13 | 10 | 0.55 |
| | | | and two times of recrystallization | | | |
| Device 4-3 for Comparison | Exemplified Compound 1 | A | Silica gel column chromatography | 0.11 | 12 | 0.7 |
| | | | and three times of recrystallization | | | |
| Device 4-7 of the Invention | Exemplified Compound 1 | A | Silica gel column chromatography | 0.1 | 12 | 0.8 |
| | | | and four times of recrystallization | | | |

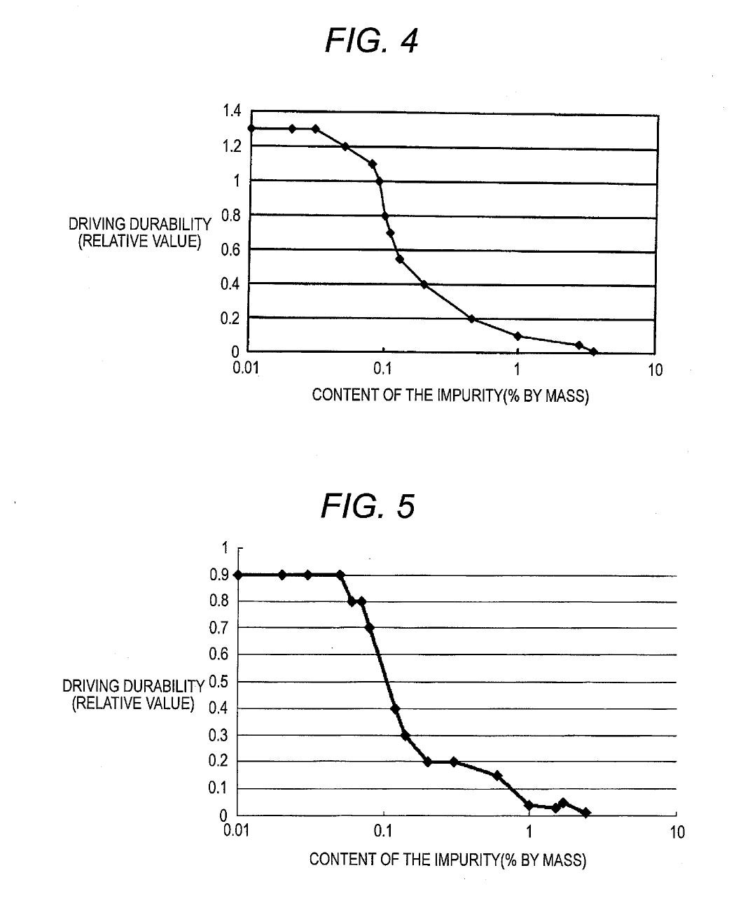

FIG. 4 is a graph showing variation of driving durability of devices to the content of impurity 1 based on the results shown in Tables 10 and 11.

It can be understood from FIG. 4 that the durability of the devices is conspicuously improved when the content of impurity 1 is 0.1% by mass or less.

### Example 5

Similarly to Example 4, charge-transporting material samples each different in the content of impurity 1 were manufactured with exemplified compound 6 by varying the number of times of sublimation purification and method of purification in the samples not subjected to purification sublimation. Devices 5-1 to 5-6 of the invention and devices 5-1 to 5-9 for comparison were formed with the manufactured electron-transporting material samples in the same manner as in the manufacture of device 1-1 of the invention in Example 2, and external quantum efficiency and driving durability of these devices were evaluated. The results of evaluations are shown in Table 12 below with the results of device 1-6 of the invention manufactured in Example 2.

TABLE 12

| | | | | Content of | External Quantum | Driving |
|---|---|---|---|---|---|---|
| | Electron Transporting | Synthesis | | Impurity 1 | Efficiency | Dura- |
| Device No. | Material | Mehod | Purification Method | (% by mass) | (%) | bility |
| Device 1-6 of | Exemplified Compound 6 | A | Sublimation purification (one time) | 0.08 | 13 | 0.7 |
| the Invention | | | | | | |
| Device 5-1 of | Exemplified Compound 6 | A | Sublimation purification (two times) | 0.06 | 13 | 0.8 |
| the Invention | | | | | | |
| Device 5-2 of | Exemplified Compound 6 | A | Sublimation purification (three times) | 0.05 | 13 | 0.9 |
| the Invention | | | | | | |
| Device 5-3 of | Exemplified Compound 6 | A | Sublimation purification (four times) | 0.03 | 13 | 0.9 |
| the Invention | | | | | | |
| Device 5-4 of | Exemplified Compound 6 | A | Sublimation purification (five times) | 0.02 | 13 | 0.9 |
| the Invention | | | | | | |
| Device 5-5 of | Exemplified Compound 6 | A | Sublimation purification (six times) | 0.01 | 13 | 0.9 |
| the Invention | | | | | | |
| Device 5-1 for | Exemplified Compound 6 | A | Recrystallization | 2.4 | 9 | 0.01 |
| Comparison | | | | | | |
| Device 5-2 for | Exemplified Compound 6 | A | Recrystallization (two times) | 1.5 | 10 | 0.03 |
| Comparison | | | | | | |
| Device 5-3 for | Exemplified Compound 6 | A | Silica gel column chromatography | 1.7 | 9 | 0.05 |
| Comparison | | | | | | |
| Device 5-4 for | Exemplified Compound 6 | A | Silica gel column chromatography (two times) | 0.6 | 12 | 0.15 |
| Comparison | | | | | | |
| Device 5-5 for | Exemplified Compound 6 | A | Silica gel column chromatography (three times) | 0.2 | 12 | 0.2 |
| Comparison | | | | | | |
| Device 5-6 for | Exemplified Compound 6 | A | Silica gel column chromatography and | 1.0 | 11 | 0.04 |
| Comparison | | | recrystallization | | | |
| Device 5-7 for | Exemplified Compound 6 | A | Silica gel column chromatography and | 0.3 | 12 | 0.2 |
| Comparison | | | two times of recrystallization | | | |
| Device 5-8 for | Exemplified Compound 6 | A | Two times of silica gel column chromatography | 0.14 | 13 | 0.3 |
| Comparison | | | and recrystallization | | | |
| Device 5-6 of | Exemplified Compound 6 | A | Two times of silica gel column chromatography | 0.07 | 13 | 0.8 |
| the Invention | | | and two times of recrystallization | | | |
| Device 5-9 for | Exemplified Compound 6 | A | Three times of silica gel column chromatography | 0.12 | 12 | 0.4 |
| Comparison | | | and recrystallization | | | |

It has been found from the results shown in Table 12 that efficiency and durability hardly change when the content of impurity 1 is about 0.05% by mass or less, and environmental load increases with the increase of the number of process due to the increase in the number of times of sublimation purification.

FIG. 5 is a graph showing variation of driving durability of the devices to the content of impurity 1.

It can be understood from FIG. 5 that the durability of the devices is conspicuously improved when the content of impurity 1 is 0.1% by mass or less.

### Example 6

Similarly to Example 4, charge-transporting material samples each different in the content of impurity 1 were manufactured with exemplified compound 51 by varying the number of times of sublimation purification and method of purification in the samples not subjected to purification sublimation. Devices 6-1 to 6-4 of the invention and devices 6-1 to 6-5 for comparison were formed with the manufactured electron-transporting material samples in the same manner as in the manufacture of device 1-1 of the invention in Example 2, and external quantum efficiency and driving durability of these devices were evaluated. The results of evaluations are shown in Table 13 below with the results of device 1-27 of the invention manufactured in Example 2.

TABLE 13

| | | | | Content of | External Quantum | Driving |
|---|---|---|---|---|---|---|
| | Electron Transporting | Synthesis | | Impurity 1 | Efficiency | Dura- |
| Device No. | Material | Method | Purification Method | (% by mass) | (%) | bility |
| Device 1-27 of | Exemplified Compound 51 | A | Sublimation purification (one time) | 0.09 | 13 | 1.1 |
| the Invention | | | | | | |
| Device 6-1 of | Exemplified Compound 51 | A | Sublimation purification (two times) | 0.04 | 13 | 1.1 |
| the Invention | | | | | | |
| Device 6-2 of | Exemplified Compound 51 | A | Sublimation purification (three times) | 0.01 | 13 | 1.1 |
| the Invention | | | | | | |
| Device 6-3 of | Exemplified Compound 51 | A | Sublimation purification (four times) | 0.01 | 13 | 1.1 |
| the Invention | | | | | | |
| Device 6-1 for | Exemplified Compound 51 | A | Silica gel column chromatography | 2.5 | 12 | 0.01 |
| Comparison | | | | | | |
| Device 6-2 for | Exemplified Compound 51 | A | Silica gel column chromatography and | 1.3 | 12 | 0.02 |
| Comparison | | | recrystallization | | | |
| Device 6-3 for | Exemplified Compound 51 | A | Silica gel column chromatography | 0.50 | 13 | 0.1 |
| Comparison | | | and two times of recrystallization | | | |
| Device 6-4 for | Exemplified Compound 51 | A | Silica gel column chromatography | 0.17 | 13 | 0.5 |
| Comparison | | | and three times of recrystallization | | | |
| Device 6-5 for | Exemplified Compound 51 | A | Two times of silica gel column chromatography | 0.15 | 13 | 0.7 |
| Comparison | | | and recrystallization | | | |
| Device 6-4 of | Exemplified Compound 51 | A | Two times of silica gel column chromatography | 0.08 | 13 | 1.1 |
| the Invention | | | and two times of recrystallization | | | |

It has been found from the results shown in Table 13 that efficiency and durability hardly change when the content of impurity 1 is about 0.09% by mass or less, and environmental load increases with the increase of the number of process due to the increase in the number of times of sublimation purification.

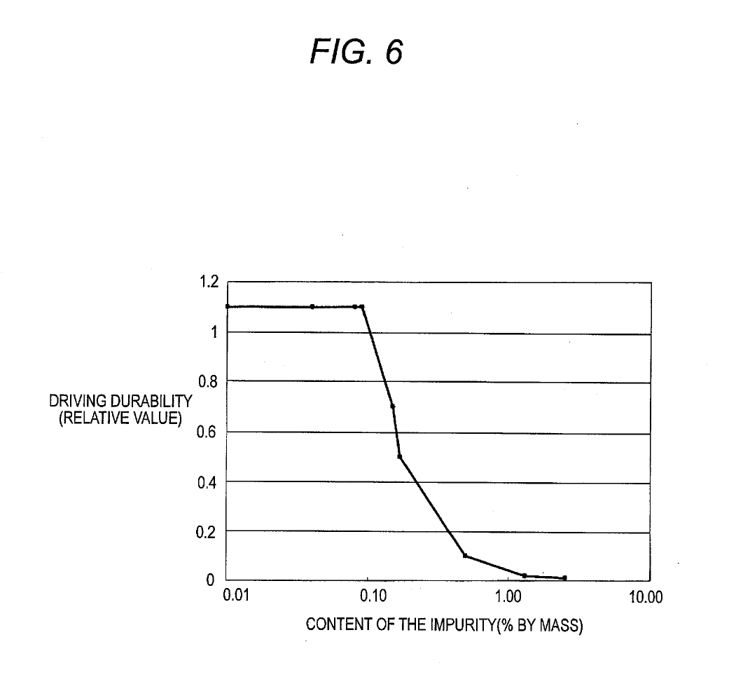

FIG. 6 is a graph showing variation of driving durability of the devices to the content of impurity 1.

It can be understood from FIG. 6 that the durability of the devices is conspicuously improved when the content of impurity 1 is 0.1% by mass or less.

### Example 7

Similarly to Example 4, charge-transporting material samples each different in the content of impurity 1 were manufactured with exemplified compound 52 by varying the number of times of sublimation purification and method of purification in the samples not subjected to purification sublimation. Devices 7-1 to 7-4 of the invention and devices 7-1 to 7-6 for comparison were formed with the manufactured electron-transporting material samples in the same manner as in the manufacture of device 1-1 of the invention in Example 2, and external quantum efficiency and driving durability of these devices were evaluated. The results of evaluations are shown in Table 14 below with the results of device 1-29 of the invention manufactured in Example 2.

TABLE 14

| | | | | Content of | External Quantum | Driving |
|---|---|---|---|---|---|---|
| | Electron Transporting | Synthesis | | Impurity 1 | Efficiency | Dura- |
| Device No. | Material | Method | Purification Method | (% by mass) | (%) | bility |
| Device 1-29 of | Exemplified Compound 52 | A | Sublimation purification (one time) | 0.05 | 9 | 0.8 |
| the Invention | | | | | | |
| Device 7-1 of | Exemplified Compound 52 | A | Sublimation purification (two times) | 0.03 | 9 | 0.8 |
| the Invention | | | | | | |
| Device 7-2 of | Exemplified Compound 52 | A | Sublimation purification (three times) | 0.01 | 9 | 0.8 |
| the Invention | | | | | | |
| Device 7-3 of | Exemplified Compound 52 | A | Sublimation purification (four times) | 0.01 | 9 | 0.8 |
| the Invention | | | | | | |
| Device 7-1 for | Exemplified Compound 52 | A | Recrystallization | 2.7 | 7 | 0.01 |
| Comparison | | | | | | |
| Device 7-2 for | Exemplified Compound 52 | A | Recrystallization (two times) | 1.3 | 7 | 0.01 |
| Comparison | | | | | | |
| Device 7-3 for | Exemplified Compound 52 | A | Silica gel column chromatography | 0.9 | 8 | 0.01 |
| Comparison | | | | | | |
| Device 7-4 for | Exemplified Compound 52 | A | Silica gel column chromatography and | 0.5 | 8 | 0.03 |
| Comparison | | | recrystallization | | | |
| Device 7-5 for | Exemplified Compound 52 | A | Silica gel column chromatography and | 0.2 | 9 | 0.1 |
| Comparison | | | two times of recrystallization | | | |
| Device 7-6 for | Exemplified Compound 52 | A | Silica gel column chromatography and | 0.12 | 9 | 0.15 |
| Comparison | | | three times of recrystallization | | | |
| Device 7-4 of | Exemplified Compound 52 | A | Silica gel column chromatography and | 0.07 | 9 | 0.75 |
| the Invention | | | four times of recrystallization | | | |

It has been found from the results shown in Table 14 that efficiency and durability hardly change when the content of impurity 1 is about 0.05% by mass or less, and environmental load increases with the increase of the number of process due to the increase in the number of times of sublimation purification.

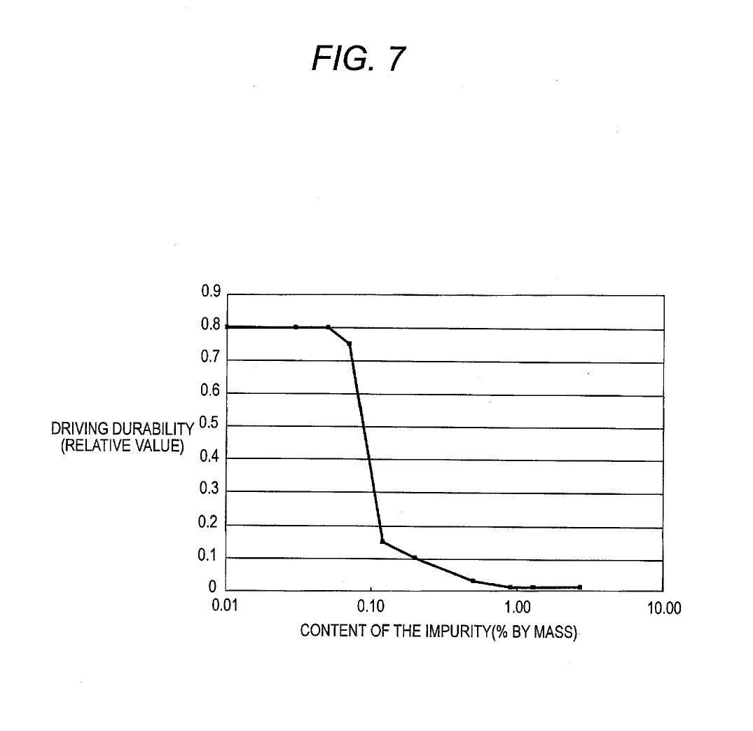

FIG. 7 is a graph showing variation of driving durability of the devices to the content of impurity 1.

It can be understood from FIG. 7 that the durability of the devices is conspicuously improved when the content of impurity 1 is 0.1% by mass or less.

### Example 8

Similarly to Example 7, charge-transporting material samples each different in the content of impurity 2 were manufactured with exemplified compound 54 by varying the number of times of sublimation purification and method of purification in the samples not subjected to purification sublimation. Devices 8-1 to 8-6 of the invention and devices 8-1 to 8-5 for comparison were formed with the manufactured electron-transporting material samples in the same manner as in the manufacture of device 1-1 of the invention in Example 2, and external quantum efficiency and driving durability of these devices were evaluated. The results of evaluations are shown in Table 15 below with the results of device 1-33 of the invention manufactured in Example 2.

TABLE 15

| | | | | Content of | External Quantum | Driving |
|---|---|---|---|---|---|---|
| | Electron Transporting | Synthesis | | Impurity 2 | Efficiency | Dura- |
| Device No. | Material | Method | Purification Method | (% by mass) | (%) | bility |
| Device 1-33 of | Exemplified Compound 54 | B | Sublimation purification (one time) | 0.04 | 9 | 1.1 |
| the Invention | | | | | | |
| Device 8-1 of | Exemplified Compound 54 | B | Sublimation purification (two times) | 0.02 | 9 | 1.1 |
| the Invention | | | | | | |
| Device 8-2 of | Exemplified Compound 54 | B | Sublimation purification (three times) | 0.01 | 9 | 1.1 |
| the Invention | | | | | | |
| Device 8-3 of | Exemplified Compound 54 | B | Sublimation purification (four times) | 0.01 | 9 | 1.1 |
| the Invention | | | | | | |
| Device 8-4 of | Exemplified Compound 54 | B | Sublimation purification (five times) | 0.01 | 9 | 1.1 |
| the Invention | | | | | | |
| Device 8-1 for | Exemplified Compound 54 | B | Recrystallization | 2.9 | 7 | 0.05 |
| Comparison | | | | | | |
| Device 8-2 for | Exemplified Compound 54 | B | Recrystallization (two times) | 1.8 | 8 | 0.05 |
| Comparison | | | | | | |
| Device 8-3 for | Exemplified Compound 54 | B | Silica gel column chromatography | 1.1 | 8 | 0.05 |
| Comparison | | | | | | |
| Device 8-4 for | Exemplified Compound 54 | B | Silica gel column chromatography and | 0.5 | 8 | 0.07 |
| Comparison | | | recrystallization | | | |
| Device 8-5 for | Exemplified Compound 54 | B | Silica gel column chromatography | 0.15 | 9 | 0.3 |
| Comparison | | | and two times of recrystallization | | | |
| Device 8-6 of | Exemplified Compound 54 | B | Silica gel column chromatography | 0.08 | 9 | 1 |
| the Invention | | | and three times of recrystallization | | | |

It has been found from the results shown in Table 15 that efficiency and durability hardly change when the content of impurity 2 is about 0.04% by mass or less, and environmental load increases with the increase of the number of process due to the increase in the number of times of sublimation purification.

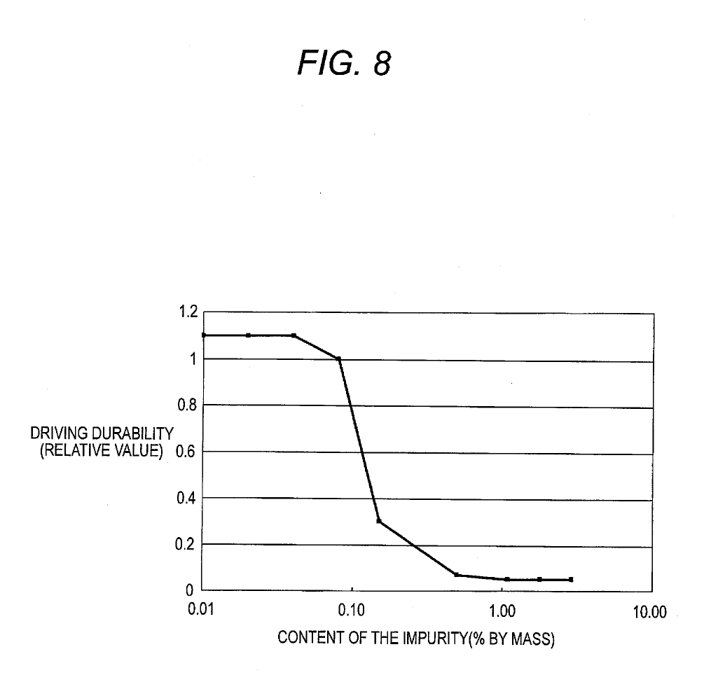

FIG. 8 is a graph showing variation of driving durability of the devices to the content of impurity 2.

It can be understood from FIG. 8 that the durability of the device is conspicuously improved when the content of impurity 2 is 0.1% by mass or less.

Further, in the case of light emission apparatus, display apparatus and illumination apparatus, it is required to instantaneously emit light in high luminance through high current density at each pixel part, and the luminescence devices of the invention can be advantageously used in such a case, since the devices are designed to be capable of obtaining high light emission efficiency.

The structures of the compounds used in Examples 2 to 8 are shown below.

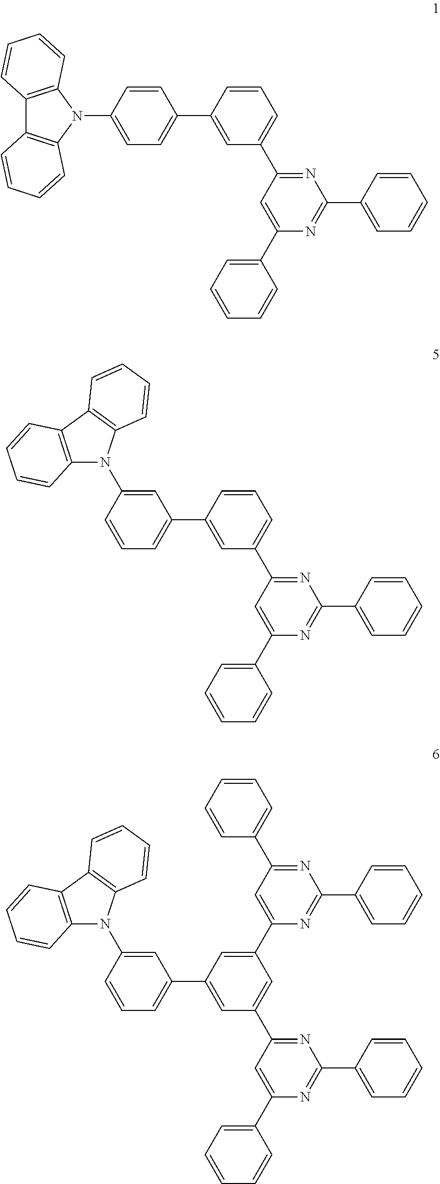

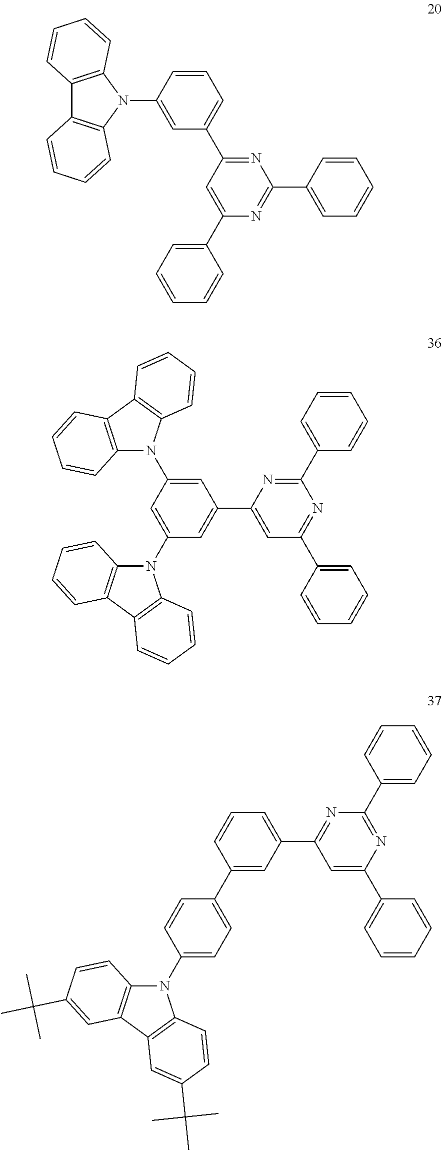

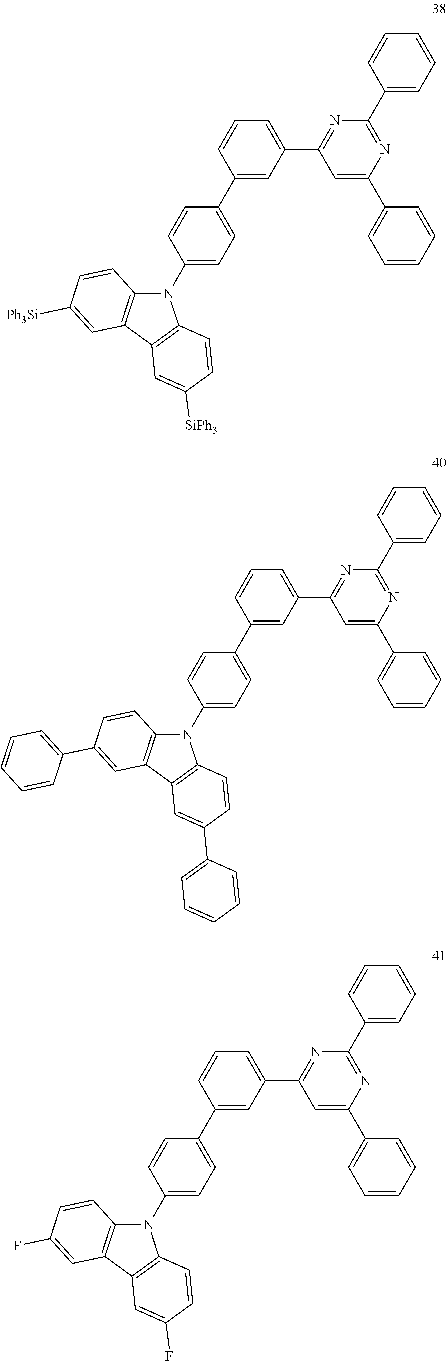

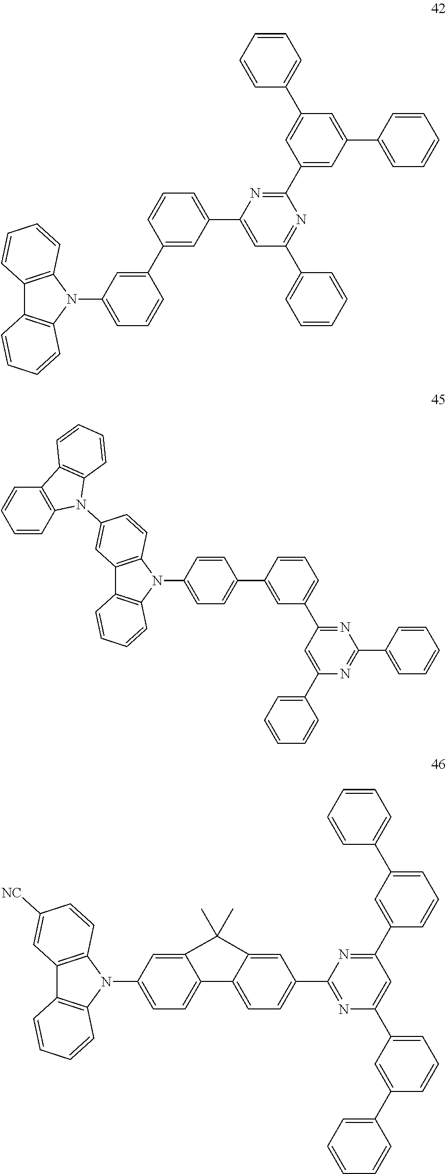

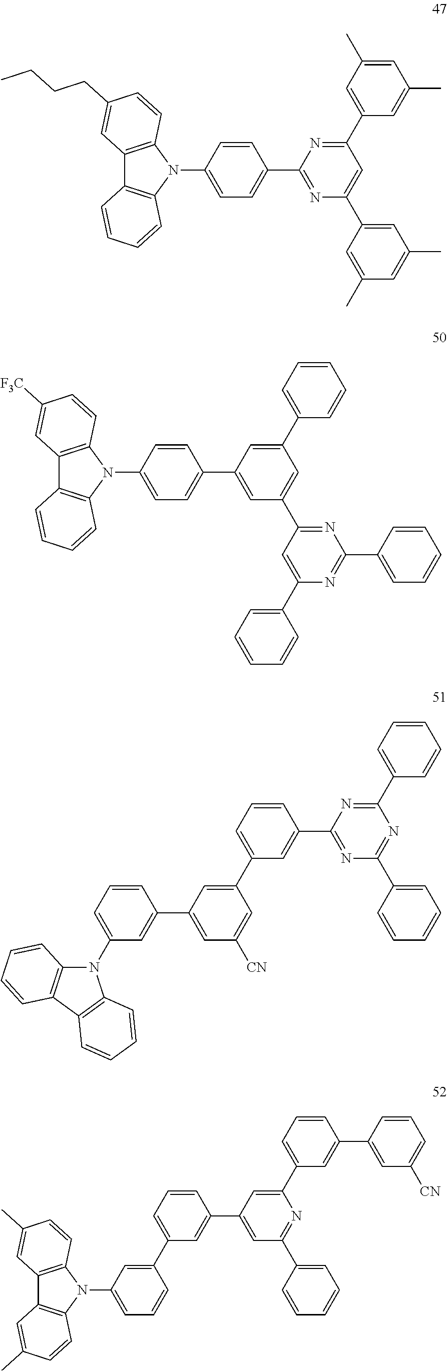

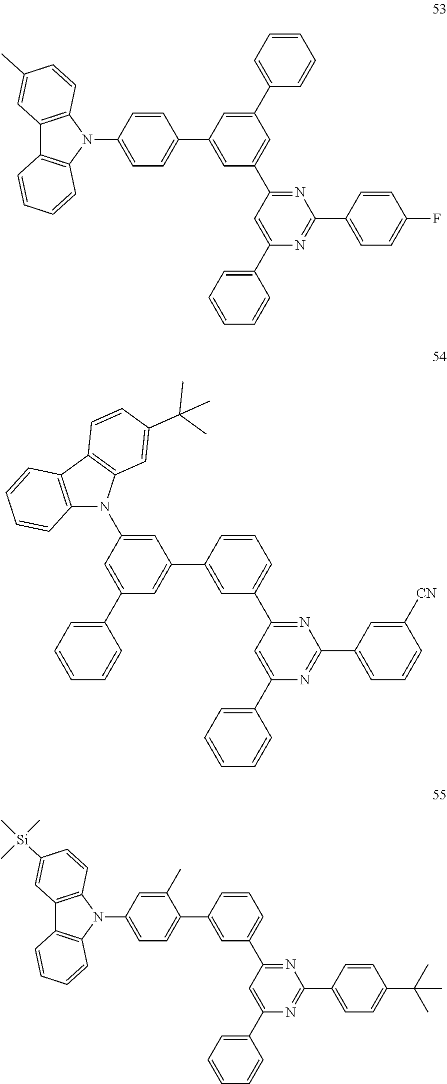

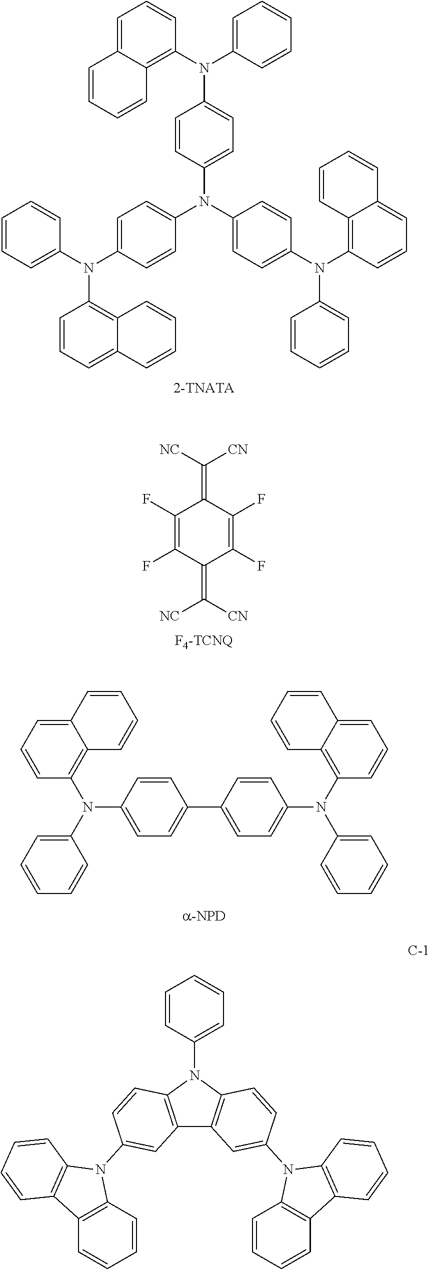

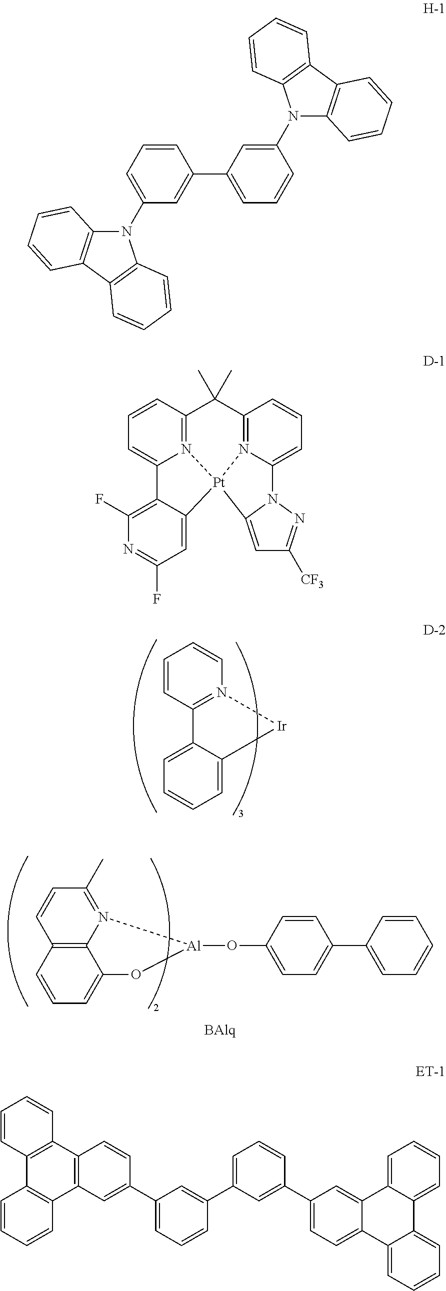

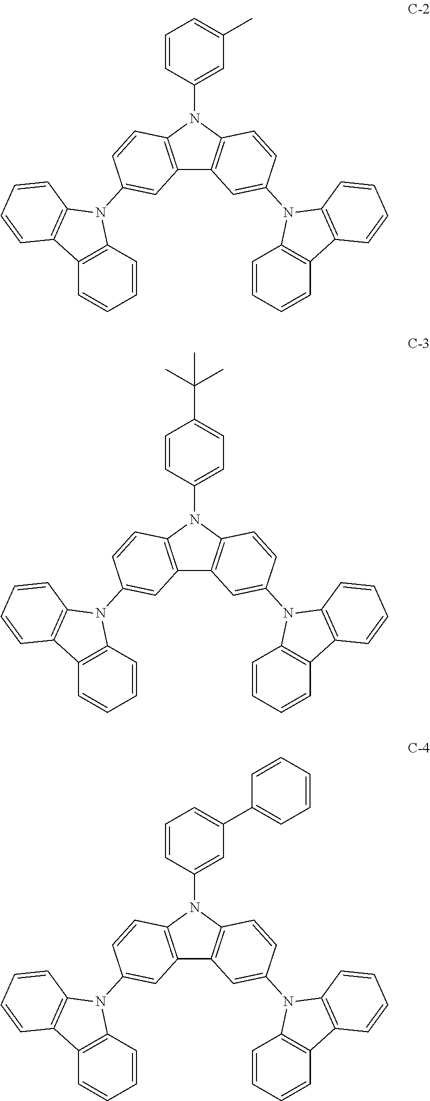

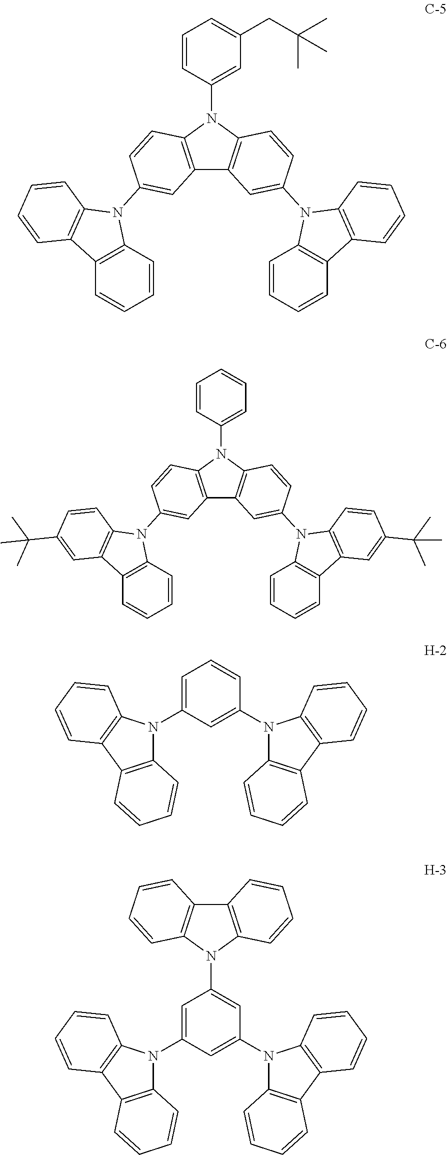

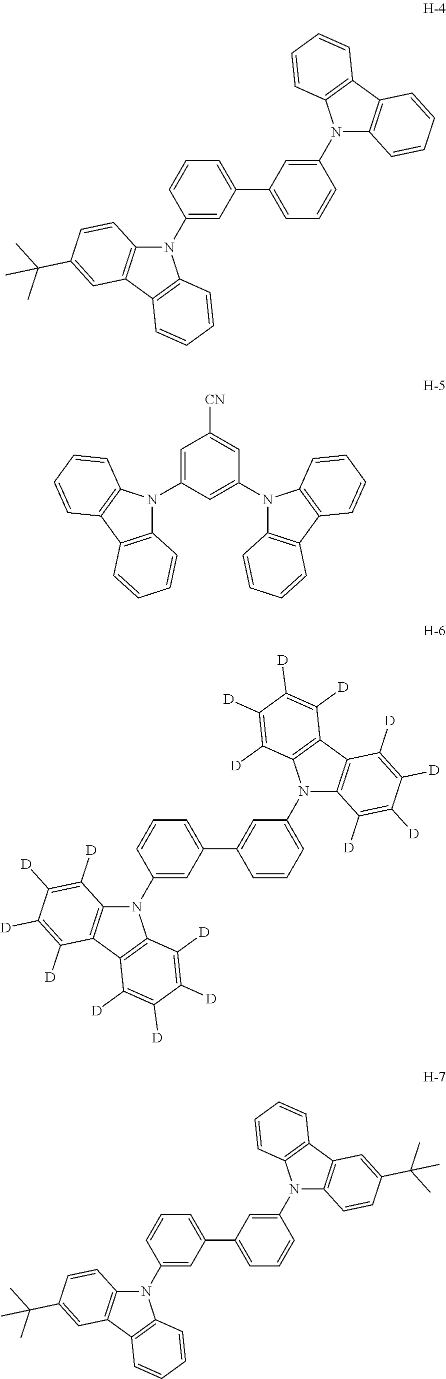

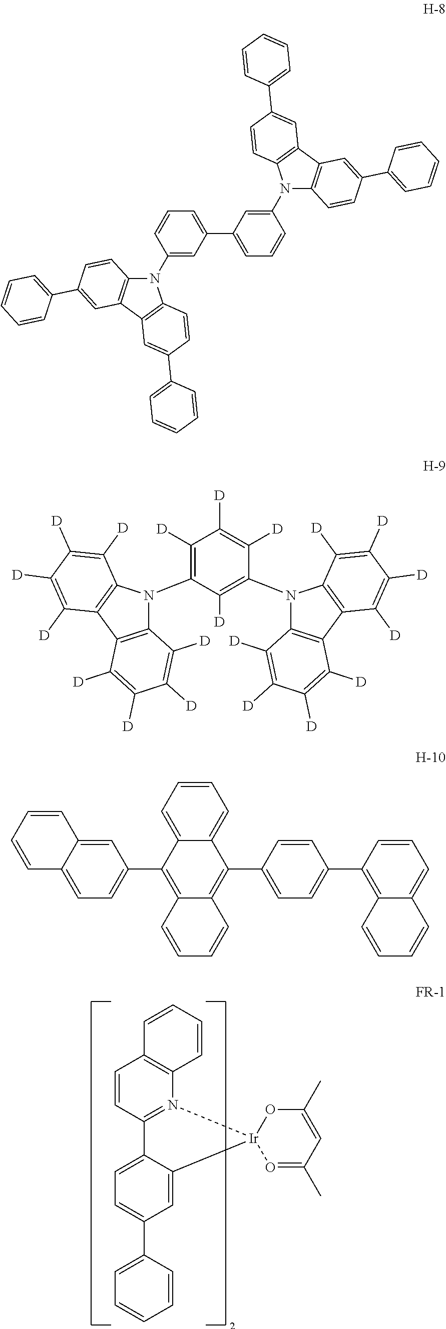

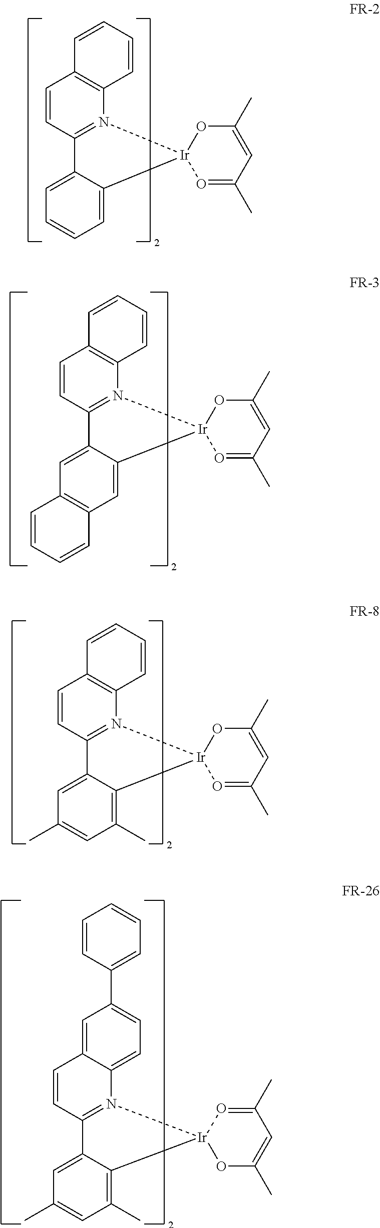

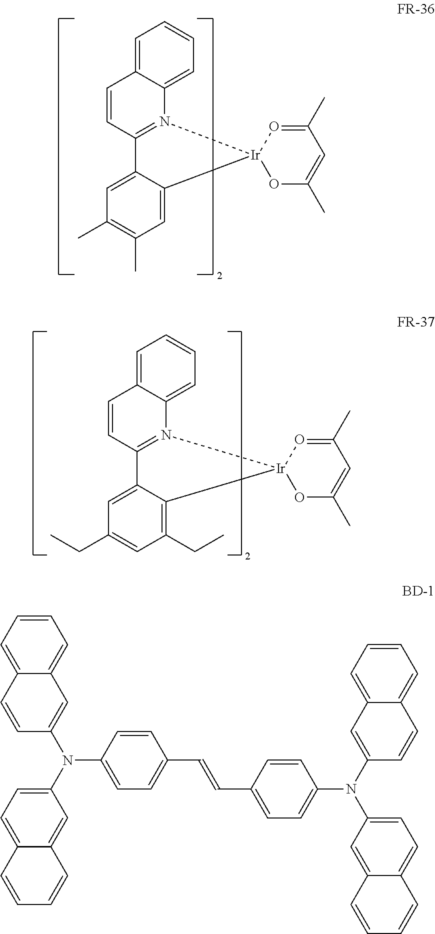

### INDUSTRIAL APPLICABILITY

The organic electroluminescence devices using the charge-transporting material of the invention can be preferably used in display devices, displays, backlights, electrophotography, light sources for illumination, light sources for recording, light sources for exposure, light sources for reading, indicators, signboards, interior designs, optical communications, and the like. The organic electroluminescence devices in the invention can be particularly preferably used in devices driven in the region high in light emission luminance such as illumination apparatus and display apparatus.

While the invention has been described in detail with reference to specific examples thereof, it will be apparent to one skilled in the art that various changes and modifications can be made therein without departing from the spirit and scope thereof.

This patent application is based on Japanese patent application filed on Jul. 31, 2009 (Japanese Patent Application No. 2009-180226), Japanese patent application filed on Aug. 31, 2009 (Japanese Patent Application No. 2009-201158), and Japanese patent application filed on May 7, 2010 (Japanese Patent Application No. 2010-107586), and the contents of which are incorporated herein by reference.

### DESCRIPTION OF REFERENCE NUMERALS

2: Substrate
3: Anode
4: Hole-injecting layer
5: Hole-transporting layer
6: Light-emitting layer
7: Hole-blocking layer
8: Electron-transporting layer
9: Cathode
10: Organic electroluminescence device
11: Organic layer

1. A charge-transporting material comprising a compound represented by the following formula (1), wherein contents of a compound represented by the following formula (I-1) and a compound represented by the following formula (I-2) are respectively 0.1% by mass or less to the compound represented by formula (1): 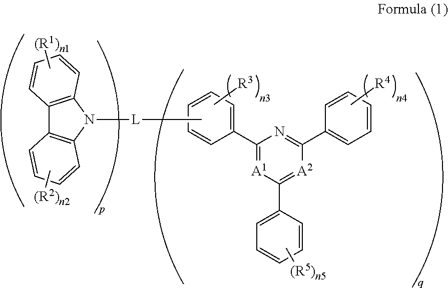
wherein each of A¹ and A² independently represents N, —CH or —CR; R represents a substituent; L represents a single bond, an arylene group, a cycloalkylene group or an aromatic heterocyclic group, a ring may be formed by the carbon atom in the benzene ring to which L is bonded, the atom in L, and further another atom, the other atom is a carbon atom, an oxygen atom or a sulfur atom, and the carbon atom may further have a substituent; each of R¹ to R⁵ independently represents a substituent; each of n1, n2 and n3 independently represents an integer of 0 to 4; each of n4 and n5 independently represents an integer of 0 to 5; and each of p and q independently represents an integer of 1 to 4; 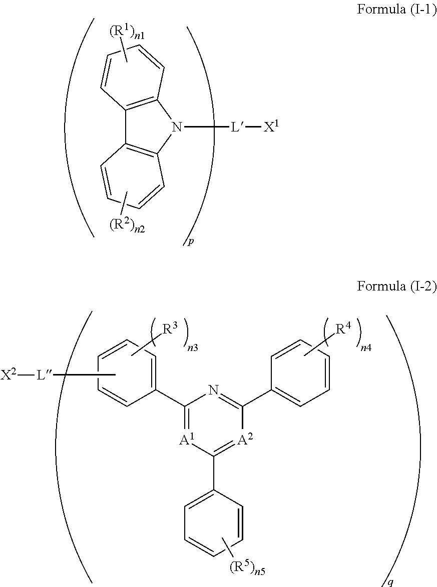
wherein in formulae (I-1) and (1-2) each of A′, A², R¹ to R⁵, n1 to n5, p and q has the same meaning as in formula (1), and each is the same group or integer with that represented by each of A¹, A², R¹ to R⁵, n1 to n5, p and q in formula (1); each of X′ and X² independently represents a halogen atom; and each of L′ and L″ has the same meaning with L.

2. The charge-transporting material as claimed in claim 1, wherein the contents of the compound represented by formula (I-1) and the compound represented by formula (I-2) are respectively 0.001% by mass or more and 0.1% by mass or less to the compound represented by formula (1).

3. The charge-transporting material as claimed in claim 1, wherein in formula (1), either of A¹ and A² represents a nitrogen atom and the other represents —CH or —CR; and R represents a substituent.

4. The charge-transporting material as claimed in claim 1, wherein in formula (1), L represents a single bond, a phenylene group, a biphenylene group, or a terphenylene group.

5. The charge-transporting material as claimed in claim 1, wherein in formula (1), each of R¹ to R⁵ independently represents a halogen atom, an alkyl group, an aryl group, an aromatic heterocyclic group, an adamantyl group, a cyano group, a silyl group, or a carbazolyl group.

6. The charge-transporting material as claimed in claim 1, wherein in formula (1), each of R¹ to R⁵ independently represents an alkyl group, an aryl group, a cyano group, or a silyl group.

7. The charge-transporting material as claimed in claim 1, wherein in formula (1), all of n1 to n5 represent 0.

8. The charge-transporting material as claimed in claim 1, wherein the compound represented by formula (1) is a compound represented by the following formula (2): 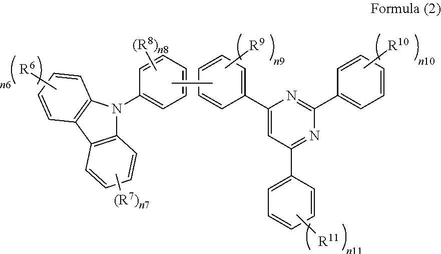
wherein each of R⁶ to R¹¹ independently represents an alkyl group, an aryl group, a cyano group, or a silyl group; each of n6 to n9 independently represents an integer of 0 to 4; and each of n10 and n11 independently represents an integer of 0 to 5.

9. The charge-transporting material as claimed in claim 1, wherein in formula (2), all of n6 to n11 represent 0.

10. The charge-transporting material as claimed in claim 8, wherein the compound represented by formula (I-1) and the compound represented by formula (I-2) are respectively a compound represented by the following formula (1′-1) and a compound represented by the following formula (II-2): 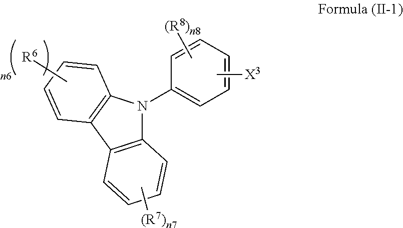 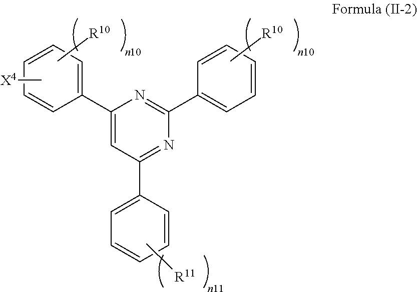
wherein in formulae (II-1) and (II-2), each of X³ and X⁴ independently represents a halogen atom; and each of R⁶ to R¹¹ and n6 to n11 has the same meaning as in formula (2).

11. The charge-transporting material as claimed in claim 1, wherein a molecular weight of the compound represented by formula (1) is 450 or more and 800 or less.

12. The charge-transporting material as claimed in claim 1, wherein a minimum triplet excited state T₁ energy of the compound represented by formula (1) in a film state is 2.69 eV or more and 3.47 eV or less.

13. The charge-transporting material as claimed in claim 1, wherein a glass transition temperature Tg of the compound represented by formula (1) is 80° C. or more and 400° C. or less.

14. A method for manufacturing the compound represented by the following formula (2), which comprises a step of performing a coupling reaction of a compound represented by the following formula (M1) with a compound represented by the following formula (M2) by using a palladium catalyst: 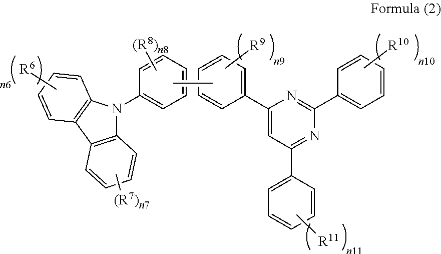
wherein each of R⁶ to R¹¹ independently represents an alkyl group, an aryl group, a cyano group, or a silyl group; each of n6 to n9 independently represents an integer of 0 to 4; and each of n10 and n11 independently represents an integer of 0 to 5; 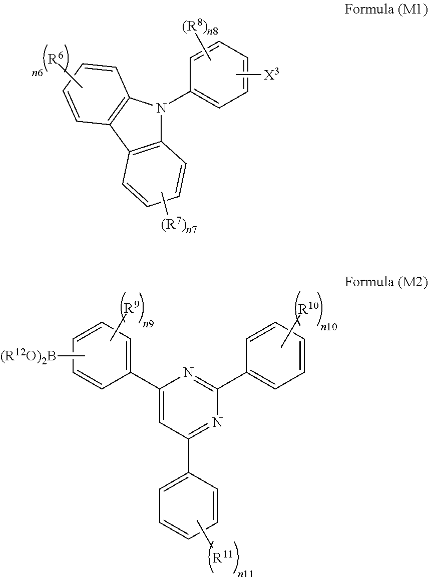
wherein in formulae (M1) and (M2), X³ represents a halogen atom; each of R⁶ to R¹¹ and n6 to n11 has the same meaning as in formula (2); and R¹² represents a hydrogen atom or an alkyl group.

15. The method as claimed in claim 14, which further comprises a step of performing a sublimation purification of a reaction product obtained by the coupling reaction.

16. The charge-transporting material as claimed in claim 8 or 9, wherein the compound represented by formula (2) is obtained by a method comprising a step of performing a coupling reaction of a compound represented by the following formula (M1) with a compound represented by the following formula (M2) by using a palladium catalyst: 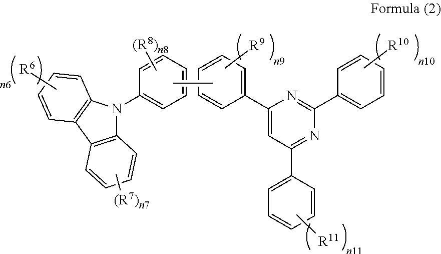
wherein each of R⁶ to R¹¹ independently represents an alkyl group, an aryl group, a cyano group, or a silyl group; each of n6 to n9 independently represents an integer of 0 to 4; and each of n10 and n11 independently represents an integer of 0 to 5; 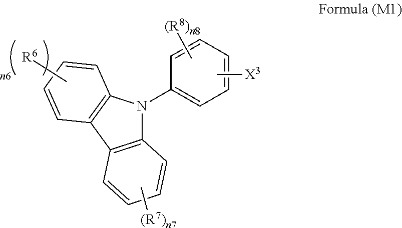 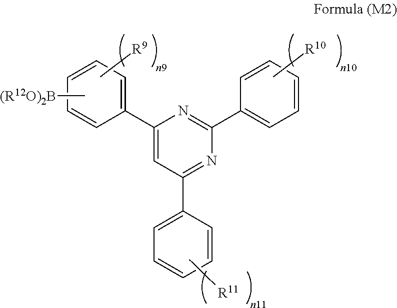
wherein in formulae (M1) and (M2), X³ represents a halogen atom; each of R⁶ to R¹¹ and n6 to n11 has the same meaning as in formula (2); and R¹² represents a hydrogen atom or an alkyl group.

17. An organic electroluminescence device comprising:
a pair of electrodes; and
at least one organic layer including a light-emitting layer between the pair of electrodes,
wherein a layer of the at least one organic layers contains the charge-transporting material as claimed in claim 1.

18. The organic electroluminescence device as claimed in claim 17, wherein the organic layer contains an electron-transporting layer, and the electron transporting layer contains the charge-transporting material.

19. The organic electroluminescence device as claimed in claim 17, wherein the light-emitting layer contains the charge-transporting material.

20. The organic electroluminescence device as claimed in claim 17, wherein the light-emitting layer contains a compound represented by the following formula (C-3) as a light-emitting material: 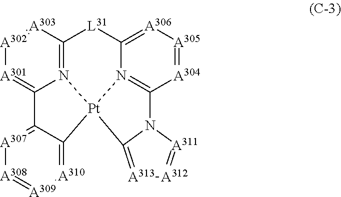
wherein each of A³⁰¹ to A³¹³ independently represents C—R or N; R represents a hydrogen atom or a substituent; and L³¹ represents a single bond or a divalent linking group.

21. The organic electroluminescence device as claimed in claim 20, wherein L³¹ represents a single bond, an alkylene group or an arylene group, the alkylene group or arylene group may further have an alkyl group or an aryl group as the substituent, and when two or more substituents are present, the two or more substituents may be bonded to form a ring.

22. The organic electroluminescence device as claimed in claim 20, wherein A³⁰² or A³⁰⁵ represents C—R; and R represents a hydrogen atom, an amino group, an alkoxy group, an aryloxy group or a fluorine group.

23. The organic electroluminescence device as claimed in claim 20, wherein A³⁰¹, A³⁰³, A³⁰⁴ or A³⁰⁶ represents C—R; and R represents a hydrogen atom, an amino group, an alkoxy group, an aryloxy group, or a fluorine group.

24. The organic electroluminescence device as claimed in claim 20, wherein when A³⁰⁷, A³⁰⁸, A³⁰⁹ or A³¹⁰ represents C—R, R represents a hydrogen atom, an alkyl group, a perfluoroalkyl group, an aryl group, a dialkylamino group, a cyano group, or a fluorine atom.

25. The organic electroluminescence device as claimed in claim 20, wherein the 6-membered ring formed by A³⁰⁷, A³⁰⁸, A³⁰⁹, A³¹⁰ and two carbon atoms is a benzene ring, a pyridine ring, a pyrazine ring, a pyrimidine ring, or a pyridazine ring.

26. The organic electroluminescence device as claimed in claim 20, wherein when A³¹¹, A³¹² or A³¹³ represents C—R, R represents a hydrogen atom, an alkyl group, a perfluoroalkyl group, an aryl group, a dialkylamino group, a cyano group, or a fluorine atom.

27. The organic electroluminescence device as claimed in claim 20, wherein at least one of A³¹¹, A³¹² and A³¹³ represents N.

28. The organic electroluminescence device as claimed in claim 17, wherein the light-emitting layer contains a compound represented by the following formula (PQ-1) as a light-emitting material: 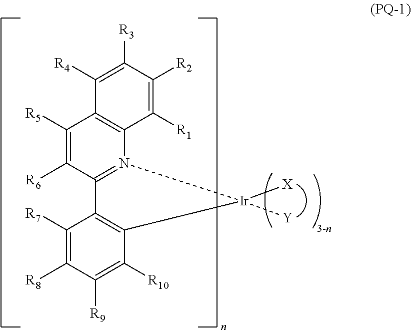
wherein each of R¹ to R¹⁰ independently represents a hydrogen atom or a substituent, and the substituents may be bonded to each other to form a ring; X—Y represents a bidentate monoanionic ligand; and n represents an integer of 1 to 3.

29. The organic electroluminescence device as claimed in claim 28, wherein each of R₁ to R₁₀ independently represents a hydrogen atom, a methyl group, an ethyl group, an isopropyl group, a t-butyl group, a neopentyl group, an isobutyl group, a phenyl group, a naphthyl group, a phenanthryl group, or a tolyl group.

30. The organic electroluminescence device as claimed in claim 28, wherein X—Y represents acetylacetonate or picolinate.

31. The organic electroluminescence device as claimed in claim 28, wherein the compound represented by formula (PQ-1) is a compound represented by the following formula (PQ-3): 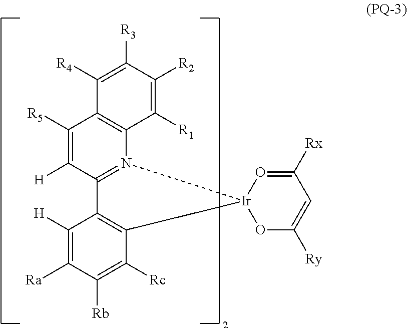
wherein each of R₁ to R₅ has the same meaning as in formula (PQ-1); each of Ra, Rb and Rc independently represents a hydrogen atom or an alkyl group, provided that one of Ra, Rb and Rc represents a hydrogen atom and each of other two represents an alkyl group; and each of Rx and Ry independently represents an alkyl group or a phenyl group.

32. A composition comprising the charge-transporting material as claimed in claim 1.

33. A light emission apparatus comprising the organic electroluminescence device as claimed in claim 17.

34. A display apparatus comprising the organic electroluminescence device as claimed in claim 17.

35. An illumination apparatus comprising the organic electroluminescence device as claimed in claim 17.
